(19) United States
(12) Patent Application Publication
(10) Pub. No.: US 2020/0087314 A1
(43) Pub. Date: Mar. 19, 2020

(54) HISTONE DEACETYLASE INHIBITORS

(71) Applicant: BIOMARIN PHARMACEUTICAL INC., Novato, CA (US)

(72) Inventors: Gregory Luedtke, Royal Oaks, CA (US); Shripad Bhagwat, Novato, CA (US)

(21) Appl. No.: 16/471,628

(22) PCT Filed: Dec. 22, 2017
(86) PCT No.: PCT/US17/68118

§ 371 Date: Jun. 20, 2019

Related U.S. Application Data
(60) Provisional application No. 62/437,767, filed on Dec. 22, 2016.

(51) Int. Cl. C07D 487/10; C07D 211/34; C07D 223/12; C07D 205/04; C07D 491/107; C07D 241/04; C07D 207/09

(52) U.S. Cl. CPC C07D 487/10; C07D 211/34; C07D 223/12; C07D 207/09; C07D 491/107; C07D 241/04; C07D 205/04

(57) ABSTRACT

Provided herein are compounds and methods for inhibiting histone deacetylase (“HDAC”) enzymes (e.g., HDAC1, HDAC2, and HDAC3).

### TECHNICAL FIELD

Provided herein are compounds and methods of inhibiting histone deacetylase (“HDAC”) enzymes (e.g., HDAC1, HDAC2, and HDAC3).

### BACKGROUND

To date, 18 HDAC enzymes have been identified in humans and there is increasing evidence that the 18 HDAC enzymes in humans are not redundant in function. HDAC enzymes are classified into three main groups based on their homology to yeast proteins. Class I includes HDAC1, HDAC2, HDAC3, and HDAC8 and have homology to yeast RPD3. HDAC4, HDAC5, HDAC7, and HDAC9 belong to class IIa and have homology to yeast HDAC1. HDAC6 and HDAC10 contain two catalytic sites and are classified as class IIb, whereas HDAC11 has conserved residues in its catalytic center that are shared by both class I and class II deacetylases and is placed in class IV. These HDAC enzymes contain zinc in their catalytic site and are inhibited by compounds like trichostatin A (TSA) and vorinostat [suberoylanilide hydroxamic acid (SAHA)]. Class III HDAC enzymes are known as sirtuins. They have homology to yeast Sir2, require NAD⁺ as cofactor, and do not contain zinc in the catalytic site. In general, HDAC inhibitors of zinc-dependent HDAC enzymes include a Zn-binding group, as well as a surface recognition domain.

HDAC enzymes are involved in the regulation of a number of cellular processes. Histone acetyltransferases (HATs) and HDAC enzymes acetylate and deacetylate lysine residues on the N termini of histone proteins thereby affecting transcriptional activity. They have also been shown to regulate post-translational acetylation of at least 50 non-histone proteins such as α-tubulin (see for example Kahn, N et al Biochem J 409 (2008) 581, Dokmanovic, M., et al Mol Cancer Res 5 (2007) 981).

Altering gene expression through chromatin modification can be accomplished by inhibiting HDAC enzymes. There is evidence that histone acetylation and deacetylation are mechanisms by which transcriptional regulation in a cell—a major event in cell differentiation, proliferation, and apoptosis—is achieved. It has been hypothesized that these effects occur through changes in the structure of chromatin by altering the affinity of histone proteins for coiled DNA in the nucleosome. Hypoacetylation of histone proteins is believed to increase the interaction of the histone with the DNA phosphate backbone. Tighter binding between the histone protein and DNA can render the DNA inaccessible to transcriptional regulatory elements and machinery. HDAC enzymes have been shown to catalyze the removal of acetyl groups from the ε-amino groups of lysine residues present within the N-terminal extension of core histones, thereby leading to hypoacetylation of the histones and blocking of the transcriptional machinery and regulatory elements.

Inhibition of HDAC, therefore can lead to histone deacetylase-mediated transcriptional derepression of tumor suppressor genes. For example, cells treated in culture with HDAC inhibitors have shown a consistent induction of the kinase inhibitor p21, which plays an important role in cell cycle arrest. HDAC inhibitors are thought to increase the rate of transcription of p21 by propagating the hyperacetylated state of histones in the region of the p21 gene, thereby making the gene accessible to transcriptional machinery. Further, non-histone proteins involved in the regulation of cell death and cell-cycle also undergo lysine acetylation and deacetylation by HDAC enzymes and histone acetyl transferase (HATs).

This evidence supports the use of HDAC inhibitors in treating various types of cancers. For example, vorinostat (suberoylanilide hydroxamic acid (SAHA)) has been approved by the FDA to treat cutaneous T-cell lymphoma and is being investigated for the treatment of solid and hematological tumors. Further, other HDAC inhibitors are in development for the treatment of acute myelogenous leukemia, Hodgkin's disease, myelodysplastic syndromes and solid tumor cancers.

HDAC inhibitors have also been shown to inhibit pro-inflammatory cytokines, such as those involved in autoimmune and inflammatory disorders (e.g. TNF-α). For example, the HDAC inhibitor MS275 was shown to slow disease progression and joint destruction in collagen-induced arthritis in rat and mouse models. Other HDAC inhibitors have been shown to have efficacy in treating or ameliorating inflammatory disorders or conditions in in vivo models or tests for disorders such as Crohn's disease, colitis, and airway inflammation and hyper-responsiveness. HDAC inhibitors have also been shown to ameliorate spinal cord inflammation, demyelination, and neuronal and axonal loss in experimental autoimmune encephalomyelitis (see for example Wanf, L., et al, Nat Rev Drug Disc 8 (2009) 969).

Triplet repeat expansion in genomic DNA is associated with many neurological conditions (e.g., neurodegenerative and neuromuscular diseases) including myotonic dystrophy, spinal muscular atrophy, fragile X syndrome, Huntington's disease, spinocerebellar ataxias, amyotrophic lateral sclerosis, Kennedy's disease, spinal and bulbar muscular atrophy, Friedreich's ataxia and Alzheimer's disease. Triplet repeat expansion may cause disease by altering gene expression. For example, in Huntington's disease, spinocerebellar ataxias, fragile X syndrome, and myotonic dystrophy, expanded repeats lead to gene silencing. In Friedreich's ataxia, the DNA abnormality found in 98% of FRDA patients is an unstable hyper-expansion of a GAA triplet repeat in the first intron of the frataxin gene (see Campuzano, et al., Science 271:1423 (1996)), which leads to frataxin insufficiency resulting in a progressive spinocerebellar neurodegeneration. Since they can affect transcription and potentially correct transcriptional dysregulation, HDAC inhibitors have been tested and have been shown to positively affect neurodegenerative diseases (see Herman, D., et al, Nat Chem Bio 2 551 (2006) for Friedreich's ataxia, Thomas, E. A., et al, Proc Natl Acad Sci USA 105 15564 (2008) for Huntington's disease).

HDAC inhibitors may also play a role in cognition-related conditions and diseases. It has indeed become increasingly evident that transcription is likely a key element for long-term memory processes (Alberini, C. M., Physiol Rev 89 121 (2009)) thus highlighting another role for CNS-penetrant HDAC inhibitors. Although studies have shown that treatment with non-specific HDAC inhibitors such as sodium butyrate can lead to long-term memory formation (Stefanko, D. P., et al, Proc Natl Acad Sci USA 106 9447 (2009)), little is known about the role of specific isoforms. A limited number of studies have shown that, within class I HDAC enzymes, main target of sodium butyrate, the prototypical inhibitor used in cognition studies, HDAC2 (Guan, J-S., et al, Nature 459 55 (2009)) and HDAC3 (McQuown, S. C., et al, J Neurosci 31 764 (2011)) have been shown to regulate memory processes and as such are interesting targets for memory enhancement or extinction in memory-affecting conditions such as, but not limited to, Alzheimer's disease, post-traumatic stress disorder or drug addiction.

HDAC inhibitors may also be useful to treat infectious disease such as viral infections. For example, treatment of HIV infected cells with HDAC inhibitors and anti-retroviral drugs can eradicate virus from treated cells (Blazkova, J., et al J Infect Dis. 2012 Sep 1;206(5):765-9; Archin, N. M., et al Nature 2012 Jul. 25, 487(7408):482-5).

Some prior disclosed HDAC inhibitors include a moiety of

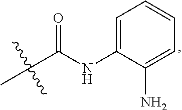 [SMILES: *C(=O)Nc1ccccc1N]

which can metabolize under physiological conditions to provide a metabolite OPD (ortho-phenylenediamine)

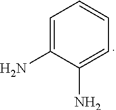

OPD is a toxic material. Thus, the need exists for HDAC inhibitors comprising a moiety of

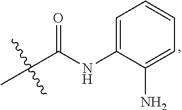 [SMILES: *C(=O)Nc1ccccc1N]

which, under physiological conditions, produce lower amounts, or substantially no amounts, of OPD.

### SUMMARY

Provided herein are compounds of formula (I), or a pharmaceutically acceptable salt thereof, and methods of using compounds of formula (I), e.g., for inhibiting HDAC (e.g., one or more of HDAC1, HDAC2, and HDAC3):

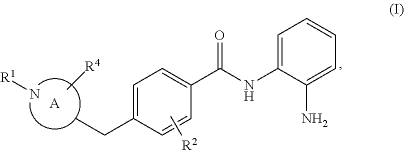

wherein ring A is a 4-7 membered monocyclic heterocycloalkyl ring or a 7-12 membered Spiro heterocycloalkyl ring, wherein ring A contains one nitrogen ring atom and optionally contains one additional ring atom independently selected from O, N, and S; R¹ is H, C₁₋₆alkyl, C₂₋₆alkenyl, C₁₋₆hydroxyalkyl, C(O)C₁₋₆alkyl, C₀₋₃alkylene-C₃₋₁₀cycloalkyl, or C₀₋₃alkylene-C₂₋₅heterocycloalkyl having 1 or 2 heteroatoms selected from O, S, N, and N(C₁₋₄alkyl); R² is H, F, Cl, or CH₃; R³ is C₁₋₃alkyl; R⁴ is H, F, or Cl; and n is 0, 1, or 2, with the proviso that (a) ring A is not morpholino or thiomorpholino; and (b) when ring A is piperazinyl, R¹ is C₂₋₆alkenyl, C₁₋₆hydroxyalkyl, C(O)C₁₋₆alkyl, C₀₋₃alkylene-C₃₋₁₀cycloalkyl, or C₀₋₃alkylene-C₂₋₅cycloheteroalkyl having 1 or 2 heteroatoms selected from O, S, N, and N(C₁₋₄alkyl).

Also provided herein are pharmaceutical compositions comprising a compound as disclosed herein, or pharmaceutically acceptable salt thereof, and a pharmaceutically acceptable carrier.

Further provided are methods of using the compounds as disclosed herein to inhibit HDAC (e.g., one or more of HDAC1, HDAC2, and HDAC3) and methods of treating conditions associated with aberrant HDAC activity by administering a compound disclosed herein to a subject suffering from such a condition.

### DETAILED DESCRIPTION

Provided herein are compounds of formula (I), pharmaceutical compositions thereof, and methods of using compounds of formula (I), e.g., for inhibiting HDAC (e.g., one or more of HDAC1, HDAC2, and HDAC3):

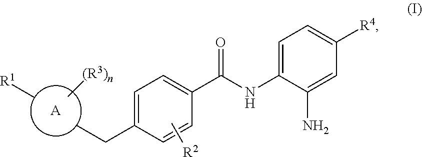

wherein ring A, R¹, R², R³, and R⁴ are defined herein.

The compounds provided herein are capable of forming low amounts of OPD under physiological conditions (e.g., a pH of about 7.2 and 37° C.). Physiological conditions as disclosed herein are intended to include a temperature of about 35 to 40° C., and a pH of about 7.0 to about 7.4 and more typically include a pH of 7.2 to 7.4 and a temperature of 36 to 38° C. in an aqueous environment. By “low amounts” of OPD, as used herein, it is intended to mean that the compounds disclosed herein generate OPD under physiological conditions for 24 hours at an amount of 30% or less. In some embodiments, the amount of OPD generated at physiological conditions for 24 hours is 25% or less, or 20% or less, or 15% or less, or 10% or less, or 5% or less, or 1% or less. The amount of OPD generated can be measured indirectly by measuring the amount of resulting acid from the amide hydrolysis of the compound. In some embodiments, the measurement of OPD generated can be performed by administration of the compound as disclosed herein to a subject, collection of plasma samples over 24 hours, and determining the amount of ODP and/or the relevant acid over that 24 hours.

### Definitions

The following definitions are used, unless otherwise described. Specific and general values listed below for radicals, substituents, and ranges, are for illustration only; they do not exclude other defined values or other values within defined ranges for the radicals and substituents.

As used herein, the term “about” preceeding a numerical value refers to a range of values ±10% of the vlaue specified.

As used herein, the term “acceptable” with respect to a formulation, composition, or ingredient, means no persistent detrimental effect on the general health of the subject being treated.

As used herein, the term “alkyl,” employed alone or in combination with other terms, refers to a saturated hydrocarbon group that may be straight-chain or branched. In some embodiments, the alkyl group contains 1 to 12, 1 to 8, or 1 to 6 carbon atoms. In certain embodiments, alkyl includes 1-6 carbon atoms (“C₁₋₆ alkyl”). In certain embodiments, alkyl includes 1-4 carbon atoms (“C₁₋₄ alkyl”). In certain embodiments alkyl includes 1-3 carbon atoms (“C₁₋₃ alkyl”).

As used herein, the term “alkylene” employed alone or in combination with other terms, refers to a divalent radical formed by removal of a hydrogen atom from alkyl. In some embodiments, the alkylene group contains 1-3 carbon atoms.

In some embodiments, alkyl includes methyl, ethyl, n-propyl, isopropyl, n-butyl, tert-butyl, isobutyl, sec-butyl; higher homologs such as 2-methyl-1-butyl, n-pentyl, 3-pentyl, n-hexyl, 1,2,2-trimethylpropyl, n-heptyl, n-octyl, and the like. In some embodiments, the alkyl moiety is methyl, ethyl, n-propyl, isopropyl, n-butyl, isobutyl, tert-butyl, n-pentyl, isopentyl, neopentyl, n-hexyl, or 2,4,4-trimethylpentyl.

As used herein, the term “alkenyl,” employed alone or in combination with other terms, refers to a saturated hydrocarbon group with at least one double bond that may be straight-chain or branched. In some embodiments, the alkenyl group contains 2 to 12, 2 to 8, or 2 to 6 carbon atoms. In certain embodiments, alkenyl include ethenyl, propenyl, 2-methylprop-1-enyl, 1-but-3-enyl, 1-pent-3-enyl, or 1-hex-5-enyl. In certain embodiments alkyl includes 2-6 carbon atoms (“C₂₋₆ alkenyl”).

As used herein, the term “cycloalkyl,” employed alone or in combination with other terms, refers to a saturated, cyclic hydrocarbon moiety of 3 to 10 carbon atoms. Cycloalkyl includes saturated or partially unsaturated rings, but does not contain an aromatic ring. In certain embodiments, cycloalkyl include a saturated, monocyclic or bicyclic hydrocarbon moiety of 3 to 10 carbon atoms. When a cycloalkyl group contains from 3-10 carbon atoms, it may be referred to herein as C₃₋₁₀ cycloalkyl. In some embodiments, the cycloalkyl group contains 3 to 7, or 3 to 6 carbon ring atoms. In some embodiments, cycloalkyl groups include cyclopropyl, cyclobutyl, cyclopentyl, cyclohexyl, and cycloheptyl. In some embodiments, cycloalkyl includes cyclopropyl, cyclopentyl, and cyclohexyl. In some embodiments, cycloalkyl includes cyclopropyl; or it includes cyclopentyl; or it includes cyclohexyl; or it includes adamantyl.

As used herein, the term “heterocycloalkyl” employed alone or in combination with other terms, refers to a saturated ring system, which has carbon ring atoms and at least one heteroatom ring atom selected from nitrogen, sulfur, and oxygen (independently selected when more than one is present), unless specified otherwise. Heterocycloalkyl includes saturated or partially unsaturated rings, but does not contain an aromatic ring. Heterocycloalkyl can include fused, bridged and spiro rings. When the heterocycloalkyl group contains more than one heteroatom, the heteroatoms may be the same or different. Heterocycloalkyl groups can include mono- or bicyclic (e.g., having 2 fused rings) ring systems. For example, a fused heterocycloalkyl group may comprise two rings that share adjacent atoms (e.g., one covalent bond). Heterocycloalkyl groups can also include bridgehead heterocycloalkyl groups. As used herein, “bridgehead heterocycloalkyl group” refers to a heterocycloalkyl moiety containing at least one bridgehead heteroatom (e.g., nitrogen or carbon). The moiety “C₂₋₅heterocycloalkyl” and the like refer to heterocycloalkyl rings having at least 2 to 5 ring carbon atoms in addition to at least 1 heteroatom. For example, a C₂ heterocycloalkyl can be a three-membered ring with 1 heteroatom in the ring and 2 carbon ring atoms, or a four-membered ring, where there are 2 carbon ring atoms and 2 heteroatoms in the ring, or a five-membered ring, where there are 2 carbon ring atoms and 3 heteroatoms in the ring.

In certain embodiments, heterocycloalkyl includes a monocyclic ring of 4 to 7 ring atoms. In certain embodiments, heterocycloalkyl includes a spiro ring system of 7 to 12 ring atoms. In certain embodiments, heterocycloalkyl includes 1, 2, or 3 nitrogen ring atoms; or 1 or 2 nitrogen ring atoms; 2 nitrogen ring atoms; or 1 nitrogen ring atom. In certain embodiments, heterocycloalkyl includes 1 nitrogen ring atom and 1 oxygen or sulfur ring atom.

In certain embodiments, heterocycloalkyl includes azetidinyl, pyrrolidinyl, 2,5-dihydro-1H-pyrrolinyl, 2,5-dihydro-1H-pyrrolyl, piperidinyl, piperazinyl, pyranyl, tetrahydropyranyl, tetrahydrothiopyranyl, 1,3-dioxinyl, 1,3-dioxanyl, 1,4-dioxinyl, 1,4-dioxanyl, perhydroazepinyl, pyrazolidinyl, imidazolinyl, imidazolidinyl, dihydropyridinyl, tetrahydropyridinyl, oxazolinyl, oxazolidinyl, isoxazolidinyl, thiazolinyl, thiazolidinyl, quinuclidinyl, isothiazolidinyl, octahydroindolyl, octahydroisoindolyl, decahydroisoquinolyl, tetrahydrofuryl, 2-azaspiro[3.3]heptanyl, 7-azabicyclo[2.2.1]heptanyl, and 8-azabicyclo[3.2.1]octanyl. In some embodiments, the heterocycloalkyl comprises piperidinyl, piperazinyl, azetidinyl, azepanyl, or diazepanyl, e.g., piperidinyl or piperazinyl. In some embodiments, the heterocycloalkyl comprises piperidinyl, piperazinyl, azetidinyl, azepanyl, pyrrolidinyl or diazepanyl. Specifically contemplated spiro heterocycloalkyl groups include azetidinyl ring spiro fused to another azetidinyl ring or a piperidinyl ring, or a piperazinyl ring, and an oxetanyl ring spiro fused to an azetidinyl ring or a piperidinyl ring or a piperazinyl ring, or a cyclohexyl ring spiro fused to an azetidinyl ring or a piperidinyl ring or a piperazinyl ring.

As used herein, the term “hydroxyalkyl” and the like employed alone or in combination with other terms, refers to an alkyl group having at least one hydroxy group. In certain embodiments, hydroxyalkyl refers to an alkyl group having 1 hydroxy group. In certain embodiments, hydroxyalkyl refers to an alkyl group having 1, 2, or 3 hydroxy group.

The term “subject” refers to a mammal, such as a mouse, guinea pig, rat, dog, or human. In certain embodiments, mammal include sheep, goat, horse, cat, rabbit, monkey, or cow. The terms “subject” and “patient” are used interchangeably. In certain embodiments, the subject is a human; or the subject is a human adult; or the subject is a human child.

“Treat,” “treating,” and “treatment,” in the context of treating a disease or disorder, are meant to include alleviating or abrogating a disorder, disease, or condition, or one or more of the symptoms associated with the disorder, disease, or condition; or to slowing the progression, spread or worsening of a disease, disorder or condition or of one or more symptoms thereof. Often, the beneficial effects that a subject derives from a therapeutic agent do not result in a complete cure of the disease, disorder or condition.

Although methods and materials similar or equivalent to those described herein can be used in practice or testing, suitable methods and materials are described below. All publications, patent applications, patents, and other references mentioned herein are incorporated by reference in their entirety. In embodiment of conflict, the present specification, including definitions, will control.

Compounds of formula (I)

Compounds of formula (I) are provided herein:

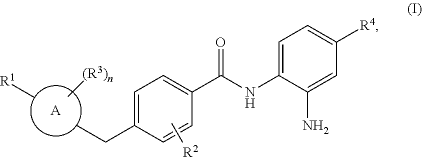

ring A is a 4-7 membered monocyclic heterocycloalkyl ring or a 7-12 membered spiro heterocycloalkyl ring, wherein ring A contains one nitrogen ring atom and optionally contains one additional ring atom independently selected from O, N, and S; R¹ is H, C₁₋₆alkyl, C₂₋₆alkenyl, C₁₋₆hydroxyalkyl, C(O)C₁₋₆alkyl, C₀₋₃alkylene-C₃₋₁₀cycloalkyl, or C₀₋₃alkylene-C₂₋₅heterocycloalkyl having 1 or 2 heteroatoms selected from O, S, N, and N(C₁₋₄alkyl); R² is H, F, Cl, or CH₃; R³ is C₁₋₃alkyl; R⁴ is H, F, or Cl; and n is 0, 1, or 2, with the proviso that (a) ring A is not morpholino or thiomorpholino; and (b) when ring A is piperazinyl, R¹ is C₂₋₆alkenyl, C₁₋₆hydroxyalkyl, C(O)C₁₋₆alkyl, C₀₋₃alkylene-C₃₋₁₀cycloalkyl, or C₀₋₃alkylene-C₂₋₅cycloheteroalkyl having 1 or 2 heteroatoms selected from O, S, N, and N(C₁₋₄alkyl). In some embodiments, R¹ is H, C₁₋₆alkyl, C₃₋₆hydroxyalkyl, C₃₋₆alkenyl, or C₁₋₂alkylene-C₃₋₁₀cycloalkyl; R² is H; R³, if present, is CH₃, and R⁴ is H. In certain embodiments, R¹ is C₁₋₆alkyl, C₃₋₆hydroxyalkyl, or C₁₋₂alkylene-C₃₋₁₀cycloalkyl; R² is H; R³, if present, is CH₃, and R⁴ is F.

In some some cases, the compound of formula (I) has the following characteristics: ring A is piperidinyl, azetidinyl, azepanyl, diazepanyl, pyrrolidinyl,

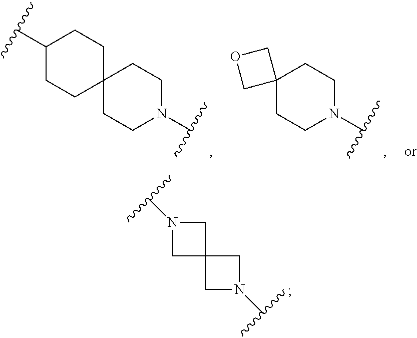 [SMILES: *C1CCC2(CC1)CCN(*)CC2 ; *N1CCC2(CC1)COC2 ; *N1CC2(C1)CN(*)C2]

R¹ is H, C₁₋₆alkyl, C₂₋₆alkenyl, C₁₋₆hydroxyalkyl, C(O)C₁₋₆alkyl, C₀₋₃alkylene-C₃₋₁₀cycloalkyl, or C₀₋₃alkylene-C₂₋₅heterocycloalkyl having 1 or 2 heteroatoms selected from O, S, N, and N(C₁₋₄alkyl); R² is H, F, Cl, or CH₃; R³ is C₁₋₃alkyl; R⁴ is H, F, or Cl; and n is 0, 1, or 2.

In some some cases, the compound of formula (I) has the following characteristics: ring A is piperidinyl, azetidinyl, azepanyl, diazepanyl, pyrrolidinyl,

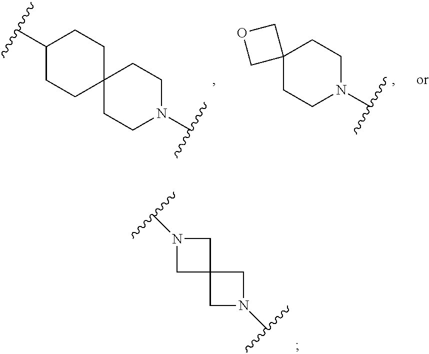 [SMILES: *C1CCC2(CC1)CCN(*)CC2 ; *N1CCC2(CC1)COC2 ; *N1CC2(C1)CN(*)C2]

R¹ is H, C₁₋₆alkyl, C₂₋₆alkenyl, C₁₋₆hydroxyalkyl, or C₀₋₃alkylene-C₃₋₁₀cycloalkyl; R² is H; R³ is C₁₋₃alkyl; R⁴ is H or F; and n is 0, 1, or 2.

In some cases, the compound of formula (I) has the following characteristics: ring A is piperazinyl; R¹ is C₂₋₆alkenyl, C₁₋₆hydroxyalkyl, or C₀₋₃alkylene-C₃₋₁₀cycloalkyl; R² is H, F, Cl, or CH₃; R³ is C₁₋₃alkyl; R⁴ is H or F; and n is 0, 1, or 2. In some cases, the compound of formula (I) has the following characteristics: ring A is piperazinyl; R¹ is C₁₋₆hydroxyalkyl or C₀₋₃alkylene-C₃₋₁₀cycloalkyl; R² is H; R³ is C₁₋₃alkyl; R⁴ is H; and n is 0, 1, or 2.

In various embodiments, ring A is a 4-7 membered monocyclic heterocycloalkyl ring or a 7-12 membered spiro heterocycloalkyl ring, wherein ring A contains one nitrogen ring atom and optionally contains one additional ring atom independently selected from O, N, and S. In various embodiments, ring A is a 4-7 membered monocyclic heterocycloalkyl ring or a 7-12 membered spiro heterocycloalkyl ring, wherein ring A contains one nitrogen ring atom and optionally contains one additional nitrogen ring atom. In various embodiments, ring A is a 4-7 membered monocyclic heterocycloalkyl ring or a 7-12 membered spiro heterocycloalkyl ring, wherein ring A contains one nitrogen ring atom and optionally contains one oxygen ring atom. In various embodiments, ring A is a 4-7 membered monocyclic heterocycloalkyl ring or a 7-12 membered spiro heterocycloalkyl ring, wherein ring A contains one nitrogen ring atom and optionally contains one sulfur ring atom. In various embodiments, ring A is a 7-12 membered spiro heterocycloalkyl ring containing one or two nitrogen ring atoms or one nitrogen ring atom and one oxygen ring atom. In various embodiments, ring A is a 7-12 membered spiro heterocycloalkyl ring containing one or two nitrogen ring atoms. In various embodiments, ring A is a 7-12 membered spiro heterocycloalkyl ring containing one nitrogen ring atom. In various embodiments, ring A is a 7-12 membered spiro heterocycloalkyl ring containing two nitrogen ring atoms. In various embodiments, ring A is a 7-12 membered spiro heterocycloalkyl ring containing one nitrogen ring atom and one oxygen ring atom. In various cases, ring A is a 4-7 membered monocyclic heterocycloalkyl ring containing one or two nitrogen ring atoms. In various cases, ring A is a 4-7 membered monocyclic heterocycloalkyl ring containing one nitrogen ring atom. In various cases, ring A is a 4-7 membered monocyclic heterocycloalkyl ring containing two nitrogen ring atoms. Some specific ring A moieties contemplated include piperidinyl, piperazinyl, azetidinyl, azepanyl, and diazepanyl. Some specific ring A moieties contemplated include piperidinyl, piperazinyl, azetidinyl, azepanyl, diazepanyl, and pyrrolidinyl. In certain embodiments, ring A is piperidinyl, piperazinyl, or azetidinyl. In certain embodiments, ring A is piperidinyl, piperazinyl, azetidinyl, or pyrrolidinyl. In certain embodiments, ring A is piperidinyl, pyrrolidinyl, or azetidinyl. In certain embodiments, ring A is azetidinyl, azepanyl, or diazepanyl. In certain embodiments, ring A is azepanyl or diazepanyl. In certain embodiments, ring A is piperidinyl. In certain embodiments, ring A is piperazinyl. In certain embodiments, ring A is azetidinyl. In certain embodiments, ring A is azepanyl. In certain embodiments, ring A is diazepanyl. In certain embodiments, ring A is pyrrolidinyl. Some specific spiro ring A moieties contemplated include azetidinyl ring spiro fused to another azetidinyl ring or a piperidinyl ring, or a piperazinyl ring, and an oxetanyl ring spiro fused to an azetidinyl ring or a piperidinyl ring or a piperazinyl ring, or a cyclohexyl ring spiro fused to an azetidinyl ring or a piperidinyl ring or a piperazinyl ring. In some cases, ring A can be piperidinyl or piperazinyl. In various cases, ring A is selected from the group consisting of:

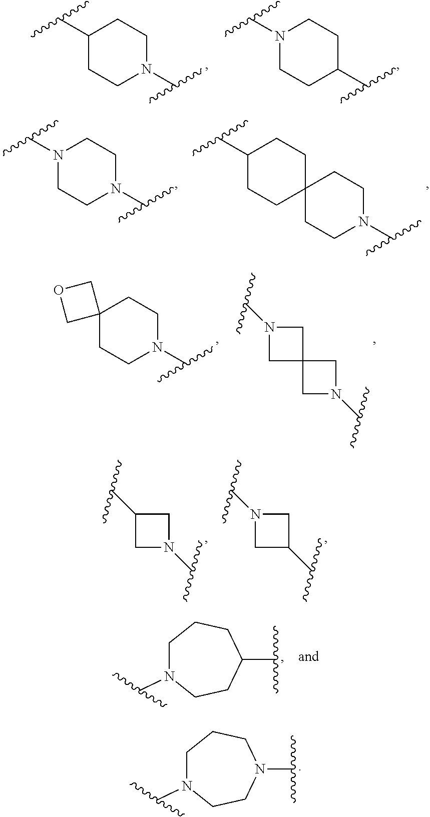

In various cases, ring A is selected from the group consisting of:

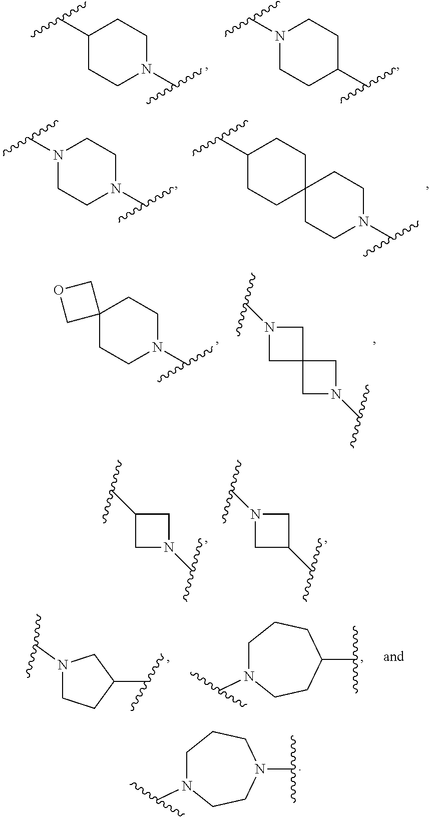

R¹ can be H, C₁₋₆alkyl, C₂₋₆alkenyl, C₁₋₆hydroxyalkyl, C(O)C₁₋₆alkyl, C₀₋₃alkylene-C₃₋₁₀cycloalkyl, or C₀₋₃alkylene-C₂₋₅heterocycloalkyl having 1 or 2 heteroatoms selected from O, S, N, and N(C₁₋₄alkyl). In some cases, R¹ is H. In some cases, R¹ is C₁₋₆alkyl (e.g., methyl, isopropyl, sec-butyl, or CH₂C(CH₃)₃). In some cases, R¹ is methyl, ethyl, propyl, isopropyl, butyl, sec-butyl, or CH₂C(CH₃)₃). In some cases, R¹ is methyl or neopentyl. In some cases R¹ is methyl. In some cases R¹ is neopentyl. In some cases, R¹ is C₁₋₆hydroxyalkyl (e.g.,

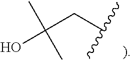 [SMILES: *CC(C)(C)O]

In some cases R¹ is

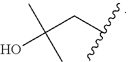 [SMILES: *CC(C)(C)O]

In some cases, R¹ is C₃₋₁₀cycloalkyl or C₁₋₃alkylene-C₃₋₁₀cycloalkyl, e.g., the cycloalkyl group is cyclopropyl or C₁₀cycloalkyl, i.e., adamantyl. In some cases, R¹ is C₁₋₃alkylene-C₃₋₁₀cycloalkyl, e.g.

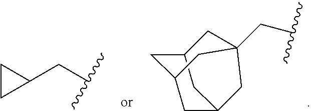 [SMILES: *CC1CC1 ; *CC12CC3CC(CC(C3)C1)C2]

In some cases R¹ is

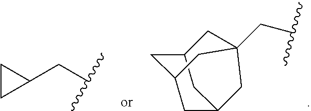 [SMILES: *CC1CC1 ; *CC12CC3CC(CC(C3)C1)C2]

In some cases R¹ is

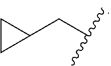 [SMILES: *CC1CC1]

In some cases R¹ is

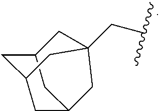 [SMILES: *CC12CC3CC(CC(C3)C1)C2]

In certain embodiments, R¹ is C₂₋₆alkenyl. In certain embodiments, R¹ is

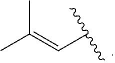 [SMILES: *C=C(C)C]

In some cases, the compound of formula (I) has the following characteristics:

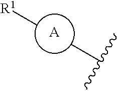

is selected from the group consisting of

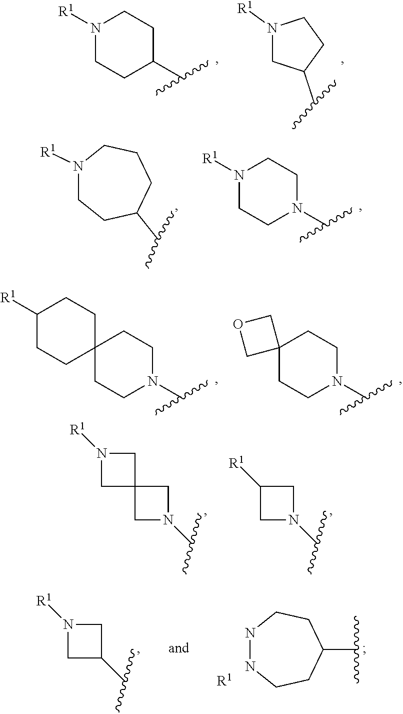

R¹ is selected from the group consisting of H, CH₃,

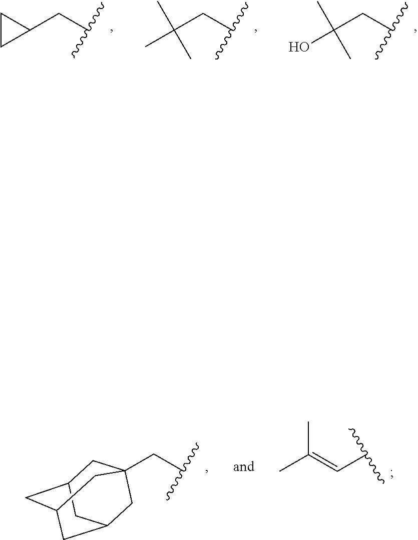

R² is H, F, Cl, or CH₃; R³ is CH₃, R⁴ is H or F; and n is 0, 1, or 2.

In some cases, the compound of formula (I) has the following characteristics:

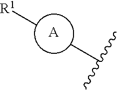

is selected from the group consisting of

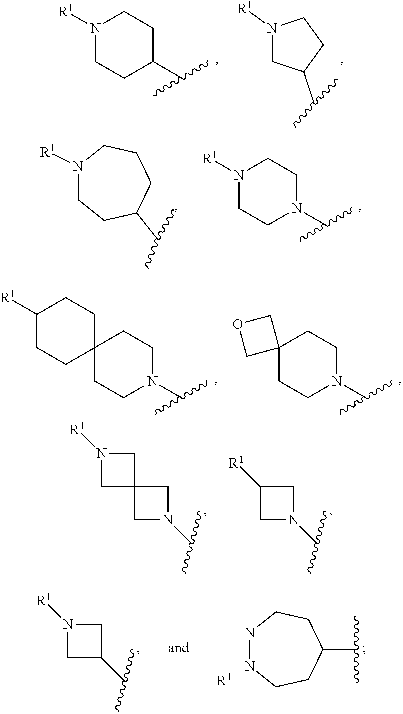

R¹ is selected from the group consisting of H, CH₃,

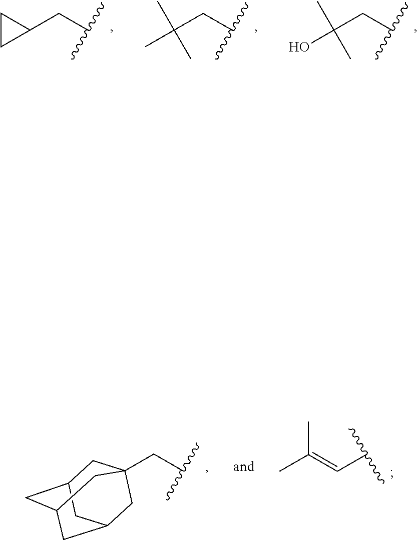

R² is H; R³ is CH₃; R⁴ is H or F; and n is 0, 1, or 2. In certain embodiments, n is 0. In certain embodiments n is 1. In certain embodiments, n is 2.

In some cases, the compound of formula (I) has the following characteristics:

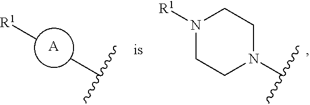

R¹ is selected from the group consisting of

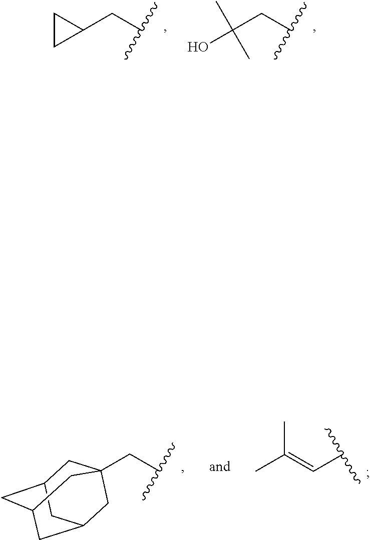

R² is H, F, Cl, or CH₃; R³ is CH₃, R⁴ is H or F; and n is 0, 1, or 2.

In some cases, the compound of formula (I) has the following characteristics:

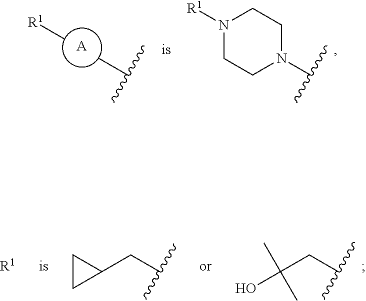

R² is H; R³ is CH₃; R⁴ is H; and n is 0, 1, or 2.

In some cases, the compound of formula (I) has the following characteristics: ring A is piperidinyl; R¹ is H, C₁₋₆alkyl, C₂₋₆alkenyl, C₁₋₆hydroxyalkyl, or C₀₋₃alkylene-C₃₋₁₀cycloalkyl; R² is H, F, Cl, or CH₃; R³ is C₁₋₃alkyl; R⁴ is H, F, or Cl; and n is 0, 1, or 2. In some cases, the compound of formula (I) has the following characteristics: ring A is piperidinyl; R¹ is H, C₁₋₆alkyl, C₂₋₆alkenyl, C₁₋₆hydroxyalkyl, or C₀₋₃alkylene-C₃₋₁₀cycloalkyl; R² is H; R³ is C₁₋₃alkyl; R⁴ is H or F; and n is 0, 1, or 2.

In some cases, the compound of formula (I) has the following characteristics: ring A is azetidinyl; R¹ is H, C₁₋₆alkyl, C₂₋₆alkenyl, C₁₋₆hydroxyalkyl, or C₀₋₃alkylene-C₃₋₁₀cycloalkyl; R² is H, F, Cl, or CH₃; R³ is C₁₋₃alkyl; R⁴ is H, F, or Cl; and n is 0, 1, or 2. In some cases, the compound of formula (I) has the following characteristics: ring A is azetidinyl; R¹ is H or C₀₋₃alkylene-C₃₋₁₀cycloalkyl; R² is H; R³ is C₁₋₃alkyl; R⁴ is H; and n is 0, 1, or 2. In some cases, the compound of formula (I) has the following characteristics: ring A is azetidinyl; R¹ is H or C₀₋₃alkylene-C₃₋₁₀cycloalkyl; R² is H; R⁴ is H; and n is 0.

In various cases, R² is H. In some cases, R² is F. In some cases, R² is Cl. In some cases, R² is CH₃.

For compounds of formula (I), n can be 0. In certain embodiments, when n is 1 or 2, R³ is C₁₋₃alkyl, and can be, e.g., CH₃. In certain embodiments, when n is 2, each R³ can be substituted at the same atom of ring A, or at different atoms of ring A. In certain embodiments, when n is 2, each R³ is CH₃ and each R³ is substituted at the same atom of ring A. In certain embodiments, when n is 2, each R³ is CH₃ and each R³ is substituted at different atoms of ring A.

R⁴ can be H, or can be F, or can be Cl. In various cases, R⁴ is H or F. In some cases, R⁴ is H. In some cases, R⁴ is F.

Some specific compounds contemplated herein are listed in Table 1.

TABLE 1

| Ex | Structure |
|---|---|
| 146 | 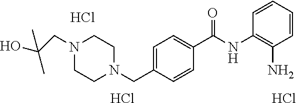 [SMILES: CC(C)(O)CN1CCN(Cc2ccc(C(=O)Nc3ccccc3N)cc2)CC1 ; Cl ; Cl ; Cl] |
| 147 | 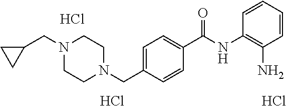 [SMILES: Nc1ccccc1NC(=O)c1ccc(CN2CCN(CC3CC3)CC2)cc1 ; Cl ; Cl ; Cl] |
| 171 | 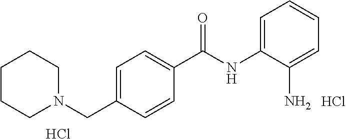 [SMILES: Nc1ccccc1NC(=O)c1ccc(CN2CCCCC2)cc1 ; Cl ; Cl] |
| 172 | 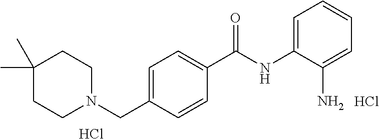 [SMILES: CC1(C)CCN(Cc2ccc(C(=O)Nc3ccccc3N)cc2)CC1 ; Cl ; Cl] |
| 174 | 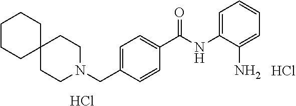 [SMILES: Nc1ccccc1NC(=O)c1ccc(CN2CCC3(CCCCC3)CC2)cc1 ; Cl ; Cl] |
| 354 | 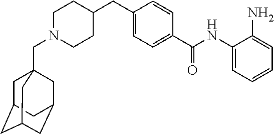 [SMILES: Nc1ccccc1NC(=O)c1ccc(CC2CCN(CC34CC5CC(CC(C5)C3)C4)CC2)cc1] |
| 175 | 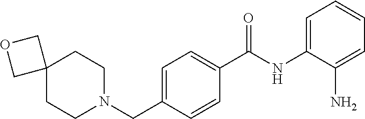 [SMILES: Nc1ccccc1NC(=O)c1ccc(CN2CCC3(CC2)COC3)cc1] |
| 241 | 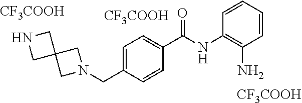 [SMILES: "CF3COOH" ; Nc1ccccc1NC(=O)c1ccc(CN2CC3(CNC3)C2)cc1 ; "CF3COOH" ; "CF3COOH"] |
| 238 | 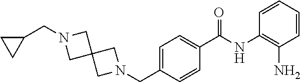 [SMILES: Nc1ccccc1NC(=O)c1ccc(CN2CC3(C2)CN(CC2CC2)C3)cc1] |
| 176 | 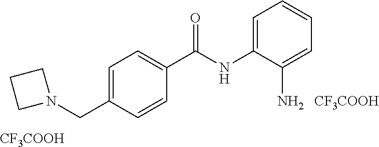 |
| 161 | |
| 163 | |
| 169 | |
| 162 | |
| 356 | 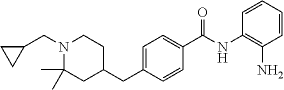 [SMILES: CC1(C)CC(Cc2ccc(C(=O)Nc3ccccc3N)cc2)CCN1CC1CC1] |
| 357 | 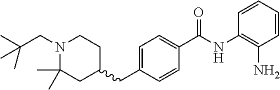 [SMILES: CC(C)(C)CN1CCC(Cc2ccc(C(=O)Nc3ccccc3N)cc2)CC1(C)C] |
| 379 | 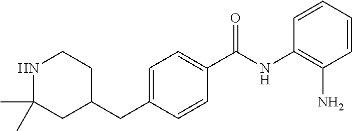 [SMILES: CC1(C)CC(Cc2ccc(C(=O)Nc3ccccc3N)cc2)CCN1] |
| 181 | 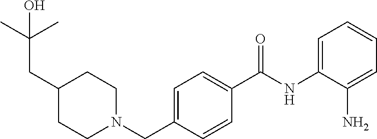 [SMILES: CC(C)(O)CC1CCN(Cc2ccc(C(=O)Nc3ccccc3N)cc2)CC1] |
| 472 | 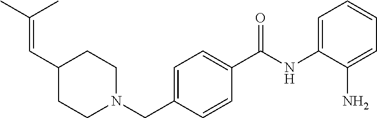 [SMILES: CC(C)=CC1CCN(Cc2ccc(C(=O)Nc3ccccc3N)cc2)CC1] |
| 485 | 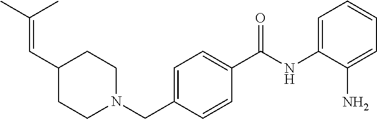 [SMILES: CC(C)=CC1CCN(Cc2ccc(C(=O)Nc3ccccc3N)cc2)CC1] |
| 486 | 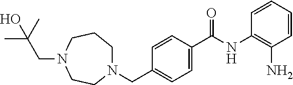 [SMILES: CC(C)(O)CN1CCCN(Cc2ccc(C(=O)Nc3ccccc3N)cc2)CC1] |
| 477 | |
| 478 | |
| 480 | 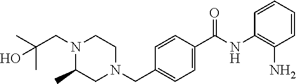 [SMILES: C[C@@H]1CN(Cc2ccc(C(=O)Nc3ccccc3N)cc2)CCN1CC(C)(C)O] |
| 479 | 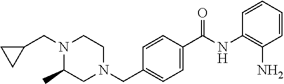 [SMILES: C[C@@H]1CN(Cc2ccc(C(=O)Nc3ccccc3N)cc2)CCN1CC1CC1] |
| 482 | 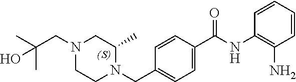 |
| 481 | 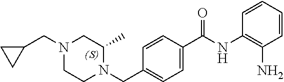 |
| 489 | 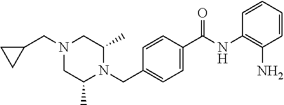 [SMILES: C[C@@H]1CN(CC2CC2)C[C@H](C)N1Cc1ccc(C(=O)Nc2ccccc2N)cc1] |
| 490 | 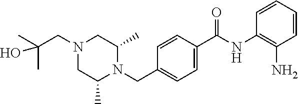 [SMILES: C[C@@H]1CN(CC(C)(C)O)C[C@H](C)N1Cc1ccc(C(=O)Nc2ccccc2N)cc1] |
| 491 | 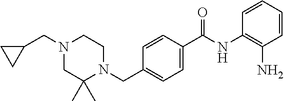 [SMILES: CC1(C)CN(CC2CC2)CCN1Cc1ccc(C(=O)Nc2ccccc2N)cc1] |
| 478-I | 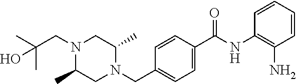 [SMILES: C[C@@H]1CN(Cc2ccc(C(=O)Nc3ccccc3N)cc2)[C@@H](C)CN1CC(C)(C)O] |
| 478-II | 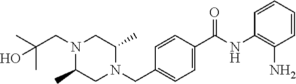 [SMILES: C[C@@H]1CN(Cc2ccc(C(=O)Nc3ccccc3N)cc2)[C@@H](C)CN1CC(C)(C)O] |
| 484 | 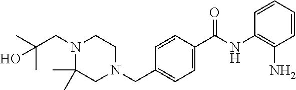 [SMILES: CC(C)(O)CN1CCN(Cc2ccc(C(=O)Nc3ccccc3N)cc2)CC1(C)C] |
| 492 | 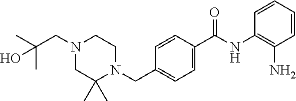 [SMILES: CC(C)(O)CN1CCN(Cc2ccc(C(=O)Nc3ccccc3N)cc2)C(C)(C)C1] |
| 483 | 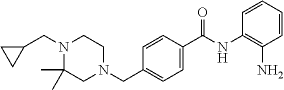 [SMILES: CC1(C)CN(Cc2ccc(C(=O)Nc3ccccc3N)cc2)CCN1CC1CC1] |
| 477-II | 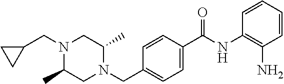 [SMILES: C[C@@H]1CN(Cc2ccc(C(=O)Nc3ccccc3N)cc2)[C@@H](C)CN1CC1CC1] |
| 477-I | 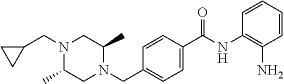 [SMILES: C[C@@H]1CN(CC2CC2)[C@@H](C)CN1Cc1ccc(C(=O)Nc2ccccc2N)cc1] |
| 487 | 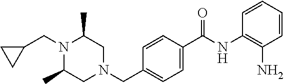 [SMILES: C[C@@H]1CN(Cc2ccc(C(=O)Nc3ccccc3N)cc2)C[C@H](C)N1CC1CC1] |
| 488 | 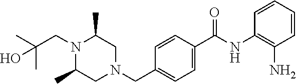 [SMILES: C[C@@H]1CN(Cc2ccc(C(=O)Nc3ccccc3N)cc2)C[C@H](C)N1CC(C)(C)O] |

Some specific compounds contemplated include those listed in Table 2.

TABLE 2

| | |
|---|---|
| 555 | 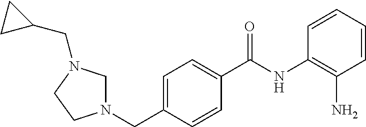 [SMILES: Nc1ccccc1NC(=O)c1ccc(CN2CCN(CC3CC3)C2)cc1] |
| 556 | 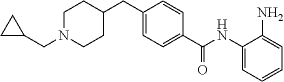 [SMILES: Nc1ccccc1NC(=O)c1ccc(CC2CCN(CC3CC3)CC2)cc1] |

In certain embodiments, the compound or salt thereof is selected from Table 1. In certain embodiments, the compound or salt thereof is selected from Table 2. In certain embodiments the compound or salt thereof is selected from Table 1 and Table 2.

In certain embodiments, the compound or salt thereof is selected from the group consisting of compounds 485, 486, 479, 480, 483, 484, 482, 481, 489, 490, 491, 492, 487, 488, 477, 477-I, 477-II, 478, 478-I, 478-II, 356, 359, 357, 379, 181, 472, 238, 241, 176, 171, 172, 174, 175, 354, 169, 161, 162, 163, 146, 147, 555, and 556, or a single stereoisomer or mixture of stereoisomers thereof.

Compounds of formula (I) described herein may contain one or more asymmetric centers and thus occur as racemates and racemic mixtures, single enantiomers, individual diastereomers and diastereomeric mixtures. While shown without respect to the stereochemistry in formula (I), the present disclosure includes such optical isomers (enantiomers) and diastereomers; as well as the racemic and resolved, enantiomerically pure R and S stereoisomers; as well as other mixtures of the R and S stereoisomers and pharmaceutically acceptable salts thereof. The use of these compounds is intended to cover the racemic mixture or either of the chiral enantiomers.

One skilled in the art will also recognize that it is possible for tautomers to exist for the compounds described herein. The disclosure includes all such tautomers even though not shown in the formulas herein. All such isomeric forms of such compounds are expressly included in the present disclosure.

Optical isomers can be obtained in pure form by standard procedures known to those skilled in the art, and include, but are not limited to, diastereomeric salt formation, kinetic resolution, and asymmetric synthesis. See, for example, Jacques, et al., Enantiomers, Racemates and Resolutions (Wiley Interscience, New York, 1981); Wilen, S. H., et al., Tetrahedron 33:2725 (1977); Eliel, E. L. Stereochemistry of Carbon Compounds (McGraw-Hill, NY, 1962); Wilen, S. H. Tables of Resolving Agents and Optical Resolutions p. 268 (E. L. Eliel, Ed., Univ. of Notre Dame Press, Notre Dame, Ind. 1972), each of which is incorporated herein by reference in their entireties. It is also understood that this disclosure encompasses all possible regioisomers, and mixtures thereof, which can be obtained in pure form by standard separation procedures known to those skilled in the art, and include, but are not limited to, column chromatography, thin-layer chromatography, and high-performance liquid chromatography.

Compounds described herein can also include all isotopes of atoms occurring in the intermediates or final compounds. Isotopes include those atoms having the same atomic number but different mass numbers. For example, isotopes of hydrogen include tritium and deuterium, preferably deuterium.

The compounds described herein also include pharmaceutically acceptable salts of the compounds disclosed herein. As used herein, the term “pharmaceutically acceptable salt” refers to a salt formed by the addition of a pharmaceutically acceptable acid or base to a compound disclosed herein. As used herein, the phrase “pharmaceutically acceptable” refers to a substance that is acceptable for use in pharmaceutical applications from a toxicological perspective and does not adversely interact with the active ingredient. Pharmaceutically acceptable salts, including mono- and bi- salts, include, but are not limited to, those derived from organic and inorganic acids such as, but not limited to, acetic, lactic, citric, cinnamic, tartaric, succinic, fumaric, maleic, malonic, mandelic, malic, oxalic, propionic, hydrochloric, hydrobromic, phosphoric, nitric, sulfuric, glycolic, pyruvic, methanesulfonic, ethanesulfonic, toluenesulfonic, salicylic, benzoic, and similarly known acceptable acids. Lists of suitable salts are found in Remington's Pharmaceutical Sciences, 17th ed., Mack Publishing Company, Easton, Pa., 1985, p. 1418; Journal of Pharmaceutical Science, 66, 2 (1977); and “Pharmaceutical Salts: Properties, Selection, and Use A Handbook; Wermuth, C. G. and Stahl, P. H. (eds.) Verlag Helvetica Chimica Acta, Zurich, 2002 [ISBN 3-906390-26-8] each of which is incorporated herein by reference in their entireties.

### Methods of Use

All the compounds and pharmaceutical compositions provided herein can be used in any of the methods provided herein.

Provided herein are methods of inhibiting one or more HDAC enzymes (e.g., HDAC1 or HDAC2; e.g., HDAC3) or more than one HDAC (e.g., HDAC1 and HDAC2; e.g., HDAC1 and HDAC3; e.g., HDAC2 or HDAC3; e.g., HDAC1, HDAC2, and HDAC3) using a compound or a salt thereof as disclosed herein. In some embodiments, the methods can include contacting one or more HDAC enzymes (e.g., HDAC1 or HDAC2; e.g., HDAC3) in a sample with a compound or a salt thereof as disclosed herein. In other embodiments, the methods can include administering a compound or a salt thereof as disclosed herein to a subject (e.g., a mammal, such as a human).

A histone deacetylase (HDAC), as described herein, can be any polypeptide having features characteristic of polypeptides that catalyze the removal of the acetyl group (deacetylation) from acetylated target proteins. Features characteristic of HDAC enzymes are known in the art (see, for example, Finnin et al., 1999, Nature, 401:188). Thus, an HDAC enzyme can be a polypeptide that represses gene transcription by deacetylating the ε-amino groups of conserved lysine residues located at the N-termini of histones, e.g., H3, H4, H2A, and H2B, which form the nucleosome. HDAC enzymes also deacetylate other proteins such as p53, E2F, α-tubulin, and MyoD (see, for example, Annemieke et al., 2003, Biochem. J., 370:737). HDAC enzymes can also be localized to the nucleus and certain HDAC enzymes can be found in both the nucleus and also the cytoplasm.

Compounds described herein can interact with any HDAC enzyme. In some embodiments, the compounds described herein will have at least about 2-fold (e.g., at least about 5-fold, 10-fold, 15-fold, or 20-fold) greater activity to inhibit one or more class I HDAC enzymes (e.g., HDAC1, HDAC2, or HDAC3) as compared to one or more other HDAC enzymes (e.g., one or more HDAC enzymes of class IIa, IIb, or IV).

In some embodiments, a compound or a salt thereof as disclosed herein selectively inhibits HDAC3, e.g., selectively inhibits HDAC3 over HDAC1 and HDAC2 (e.g. exhibiting 5-fold or greater selectivity, e.g. exhibiting 25-fold or greater selectivity). While not wishing to be bound by theory, it is believed that HDAC3-selective inhibitors can increase expression of frataxin, and can therefore be useful in the treatment of neurological conditions (e.g., neurological conditions associated with reduced frataxin expression, such as Friedreich's ataxia). It is also believed that HDAC3 inhibition plays an important role in memory consolidation (McQuown S C et al, J Neurosci 31 764 (2011)). Selective inhibitors of HDAC3 provide advantages for treatment of neurological conditions over the use of broad-spectrum HDAC inhibitors by reducing toxicities associated with inhibition of other HDAC enzymes. Such specific HDAC3 inhibitors can provide a higher therapeutic index, resulting in better tolerance by patients during chronic or long-term treatment.

In some further embodiments, compounds selectively inhibit HDAC1 and/or HDAC2 (e.g. exhibiting 5-fold or greater selectivity, e.g. exhibiting 25-fold or greater selectivity). Inhibition of HDAC1 and/or 2 can be useful in treating cancer, or another disease as disclosed herein.

In some embodiments, a compound or a salt thereof as disclosed herein exhibits enhanced brain penetration. For example, brain/plasma ratios of greater than about 0.25 (e.g., greater than about 0.50, greater than about 1.0, greater than about 1.5, or greater than about 2.0) are observed when rats, mice, dogs, or monkeys are dosed with some of the compounds disclosed herein. In some embodiments, a compound or a salt thereof as disclosed herein selectively inhibits HDAC3, e.g., selectively inhibits HDAC3 over HDAC1 and HDAC2 (e.g., exhibiting 5-fold or greater selectivity, e.g. exhibiting 25-fold or greater selectivity) and exhibits enhanced brain penetration. In some embodiments, a compound described herein selectively inhibits HDAC1 and/or HDAC2, e.g., selectively inhibit HDAC1 and/or HDAC2 over HDAC3 (e.g., exhibiting 5-fold or greater selectivity, e.g. exhibiting 25-fold or greater selectivity) and exhibits enhanced brain penetration.

Compounds with enhanced brain penetration are suitable for therapies targeting the brain (e.g., neurological conditions such as Friedreich's ataxia, myotonic dystrophy, spinal muscular atrophy, fragile X syndrome, Huntington's disease, spinocerebellar ataxia, Kennedy's disease, amyotrophic lateral sclerosis, spinal and bulbar muscular atrophy, and Alzheimer's disease; a memory impairment condition, frontotemportal dementia; post-traumatic stress disorder; a drug addiction).

Provided herein are methods of treating a disease or disorder mediated by HDAC in a subject (e.g., a mammal, such as a human) in need thereof, which include administering a compound or a salt thereof as disclosed herein to the subject.

Further provided herein are methods of preventing a disease or disorder mediated by HDAC in a subject (e.g., a mammal, such as a human) in need thereof. Prevention can include delaying the onset of or reducing the risk of developing, a disease, disorder, or condition or symptoms thereof.

The disclosure further provides a method of treating a cancer in patient in need thereof, comprising administering a therapeutically effective amount of an HDAC inhibitor as described herein, or salt thereof. In some embodiments, the cancer is a solid tumor, neoplasm, carcinoma, sarcoma, leukemia, or lymphoma. In some embodiments, leukemias include acute leukemias and chronic leukemias such as acute lymphocytic leukemia (ALL), acute myeloid leukemia, chronic lymphocytic leukemia (CLL), chronic myelogenous leukemia (CML) and Hairy Cell Leukemia; lymphomas such as cutaneous T-cell lymphomas (CTCL), noncutaneous peripheral T-cell lymphomas, lymphomas associated with human T-cell lymphotrophic virus (flTLV) such as adult T-cell leukemia/lymphoma (ATLL), Hodgkin's disease and non-Hodgkin's lymphomas, large-cell lymphomas, diffuse large B-cell lymphoma (DLBCL); Burkitt's lymphoma; primary central nervous system (CNS) lymphoma; multiple myeloma; childhood solid tumors such as brain tumors, neuroblastoma, retinoblastoma, Wilm's tumor, bone tumors, and soft-tissue sarcomas, common solid tumors of adults such as head and neck cancers (e.g., oral, laryngeal and esophageal), genitor-urinary cancers (e.g., prostate, bladder, renal, uterine, ovarian, testicular, rectal and colon), lung cancer, breast cancer.

In some embodiments, the cancer is (a) Cardiac: sarcoma (angiosarcoma, fibrosarcoma, rhabdomyosarcoma, liposarcoma), myxoma, rhabdomyoma, fibroma, lipoma and teratoma; (b) Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; (c) Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma); (d) Genitourinary tract: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); (e) Liver: hepatoma (hepatocellular carcinoma), cholangiocarcinoma, hepatoblastoma, angiosarcoma, hepatocellular adenoma, hemangioma; (f) Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochrondroma (osteocartilaginous exostoses), benign chondroma, chondroblastoma, chondromyxofibroma, osteoid osteoma and giant cell tumors; (g) Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord (neurofibroma, meningioma, glioma, sarcoma); (h) Gynecological: uterus (endometrial carcinoma), cervix (cervical carcinoma, pre-tumor cervical dysplasia), ovaries (ovarian carcinoma, serous cystadenocarcinoma, mucinous cystadenocarcinoma), unclassified carcinoma (granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma), embryonal rhabdomyosarcoma, fallopian tubes (carcinoma); (i) Hematologic: blood (myeloid leukemia [acute and chronic], acute lymphoblastic leukemia, chronic lymphocytic leukemia, myeloproliferative diseases, multiple myeloma, myelodysplastic syndrome), Hodgkin's disease, non-Hodgkin's lymphoma (malignant lymphoma); (j) Skin: malignant melanoma, basal cell carcinoma, squamous cell carcinoma, Karposi's sarcoma, moles dysplastic nevi, lipoma, angioma, dermatofibroma, keloids, psoriasis; and (k) Adrenal glands: neuroblastoma conditions.

In another aspect, provided is a method of treating an inflammatory disorder in patient in need thereof, comprising administering a therapeutically effective amount of a compound as described herein, or salt thereof. In some embodiments, the inflammatory disorder is an acute and chronic inflammatory disease, autoimmune disease, allergic disease, disease associated with oxidative stress, and diseases characterized by cellular hyperproliferation. Non-limiting examples are inflammatory conditions of a joint including rheumatoid arthritis (RA) and psoriatic arthritis; inflammatory bowel diseases such as Crohn's disease and ulcerative colitis; spondyloarthropathies; scleroderma; psoriasis (including T-cell mediated psoriasis) and inflammatory dermatoses such an dermatitis, eczema, atopic dermatitis, allergic contact dermatitis, urticaria; vasculitis (e.g., necrotizing, cutaneous, and hypersensitivity vasculitis); eosinophilic myositis, eosinophilic fasciitis; cancers with leukocyte infiltration of the skin or organs, ischemic injury, including cerebral ischemia (e.g., brain injury as a result of trauma, epilepsy, hemorrhage or stroke, each of which may lead to neurodegeneration); HIV, heart failure, chronic, acute or malignant liver disease, autoimmune thyroiditis; systemic lupus erythematosus, Sjorgren's syndrome, lung diseases (e.g., ARDS); acute pancreatitis; amyotrophic lateral sclerosis (ALS); Alzheimer's disease; cachexia/anorexia; asthma; atherosclerosis; chronic fatigue syndrome, fever; diabetes (e.g., insulin diabetes or juvenile onset diabetes); glomerulonephritis; graft versus host rejection (e.g., in transplantation); hemorrhagic shock; hyperalgesia: inflammatory bowel disease; multiple sclerosis; myopathies (e.g., muscle protein metabolism, esp. in sepsis); osteoarthritis; osteoporosis; Parkinson's disease; pain; pre-term labor; psoriasis; reperfusion injury; cytokine-induced toxicity (e.g., septic shock, endotoxic shock); side effects from radiation therapy, temporal mandibular joint disease, tumor metastasis; or an inflammatory condition resulting from strain, sprain, cartilage damage, trauma such as burn, orthopedic surgery, infection or other disease processes.

Allergic diseases and conditions, include but are not limited to respiratory allergic diseases such as asthma, allergic rhinitis, hypersensitivity lung diseases, hypersensitivity pneumonitis, eosinophilic pneumonias (e.g., Loeffler's syndrome, chronic eosinophilic pneumonia), delayed-type hypersensitivity, interstitial lung diseases (ILD) (e.g., idiopathic pulmonary fibrosis, or ILD associated with rheumatoid arthritis, systemic lupus erythematosus, ankylosing spondylitis, systemic sclerosis, Sjogren's syndrome, polymyositis or dermatomyositis); systemic anaphylaxis or hypersensitivity responses, drug allergies (e.g., to penicillin, cephalosporins), insect sting allergies, and the like.

In another aspect, provided is a method of preventing or treating a memory-related disorder in patient in need thereof, comprising administering a therapeutically effective amount of a compound as described herein. Compounds can be used to treat patients with memory impairments associated with direct cognitive disorders such as amnesia, dementia and delirium, frontotemportal dementia; anxiety disorders such as phobias, panic disorders, psychosocial stress (e.g. as seen in disaster, catastrophe or violence victims), obsessive-compulsive disorder, generalized anxiety disorder and post-traumatic stress disorder; mood disorders such as depression and bipolar disorder; and psychotic disorders such as schizophrenia and delusional disorder. Memory impairment, a hallmark of neurodegenerative diseases such as, but not limited to, Parkinson's, Alzheimer's, Huntington's, amyotrophic lateral sclerosis (ALS), spinocerebellar ataxia, as well as aging, can also be treated by using a compound disclosed herein. In addition, compounds disclosed can be used to treat drug addiction through extinction of drug-seeking behavior.

HDAC inhibitors, e.g., HDAC1 and/or HDAC2 selective inhibitors, may also be useful to treat sickle cell disease (SCD) and β-thalassemia (bT). They may also be useful in treating mood disorders or brain disorders with altered chomatin-mediated neuroplasticity (Schoreder, et al., PLoS ONE 8(8): e71323 (2013)).

In another aspect, provided is a method of preventing or treating a hemoglobin disorder in patient in need thereof, comprising administering a therapeutically effective amount of a compound as described herein, or salt thereof. Compounds can be used to treat patients with sickle cell anemia or β-thalassemia. In various embodiments, the compound is a selective HDAC1 and/or HDAC2 inhibitor and is used to prevent or treat the hemoglobin disorder (e.g., sickle cell anemia or β-thalassemia).

Further provided is a method of preventing or treating a mood disorder or brain disorders with altered chromatin-mediated neuroplasticity in patient in need thereof, comprising administering a therapeutically effective amount of a compound as described herein, or salt thereof. Compounds as described herein can be used to treat patients with a mood disorder.

In a further aspect, this application features methods of treating a neurological condition (e.g., Friedreich's ataxia (FRDA), myotonic dystrophy, spinal muscular atrophy, fragile X syndrome, Huntington's disease, a spinocerebellar ataxia, Kennedy's disease, amyotrophic lateral sclerosis, Niemann Pick, Pitt Hopkins, spinal and bulbar muscular atrophy, Alzheimer's disease or schizophrenia, bipolar disorder, and related diseases) that include administering a compound described herein or salt thereof to a patient having a neurological condition.

In another aspect, provided herein is the use of a compound described herein or salt thereof in the preparation of a medicament for the treatment or prevention of a neurological condition (e.g., Friedreich's ataxia, myotonic dystrophy, spinal muscular atrophy, fragile X syndrome, Huntington's disease, a spinocerebellar ataxia, Kennedy's disease, amyotrophic lateral sclerosis, Niemann Pick, Pitt Hopkins, spinal and bulbar muscular atrophy, or Alzheimer's disease); a memory-affecting condition or disease, a cancer; or an inflammatory disorder, or a Plasmodium falciparum infection (e.g., malaria).

Further provided herein is a method of using a compound or a salt thereof as disclosed herein to inhibit class I histone deacetylases, wherein this inhibition results in an in vitro increased frataxin mRNA expression in Friedreich's ataxia patient peripheral blood mononuclear cells (PBMCs). In other embodiments, compounds or a salt thereof disclosed herein inhibit in vitro proliferation of colorectal cancer cells in a dose-dependent fashion. In further embodiments, compounds or a salt thereof disclosed herein increase long term memory in vivo using the novel object recognition paradigm.

In a further aspect, provide herein is a kit for the treatment or prevention of a disorder selected from a neurological disorder (e.g., Friedreich's ataxia, myotonic dystrophy, spinal muscular atrophy, fragile X syndrome, Huntington's disease, a spinocerebellar ataxia, Kennedy's disease, amyotrophic lateral sclerosis, spinal and bulbar muscular atrophy, or Alzheimer's disease), a memory-affecting condition or disease, a cancer, an inflammatory disorder, or a Plasmodium falciparum infection (e.g., malaria) in a patient in need thereof, comprising (i) a compound described herein or a salt thereof; and (ii) instructions comprising a direction to administer said compound to said patient.

In another aspect, provided are methods of treating a neurological condition (e.g., Friedreich's ataxia, myotonic dystrophy, spinal muscular atrophy, fragile X syndrome, Huntington's disease, spinocerebellar ataxias, Kennedy's disease, amyotrophic lateral sclerosis, spinal and bulbar muscular atrophy, or Alzheimer's disease) that include performing any of the above methods, formulating the candidate compound or a salt thereof in a pharmaceutical composition, and administering the pharmaceutical composition to a patient having a neurological condition.

HDAC inhibitors have been shown to have antimalarial activity (Andrews, et al., 2000, Int. J. Parasitol., 30:761-768; Andrews, et al., Antimicrob. Agents Chemother., 52:1454-61). The present disclosure provides methods of treating a Plasmodium falciparum infection (e.g., malaria) in a patient in need thereof.

HDAC inhibitors may also be useful to treat infectious disease such as viral infections. For example, treatment of HIV infected cells with HDAC inhibitors and anti-retroviral drugs can eradicate virus from treated cells (Blazkova, J., et al J Infect Dis. 2012 Sep 1;206(5):765-9; Archin, N. M., et al Nature 2012 Jul. 25, 487(7408):482-5). The present disclosure provides methods of treating a HIV infection in need thereof.

In certain embodiments, provided is a method of treating any of the diseases or disorders described herein comprising administering to a subject in need of treatment thereof a compound or salt thereof according to any of the various embodiments disclosed herein.

### Pharmaceutical Compositions

HDAC inhibitors as disclosed herein can be administered neat or formulated as pharmaceutical compositions. Pharmaceutical compositions include an appropriate amount of the HDAC inhibitor in combination with an appropriate carrier and optionally other useful ingredients. For example, the other useful ingredients include, but not limited to, encapsulating materials or additives such as absorption accelerators, antioxidants, binders, buffers, coating agents, coloring agents, diluents, disintegrating agents, emulsifiers, extenders, fillers, flavoring agents, humectants, lubricants, perfumes, preservatives, propellants, releasing agents, sterilizing agents, sweeteners, solubilizers, wetting agents and mixtures thereof.

In certain embodiments, optionally in combination with any or all of the above various embodiments, provided herein is pharmaceutical composition of a compound disclosed herein, for example a compound of formula (I), a compound of Table 1, or a compound of Table 2, or stereoisomers thereof, or a pharmaceutically acceptable salt thereof, and one or more pharmaceutically acceptable carriers.

In certain embodiments, the pharmaceutical composition comprises a compound of formula (I), or stereoisomers thereof, or a pharmaceutically acceptable salt thereof, and one or more pharmaceutically acceptable carriers. In certain embodiments, pharmaceutical composition comprises a compound of Table 1, or a compound of Table 2, or stereoisomers thereof, or a pharmaceutically acceptable salt thereof, and one or more pharmaceutically acceptable carriers. In certain embodiments, pharmaceutical composition comprises a compound of Table 1, or stereoisomers thereof, or a pharmaceutically acceptable salt thereof, and one or more pharmaceutically acceptable carriers. In certain embodiments, pharmaceutical composition comprises a compound of Table 2, or stereoisomers thereof, or a pharmaceutically acceptable salt thereof, and one or more pharmaceutically acceptable carriers.

Thus, provided herein are pharmaceutical compositions comprising a compound described herein and one or more pharmaceutically acceptable carriers. The pharmaceutical compositions are administered to a subject in need thereof by any route which makes the compound bioavailable. In one embodiment, the composition is a solid formulation adapted for oral administration. In another embodiment, the composition is a tablet, powder, or capsule; or the composition is a tablet. In one embodiment, the composition is a liquid formulation adapted for oral administration. In one embodiment, the composition is a liquid formulation adapted for parenteral administration. In another embodiment, the composition is a solution, suspension, or emulsion; or the composition is a solution. In another embodiment, solid form compositions can be converted, shortly before use, to liquid form compositions for either oral or parenteral administration. These particular solid form compositions are provided in unit dose form and as such are used to provide a single liquid dosage unit. These and other pharmaceutical compositions and processes for preparing the same are well known in the art. (See, for example, Remington: The Science and Practice of Pharmacy (D. B. Troy, Editor, 21st Edition, Lippincott, Williams & Wilkins, 2006).

The dosages may be varied depending on the requirement of the patient, the severity of the condition being treating and the particular compound being employed. Determination of the proper dosage for a particular situation can be determined by one skilled in the medical arts. The total daily dosage may be divided and administered in portions throughout the day or by means providing continuous delivery.

The compounds and compositions described herein may be administered initially in a suitable dosage that may be adjusted as required, depending on the desired clinical response. In certain embodiments, the compounds are administered to a subject at a daily dosage of between 0.01 to about 50 mg/kg of body weight. In other embodiments, the dose is from 1 to 1000 mg/day. In certain embodiments, the daily dose is from 1 to 750 mg/day; or from 10 to 500 mg/day.

In another embodiment, the pharmaceutical composition is in unit dosage form. The composition can be subdivided into unit doses containing appropriate quantities of the active component(s). The unit dosage form can be a tablet, capsule, or powder in a vial or ampule, or it may be the appropriate number of any of these in a packaged form. The unit dosage form can be a packaged form, the package containing discrete quantities of composition such as packeted tablets, capsules, or powders in vials or ampules. The quantity of active compound(s) in a unit dose of the composition may be varied or adjusted from about 1 mg to about 100 mg, or from about 1 mg to about 50 mg, or from about 1 mg to about 25 mg, according to the particular application.

### General Synthesis of Compounds of Formula (I)

Compounds of the present disclosure can be conveniently prepared in accordance with the procedures outlined in the Examples section, from commercially available starting materials, compounds known in the literature, or readily prepared intermediates, by employing conventional synthetic methods and procedures known to those skilled in the art. Conventional synthetic methods and procedures for the preparation of organic molecules and functional group transformations and manipulations can be readily obtained from the relevant scientific literature or from standard textbooks in the field. It will be appreciated that, where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures. Those skilled in the art of organic synthesis will recognize that the nature and order of the synthetic steps presented may be varied for the purpose of optimizing the formation of the compounds described herein.

### EXAMPLES

### Synthesis of Specific Compounds was Performed as Follows

Abbreviations

| Abbreviation | Meaning |
|---|---|
| ACN | acetonitrile |
| AcOH | acetic acid |
| Boc | tert-butyloxycarbonyl |
| Boc₂O | di-tert-butyl dicarbonate |
| Cs₂CO₃ | cesium carbonate |
| DCE | 1,2-dichloroethane |
| DCM | dichloromethane |
| DIPEA | diisoproylethylamine |
| DMAP | 4-dimethylaminopyridine |
| DMF | dimethylformamide |
| DMSO | dimethyl sulfoxide |
| EtOH | ethanol |
| EDCl | 1-ethyl-3-(3-dimethylaminopropyl)carbodiimide |
| Fmoc | fluorenylmethyloxycarbonyl |
| HATU | O-(7-azabenzotriazol-1-yl)-N,N,N′,N′- |
| | tetramethyluronium hexafluorophosphate |
| HCl | hydrochloric acid |
| HPLC | high performance liquid chromatography |
| Hz | Hertz (s⁻¹) |
| HOBt | hydroxybenzotriazole |
| K₂CO₃ | potassium carbonate |
| T₃P | Propylphosphonic acid anhydride |
| LC-MS | liquid chromatography-mass spectrometry |
| mg | milligram |
| MeOH | methanol |
| mL | milliliter |
| mm | millimeter |
| μm | micrometer |
| mmol | millimole |
| μL | microliter |
| mM | millimolar |
| μM | micromolar |
| NMR | Nuclear Magnetic Resonance |
| NaH | sodium hydride |
| STAB | sodiumtriacetoxyborohyride |
| TEA | triethylamine |
| TFA | trifluoroacetic acid |
| THF | tetrahydrofuran |
| TLC | thin layer chromatography |

### Synthetic Scheme for Compound 485 and Compound 486

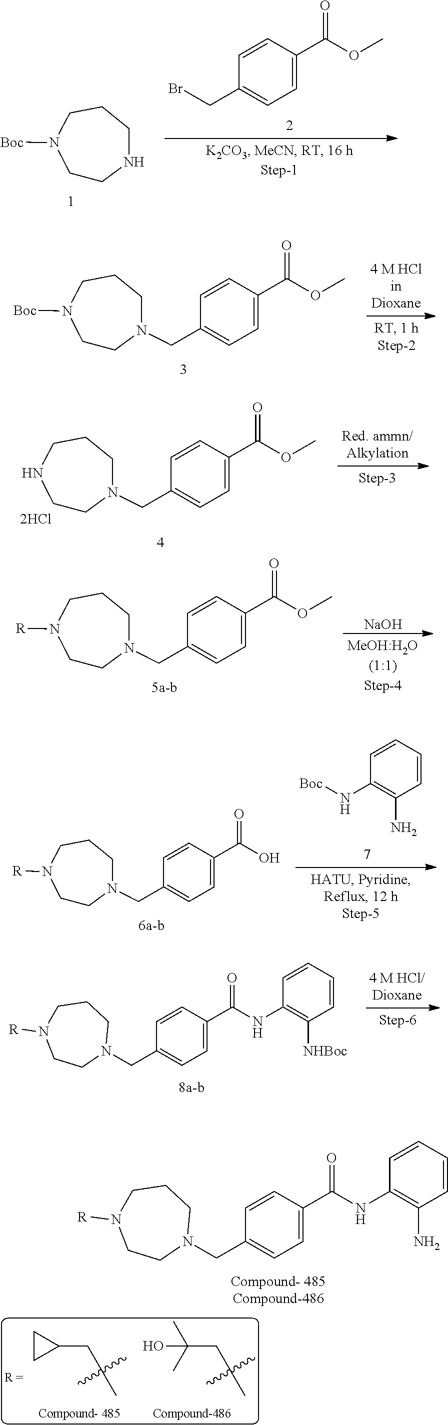

Step 1: Synthesis of tert-butyl 4-(4-(methoxycarbonyl)benzyl)-1,4-diazepane-1-carboxylate (3): To a stirred solution of compound 1 (1.92 g, 1.1 eq.) and compound 2 (2 g, 1 eq.) in ACN (20 mL), potassium carbonate (1.8 g, 1.5 eq.) was added. The reaction mixture was stirred at room temperature for 16 h. The completion of reaction was monitored by TLC. The reaction mixture was diluted with water and extracted with ethyl acetate. The combined organic extracts were washed with water, brine, dried over anhydrous Na₂SO₄, filtered and concentrated under reduced pressure to afford the title compound 3 which was used for next step without further purification.

Step 2: Synthesis of methyl 4-((1,4-diazepan-1-yl)methyl)benzoate hydrochloride (4): To a stirred solution of compound 3 (2.8 g, 1 eq.) in 1,4-dioxane (10 mL), 4 M HCl in dioxane (20 mL) was added at 0° C. The resulting reaction mass was stirred at room temperature for 1 h. After completion of reaction, the reaction mixture was concentrated under reduced pressure, the resulting residue was triturated with diethyl ether and dried under vacuum to afford the title compound 4 as HCl salt.

Step 3: Synthesis of compound 5a for Compound 485: To a stirred solution of compound 4 (1 eq.) and cyclopropyl carboxaldehyde (1.2 eq.) in DCM (10 vol.), acetic acid (6 eq.) was added and stirred at room temperature for 30 min. To this, sodium triacetoxyborohydride (STAB) (3 eq.) was added at room temperature. The resulting reaction mixture was stirred at room temperature for overnight. The reaction mixture was then quenched with sat. NaHCO₃ solution and extracted with DCM. The combined organic extracts were washed with water and brine, dried over anhydrous Na₂SO₄ and evaporated to get the crude product which was purified by silica gel column chromatography to afford the desired compound 5a.

Step 3: Synthesis of compound 5b for Compound 486: To a solution of compound 4 (1 eq) in ethanol (10 vol.), TEA (2.5 eq) and 2,2-dimethyloxirane (1.5 eq) were added. The reaction mixture was heated at 80° C. for 12 h. The completion of reaction was monitored by TLC. The reaction mixture was allowed to cool, concentrated to give the crude product which was purified by silica gel column chromatography to afford the desired compound 5b.

| No | Structure | |
|---|---|---|
| 1 | 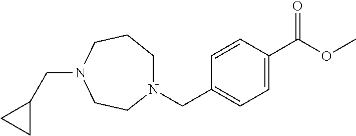 [SMILES: COC(=O)c1ccc(CN2CCCN(CC3CC3)CC2)cc1] | 5a |
| 2 | 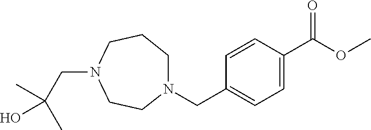 [SMILES: COC(=O)c1ccc(CN2CCCN(CC(C)(C)O)CC2)cc1] | 5b |

Step 4: General procedure for synthesis of compound 6a-b: To a stirred solution of compound 5 (1 eq.) in methanol: water (1:1), NaOH (1.5 eq.) was added at room temperature. The above mixture was heated to 80° C. for 5 to 6 h. The progress of the reaction was monitored by TLC. After completion of reaction, the reaction mixture was concentrated and the resulting residue was dissolved in water and washed with diethyl ether.

The aqueous layer was neutralized to pH=7 using 1 N HCl at 0° C. The solid obtained was filtered, washed with water and dried under vacuum to afford the desired compound 6.

| No | Structure | |
|---|---|---|
| 1 | 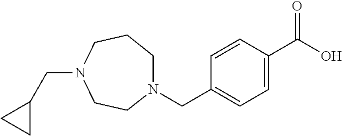 [SMILES: O=C(O)c1ccc(CN2CCCN(CC3CC3)CC2)cc1] | 6a |
| 2 | 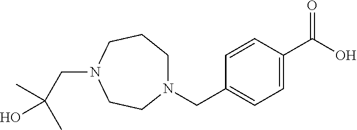 [SMILES: CC(C)(O)CN1CCCN(Cc2ccc(C(=O)O)cc2)CC1] | 6b |

Step 5: General procedure for synthesis of compound 8a-b: To a stirred solution of compound 6 (1 eq.) and tert-butyl (2-aminophenyl)carbamate (1.2 eq.) in ACN, pyridine (6 eq.) and HATU (1.5 eq.) were added at room temperature. The reaction mixture was stirred at 90° C. for overnight and the reaction progress was monitored by TLC and LCMS. After completion of reaction, the reaction mixture was concentrated and resulting residue was portioned between water and ethyl acetate. The organic layer was separated, washed with water and 1% HCl to remove traces of pyridine, dried over anhydrous Na₂SO₄ and concentrated. The crude residue was purified by silica gel column chromatography to afford the desired compound 8.

| No | Structure | |
|---|---|---|
| 1 | 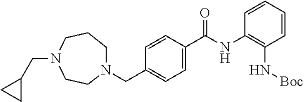 [SMILES: CC(C)(C)OC(=O)Nc1ccccc1NC(=O)c1ccc(CN2CCCN(CC3CC3)CC2)cc1] | 8a |
| 2 | 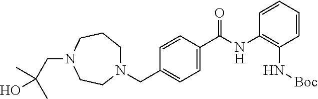 [SMILES: CC(C)(O)CN1CCCN(Cc2ccc(C(=O)Nc3ccccc3NC(=O)OC(C)(C)C)cc2)CC1] | 8b |

Step 6: Synthesis of N-(2-aminophenyl)-4-((4-(cyclopropylmethyl)-1,4-diazepan-1-yl)methyl) benzamide trihydrochloride (Compound 485): To a stirred solution of compound 8 (1 eq.) in 1,4-dioxane (5 vol.), 4 M HCl in dioxane (5 vol.) was added. The reaction mixture was stirred at room temperature for 1 h. The completion of reaction was monitored TLC. The reaction mixture was concentrated, resulting residue was triturated with diethyl ether and dried under vacuum to afford the title compound 485 as a HCl salt.

¹H NMR (400 MHz, DMSO-d6): δ 10.12 (s, 1H), 8.12 (d, J=7.2 Hz, 2H), 7.82 (d, J=8.0 Hz, 2H), 7.33 (d, J=7.2 Hz, 1H), 7.16-7.08 (m, 2H), 6.95-6.93 (m, 1H), 4.46 (s, 2H), 3.67-3.34 (m, 8H), 3.05-3.04 (m, 2H), 2.33-2.26 (m, 2H), 1.12-1.10 (m, 1H), 0.65-0.63 (m, 2H), 0.42-0.40 (m, 2H); LCMS: Calculated for C₂₃H₃₀N₄O for free base: 378.24; Observed: 379.15 (M+1)⁺.

Step 6: Synthesis of N-(2-aminophenyl)-4-((4-(2-hydroxy-2-methylpropyl)-1,4-diazepan-1-yl)methyl) benzamide (Compound 486): To a stirred solution of compound 8 (1 eq.) in 1,4-dioxane (5 vol.), 4 M HCl in dioxane (5 vol.), was added. The resulting reaction mass was stirred at room temperature for 1 h. The completion of reaction was monitored TLC. The reaction mixture was concentrated under reduced pressure. The residue was basified with sat. NaHCO₃ solution and extracted with ethyl acetate. The organic layer was separated, washed with water and brine, dried over anhydrous Na₂SO₄ and concentrated. The crude product was purified by silica gel column chromatography and preparative HPLC to afford the title compound 486.

¹H NMR (400 MHz, DMSO-d6) δ 9.61 (s, 1H), 7.93 (d, J=7.8 Hz, 2H), 7.44 (d, J=7.8 Hz, 2H), 7.16 (d, J=7.8 Hz, 1H), 6.97 (t, J=7.7 Hz, 1H), 6.78 (d, J=7.9 Hz, 1H), 6.59 (t, J=7.5 Hz, 1H), 4.88 (s, 2H), 3.96 (s, 1H), 3.67 (s, 2H), 2.83-2.79 (m, 4H), 2.67-2.56 (m, 4H), 2.38 (s, 2H), 1.70-1.68 (m, 2H), 1.07 (s, 6H); LCMS: Calculated for C₂₃H₃₂N₄O₂: 396.25; Observed: 396.95 (M+1)⁺.

### Synthetic Scheme for Compound 479 and Compound 480

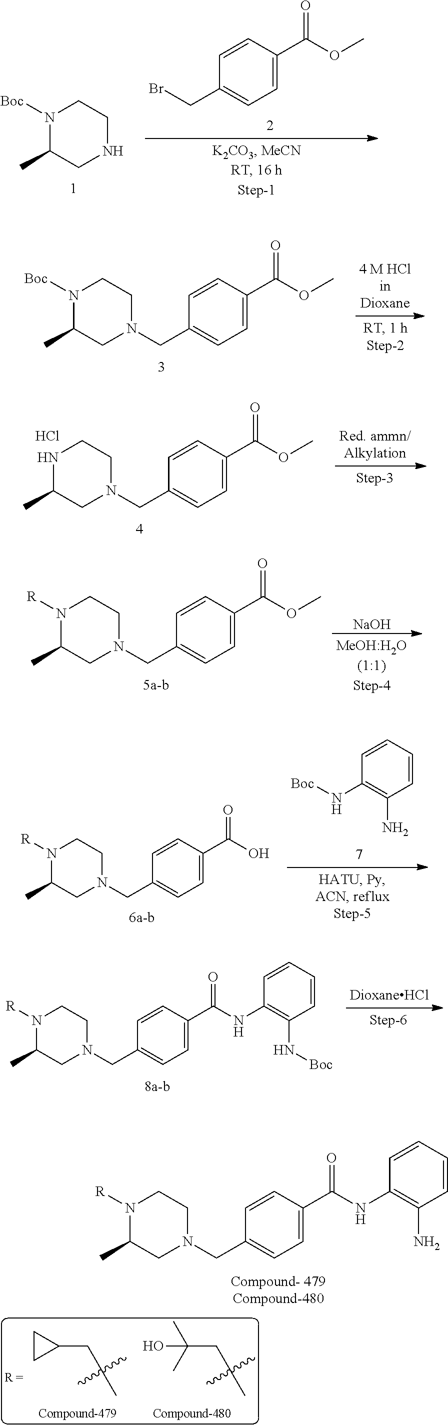

Step 1: Synthesis of tert-butyl (R)-4-(4-(methoxycarbonyl)benzyl)-2-methylpiperazine-1-carboxylate (3): To a stirred solution of compound 1 (1.92 g, 1.1 eq.) and compound 2 (2 g, 1 eq.) in ACN (20 mL), potassium carbonate (1.81 g, 1.5 eq.) was added. The reaction mixture was stirred at room temperature for 16 h. The completion of reaction was monitored by TLC. The reaction mixture was diluted with water and extracted with ethyl acetate. The combined organic extracts were washed with water, brine, dried over anhydrous Na₂SO₄, filtered and concentrated under reduced pressure to get the crude product which was purified by silica gel column chromatography to afford the title compound 3.

Step 2: Synthesis of methyl (R)-4-((3-methylpiperazin-1-yl)methyl)benzoate hydrochloride (4): To a stirred solution of compound 3 (2.9 g, 1 eq.) in 1,4-dioxane (5 mL), 4 M HCl in dioxane (15 mL) was added. The resulting reaction mass was stirred at room temperature for 1 h. The completion of reaction was monitored by TLC. The reaction mixture was concentrated under reduced pressure, resulting residue was triturated with diethyl ether and dried under vacuum to afford the title compound 4 as HCl salt.

Step 3: Synthesis of compound 5a for compound-479: To a stirred solution of compound 4 (1 eq.) and cyclopropyl carboxaldehyde (1.2 eq.) in DCM (10 vol.) was added acetic acid (6 eq.) and sodium triacetoxyborohydride (STAB) (3 eq.) at room temperature. Reaction mixture was stirred at room temperature for overnight. The reaction mixture was then quenched with sat. NaHCO₃ solution and extracted with DCM. The combined organic extracts were washed with water and brine, dried over anhydrous Na₂SO₄ and evaporated to get the crude product which was purified by silica gel column chromatography to afford the desired compound 5a.

Step 3: Synthesis of compound 5b for compound 480: To a solution of compound 4 (1 eq.) in ethanol (10 vol.), TEA (2.5 eq.) and 2,2-dimethyloxirane (1.5 eq.) were added and the reaction mixture was heated at 80° C. for 12 h. The progress of reaction was monitored by TLC. After completion of reaction, the reaction mixture was allowed to cool, concentrated to give a crude compound which was purified by silica gel column chromatography to afford the desired compound 5b.

| No | Structure | |
|---|---|---|
| 1 | 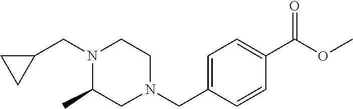 [SMILES: COC(=O)c1ccc(CN2CCN(CC3CC3)[C@H](C)C2)cc1] | 5a |
| 2 | 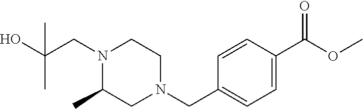 [SMILES: COC(=O)c1ccc(CN2CCN(CC(C)(C)O)[C@H](C)C2)cc1] | 5b |

Step 4: General procedure for synthesis of compound 6a-b: To stirred solution of compound 5 (1 eq.) in methanol:water (1:1) was added NaOH (1.5 eq.) at room temperature. The reaction mixture was heated to 60° C. for 5 to 6 h. The progress of reaction was monitored by TLC. After completion of reaction, the reaction mixture was concentrated and the resulting residue was dissolved in water and washed with diethyl ether. The aqueous layer was neutralized to pH=7 using 1 N HCl at 0° C. The solid obtained was filtered, washed with water and dried under vacuum to provide the desired compound 6.

| No | Structure | |
|---|---|---|
| 1 | 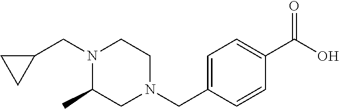 [SMILES: C[C@@H]1CN(Cc2ccc(C(=O)O)cc2)CCN1CC1CC1] | 6a |
| 2 | 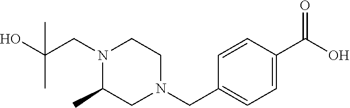 [SMILES: C[C@@H]1CN(Cc2ccc(C(=O)O)cc2)CCN1CC(C)(C)O] | 6b |

Step 5: General procedure for synthesis of compound 8a-b: To a stirred solution of compound 6 (1 eq.) and tert-butyl (2-aminophenyl)carbamate (1.1 eq.) in ACN, pyridine (5 eq.) and HATU (1.5 eq.) were added at room temperature. After stirring the reaction mixture at 80° C. for 12 to 16 h, the reaction progress was monitored by TLC and LCMS. After completion of reaction, the reaction mixture was concentrated and the residue was partitioned between water and ethyl acetate. The organic layer was separated, washed with water and 1% HCl to remove traces of pyridine, dried over anhydrous Na₂SO₄ and concentrated. The crude residue was purified by silica gel column chromatography to afford the desired compound 8.

| No | Structure | |
|---|---|---|
| 1 | 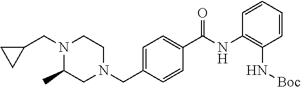 [SMILES: C[C@@H]1CN(Cc2ccc(C(=O)Nc3ccccc3NC(=O)OC(C)(C)C)cc2)CCN1CC1CC1] | 8a |
| 2 | 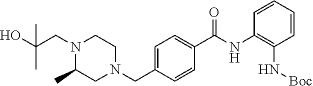 [SMILES: C[C@@H]1CN(Cc2ccc(C(=O)Nc3ccccc3NC(=O)OC(C)(C)C)cc2)CCN1CC(C)(C)O] | 8b |

Step 6: Synthesis of (R)-N-(2-aminophenyl)-4-((4-(cyclopropylmethyl)-3-methylpiperazin-1-yl)methyl)benzamide (Compound 479): To a stirred solution of compound 8 (1 eq) in 1,4-dioxane (5 vol.), 4 M HCl in dioxane (5 vol.) was added. The resulting reaction mass was stirred at room temperature for 1 h. The completion of reaction was monitored TLC. The reaction mixture was concentrated under reduced pressure. The residue was basified with sat. NaHCO₃ solution and extracted with ethyl acetate. The combined organic layers were washed with water and brine, dried over anhydrous Na₂SO₄ and concentrated. The crude residue was purified by silica gel column chromatography and preparative HPLC to afford the desired compound 479.

¹H NMR (400 MHz, DMSO-d6) δ 9.62 (s, 1H), 7.93 (d, J=7.9 Hz, 2H), 7.42 (d, J=7.9 Hz, 2H), 7.19-7.12 (m, 1H), 7.01-6.88 (m, 1H), 6.78-6.76 (m, 1H), 6.64-6.55 (m, 1H), 4.88 (s, 2H), 3.50 (s, 2H), 2.93-2.91 (m, 1H), 2.65-2.53 (m, 3H), 2.17-2.09 (m, 2H), 1.88-1.86 (m, 1H), 0.93 (d, J=6.1 Hz, 3H), 0.81-0.79 (m, 1H), 0.46-0.42 (m, 2H), 0.06-0.05 (m, 2H), 2H merged in solvent peak; LCMS: Calculated for C₂₃H₃₀N₄O: 378.24; Observed: 379.05 (M+1)⁺.

Step 6: Synthesis of (R)-N-(2-aminophenyl)-4-((4-(2-hydroxy-2-methylpropyl)-3-methylpiperazin-1-yl)methyl)benzamide (Compound 480): To a stirred solution of compound 8 (0.3 g, 1 eq.) in 1,4-dioxane (5 vol.), 4 M HCl in dioxane (5 vol.) was added. The resulting reaction mass was stirred at room temperature for 1 h. The completion of reaction was monitored TLC. The reaction mixture was concentrated under reduced pressure. The residue was basified with sat. NaHCO₃ solution and extracted with ethyl acetate. The combined organic layers were washed with water and brine, dried over anhydrous Na₂SO₄ and concentrated. The crude residue was purified by silica gel column chromatography and preparative HPLC to afford title compound 480.

¹H NMR (400 MHz, DMSO-d6) δ 9.62 (s, 1H), 7.93 (d, J=7.9 Hz, 2H), 7.42 (d, J=7.9 Hz, 2H), 7.16 (d, J=7.9 Hz, 1H), 6.97 (t, J=7.6 Hz, 1H), 6.78 (d, J=7.9 Hz, 1H), 6.60 (t, J=7.6 Hz, 1H), 4.88 (s, 2H), 3.96 (s, 1H), 3.49 (s, 2H), 3.10-2.98 (m, 1H), 2.46-2.42 (m, 4H), 2.38-2.33 (m, 2H), 2.26-2.24 (m, 1H), 2.09-1.92 (m, 1H), 1.06 (d, J=4.8 Hz, 6H), 0.94 (d, J=6.0 Hz, 3H). LCMS: Calculated for C₂₃H₃₂N₄O₂: 96.25; Observed: 397.20 (M+1)⁺.

### Synthetic Scheme for Compound 483 and Compound 484

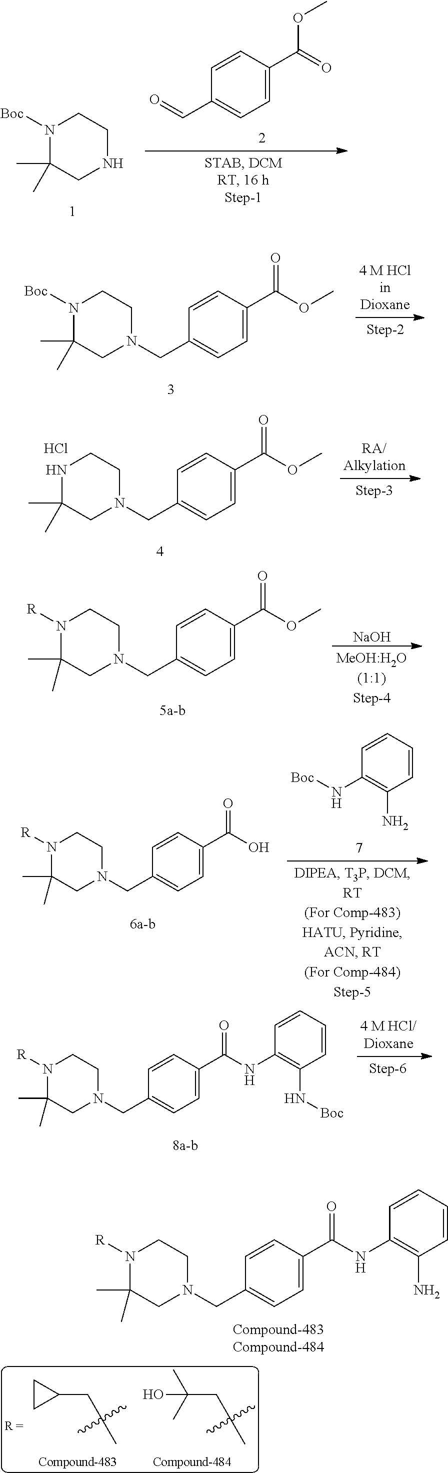

Step 1: Synthesis of tert-butyl 4-(4-(methoxycarbonyl)benzyl)-2,2-dimethylpiperazine-1-carboxylate (3): To a stirred solution of compound 1 (0.4 g, 1 eq.) and aldehyde 2 (0.367 g, 1.2 eq.) in DCM (15 mL), sodium triacetoxyborohydride (STAB) (0.553 g, 1.4 eq.) was added at room temperature. The resulting reaction mixture was stirred at room temperature for 16. The completion of reaction was monitored by TLC and LCMS. The reaction mixture was portioned between DCM and water. The organic layer was washed with water and brine, dried over Na₂SO₄ and evaporated to get the crude product which was purified by silica gel column chromatography to afford the title compound 3.

Step 2: Synthesis of methyl 4-((3,3-dimethylpiperazin-1-yl)methyl)benzoate hydrochloride (4): To a stirred solution of compound 3 (0.5 g, 1 eq.) in 1,4-dioxane (5 mL), 4 M HCl in dioxane (15 mL) was added. The resulting reaction was stirred at room temperature for 1 h. The reaction completion was monitored by TLC. The reaction mixture was concentrated and the resulting residue was triturated with n-pentane and dried under vacuum to afford the title compound 4 as HCl salt.

Step 3: Synthesis of compound 5a for compound 483: To a stirred solution of compound 4 (1 eq.) and cyclopropyl carboxaldehyde (1.2 eq.) in DCM (10 mL), acetic acid (6 eq.) was added and stirred at room temperature for 30 min. To this, sodium triacetoxyborohydride (STAB) (3 eq.) was added at room temperature. The resulting reaction mixture was stirred at room temperature for overnight. The reaction completion was monitored by TLC and LCMS. The reaction mixture was quenched with sat. NaHCO₃ solution and extracted with DCM. The combined organic layers were washed with water and brine, dried over Na₂SO₄ and evaporated to get the crude product which was purified by silica gel column chromatography to afford the desired compound 5a.

Step 3: Synthesis of compound 5b for compound 484: To a solution of compound 4 (1 eq.) in ethanol (10 vol.), TEA (3 eq.) and 2,2-dimethyloxirane (2.6 eq.) were added and the reaction mixture was heated at 80° C. for 12 h. The reaction completion was monitored by TLC. The reaction mixture was allowed to cool, concentrated to give the crude compound which was purified by silica gel column chromatography to afford the desired compound 5.

| No | Structure | |
|---|---|---|
| 1 | 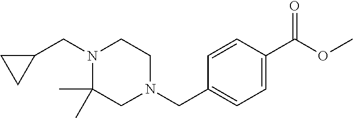 [SMILES: COC(=O)c1ccc(CN2CCN(CC3CC3)C(C)(C)C2)cc1] | 5a |
| 2 | 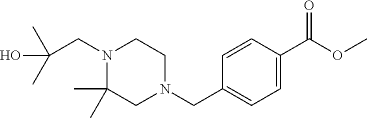 [SMILES: COC(=O)c1ccc(CN2CCN(CC(C)(C)O)C(C)(C)C2)cc1] | 5b |

Step 4: General procedure for synthesis of compound 6a-b: To stirred solution of compound (1.0 eq.) in methanol:water (1:1) was added NaOH (1.5 eq.) at room temperature. The above mixture was heated to 60° C. for 12 to 18 h. The progress of the reaction was monitored by TLC. After completion of reaction, the reaction mixture was concentrated and the resulting residue was dissolved in water and washed with diethyl ether. The aqueous layer was neutralized to pH=7 using 1 N HCl at 0° C. The solid obtained was filtered, washed with water and dried under vacuum to provide the desired compound 6.

| No | Structure | |
|---|---|---|
| 1 | 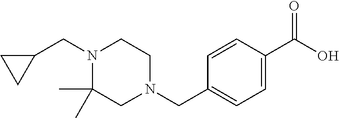 [SMILES: CC1(C)CN(Cc2ccc(C(=O)O)cc2)CCN1CC1CC1] | 6a |
| 2 | 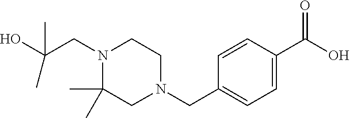 [SMILES: CC(C)(O)CN1CCN(Cc2ccc(C(=O)O)cc2)CC1(C)C] | 6b |

Step 5: Synthesis of compound 8a for compound 483: To a stirred solution of compound 6a (1 eq.) and compound 7 (1.2 eq.) in DCM (10 vol.), DIPEA (2 eq.) and T₃P (1.5 eq.) were added at room temperature. The reaction mixture was stirred at room temperature for 12 h. The reaction completion was monitored by TLC and LCMS. The reaction mixture was portioned between DCM and water. The organic layer was washed with water and brine, dried over Na₂SO₄ and evaporated to get the crude product which was purified by silica gel column chromatography to afford the desired compound 8a.

Step 5: Synthesis of compound 8b for compound 484: To a stirred solution of compound 6b (1 eq.) and compound 7 (1.1 eq.) in ACN (10 vol., pyridine (5 eq.) and HATU (1.5 eq.) were added at room temperature. After stirring the reaction mixture at 80° C. for 12 h, the reaction completion was monitored by TLC and LCMS. The reaction mixture was concentrated and resulting residue was partitioned between water and ethyl acetate. The organic layer was washed with water and 1% HCl to remove traces of pyridine, dried over Na₂SO₄ and concentrated. The crude residue was purified by silica gel column chromatography to afford the desired compound 8b.

| No | Structure | |
|---|---|---|
| 1 | 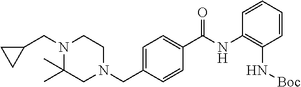 [SMILES: CC(C)(C)OC(=O)Nc1ccccc1NC(=O)c1ccc(CN2CCN(CC3CC3)C(C)(C)C2)cc1] | 8a |
| 2 | 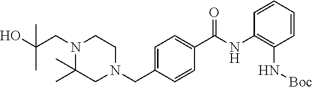 [SMILES: CC(C)(O)CN1CCN(Cc2ccc(C(=O)Nc3ccccc3NC(=O)OC(C)(C)C)cc2)CC1(C)C] | 8b |

Step 6: Synthesis of N-(2-aminophenyl)-4-((4-(cyclopropylmethyl)-3,3-dimethylpiperazin-1-yl)methyl)benzamide (Compound 483): To a stirred solution of compound 8a (1 eq.) in 1,4-dioxane (5vol.), 4 M HCl in dioxane (5 vol.) was added. The resulting reaction mass was stirred at room temperature for 1 h. The reaction completion was monitored TLC. The reaction mixture was concentrated under reduced pressure. The residue was basified with sat. NaHCO₃ solution and extracted with ethyl acetate. The combined organic layers were washed with water and brine, dried over Na₂SO₄ and concentrated. The crude residue was purified by silica gel column chromatography and preparative HPLC to afford the desired compound 483.

¹H NMR (400 MHz, DMSO-d6) δ 9.60 (s, 1H), 7.92 (d, J=7.9 Hz, 2H), 7.41 (d, J=7.9 Hz, 2H), 7.18-7.11 (m, 1H), 7.00-6.91 (m, 1H), 6.76 (d, J=7.9 Hz, 1H), 6.58 (t, J=7.5 Hz, 1H), 4.87 (s, 2H), 3.47 (s, 2H), 2.63-2.61 (m, 2H), 2.41-2.31 (m, 2H), 2.20-2.10 (m, 4H), 0.92 (s, 6H), 0.74-0.72 (m, 1H), 0.41-0.38 (m, 2H), 0.31-0.21 (m, 2H); LCMS: Calculated for C₂₄H₃₂N₄O: 392.26; Observed: 393.20 (M+1)⁺.

Step 6: Synthesis of N-(2-aminophenyl)-4-((4-(2-hydroxy-2-methylpropyl)-3,3-dimethylpiperazin-1-yl)methyl)benzamide (Compound 484): To a stirred solution of compound 8b (0.15 g, 1 eq.) in 1,4-dioxane (5 vol.), 4 M HCl in dioxane (5 vol.) was added. The resulting reaction mass was stirred at room temperature for 1 h. The reaction completion was monitored TLC. The reaction mixture was concentrated under reduced pressure. The residue was basified with sat. NaHCO₃ solution and extracted with ethyl acetate. The combined organic layers were washed with water and brine, dried over Na₂SO₄ and concentrated. The crude residue was purified by silica gel column chromatography and preparative HPLC to afford the desired compound 484.

¹H NMR (400 MHz, DMSO-d6) δ 9.62 (s, 1H), 7.93 (d, J=8.0 Hz, 2H), 7.42 (d, J=7.6 Hz, 2H), 7.16 (d, J=7.6 Hz, 1H), 6.99-6.96 (m, 1H), 6.78-6.76 (m, 1H), 6.61-6.59 (m, 1H), 4.88 (s, 2H), 3.93 (s, 1H), 3.32 (s, 2H), 2.74-2.72 (m, 2H), 2.46-2.32 (m, 2H), 2.10-2.00 (m, 4H), 1.05 (s, 6H), 0.93 (s, 6H); LCMS: Calculated for C₂₄H₃₄N₄O₂: 410.27; Observed: 411.25 (M+1)⁺.

### Synthetic Scheme for Compound 481 and Compound 482

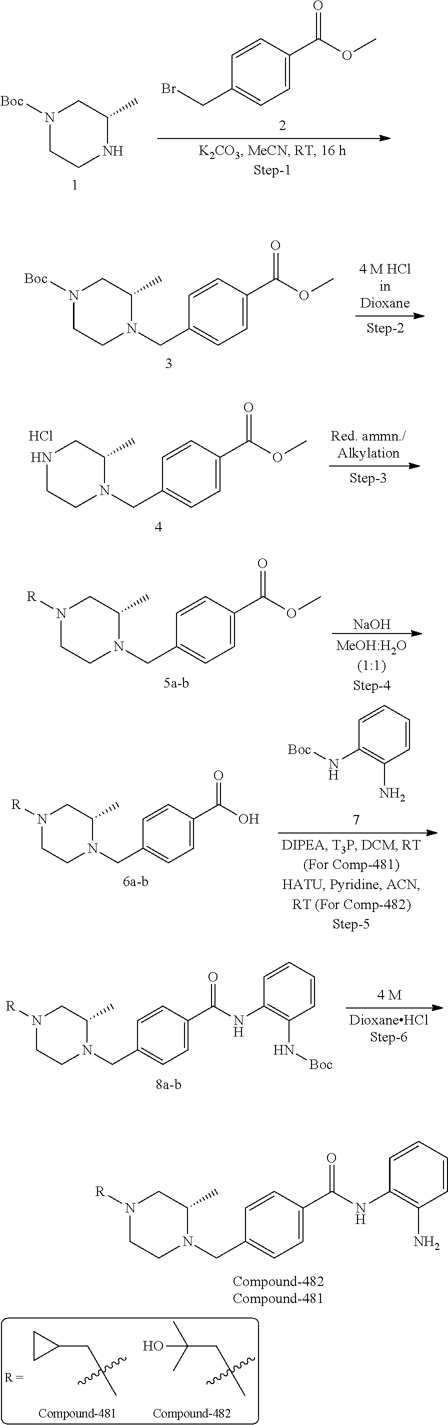

Step 1: Synthesis of tert-butyl (S)-4-(4-(methoxycarbonyl)benzyl)-3-methylpiperazine-1-carboxylate (3): To a stirred solution of compound 1 (2 g, 1 eq.) and compound 2 (2.29 g, 1 eq.) in ACN (20 mL), potassium carbonate (4.2 g, 3 eq.) was added. The reaction mixture was stirred at room temperature for 16 h. The reaction completion was monitored by TLC. The reaction mixture was diluted with water and extracted with ethyl acetate. The combined organic extracts were washed with water, brine, dried over anhydrous Na₂SO₄, filtered and concentrated under reduced pressure to provide a crude residue which was purified by silica gel column chromatography to afford title compound 3.

Step 2: Synthesis of methyl (S)-4-((2-methylpiperazin-1-yl)methyl)benzoate hydrochloride (3): To a stirred solution of compound 3 (2.8 g, 1 eq.) in 1,4-dioxane (15 mL), 4 M HCl in dioxane (5 mL) was added. The reaction mixture was stirred at room temperature for 1 h. The reaction completion was monitored by TLC. The reaction mixture was concentrated and the resulting residue was triturated with n-pentane, diethyl ether and dried under vacuum to afford title compound 4 as HCl salt.

Step 3: Synthesis of compound 5a for Compound 481: To a stirred solution of compound 4 (, 1 eq.) and cyclopropyl carboxaldehyde (1.2 eq.) in DCM (10 vol.), acetic acid (6 eq.) was added and stirred at room temperature for 30 min. To this, sodium triacetoxyborohydride (STAB) (3 eq.) was added at room temperature. The resulting reaction mixture was stirred at room temperature for overnight. The reaction completion was monitored by TLC and LCMS. The reaction mixture was quenched with sat. NaHCO₃ solution and extracted with DCM. The combined organic layers were washed with water and brine, dried over Na₂SO₄ and evaporated to afford the desired compound 5a.

Step 3: Synthesis of compound 5b for Compound 482: To a solution of compound 4 (1 eq.) in ethanol (10 vol.), TEA (3 eq.) and 2,2-dimethyloxirane (1.5 eq.) were added and the reaction mixture was heated at 80° C. for 12 h. The reaction completion was monitored by TLC. The reaction mixture was allowed to cool, concentrated to afford the desired compound 5b.

| No | Structure | |
|---|---|---|
| 1 | 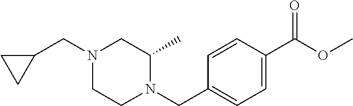 [SMILES: COC(=O)c1ccc(CN2CCN(CC3CC3)C[C@@H]2C)cc1] | 5a |
| 2 | 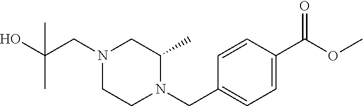 [SMILES: COC(=O)c1ccc(CN2CCN(CC(C)(C)O)C[C@@H]2C)cc1] | 5b |

Step 4: General procedure for synthesis of compound 6a-b: To a stirred solution of compound 5 (1.0 eq.) in methanol:water (1:1) was added NaOH (1.5 eq.) at room temperature. The above mixture was heated to 90° C. for 5 h. The reaction completion was monitored by TLC. The reaction mixture was concentrated and the resulting residue was dissolved in water and washed with diethyl ether. The aqueous layer was neutralized to pH =7 using 1 N HCl at 0° C. The solid obtained was filtered, washed with water and dried under vacuum to afford the desired compound 6.

| No | Structure |
|---|---|
| 1 | 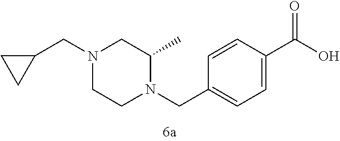 [SMILES: C[C@H]1CN(CC2CC2)CCN1Cc1ccc(C(=O)O)cc1] |
| 2 | 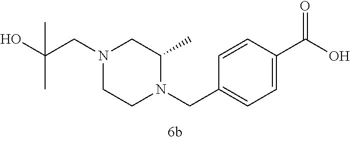 [SMILES: C[C@H]1CN(CC(C)(C)O)CCN1Cc1ccc(C(=O)O)cc1] |

Step 5: Synthesis of compound 8a for Compound 481: To a stirred solution of compound 6a (1 eq.) and compound 7 (1 eq.) in DCM (10 vol.), DIPEA (2 eq.) T₃P (1.5 eq.) was added at room temperature. After stirring the reaction mixture at ambient temperature for overnight, the reaction mixture was portioned between DCM and water. The combined organic extracts were washed with water and brine, dried over Na₂SO₄ and evaporated to get the crude product which was purified by silica gel column chromatography to afford the desired compound 8a.

Step 5: Synthesis of compound 8b for Compound 482: To a stirred solution of compound 6b (1 eq) and compound 7 (1.1 eq.) in ACN (10 vol.), pyridine (5 eq.) and HATU (1.5 eq.) were added at room temperature. After stirring the reaction mixture at 80° C. for overnight, the reaction mixture was cooled, concentrated and resulting residue was partitioned between water and ethyl acetate. The combined organic extracts were washed with water and 1% HCl to remove traces of pyridine, dried over Na₂SO₄ and concentrated. The crude residue was purified by silica gel column chromatography to afford desired compound 8b.

| No | Structure |
|---|---|
| 1 | 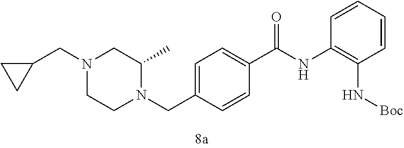 [SMILES: C[C@H]1CN(CC2CC2)CCN1Cc1ccc(C(=O)Nc2ccccc2NC(=O)OC(C)(C)C)cc1] |
| 2 | 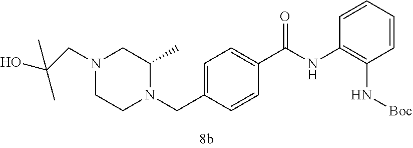 [SMILES: C[C@H]1CN(CC(C)(C)O)CCN1Cc1ccc(C(=O)Nc2ccccc2NC(=O)OC(C)(C)C)cc1] |

Step 6: Synthesis of (S)-N-(2-aminophenyl)-4-((4-(2-hydroxy-2-methylpropyl)-2-methylpiperazin-1-yl)methyl)benzamide (Compound 482): To a stirred solution of compound 8b (1 eq.) in 1,4-dioxane (5 vol.), 4 M HCl in dioxane (5vol.) was added. The resulting reaction mass was stirred at room temperature for 1 h. The reaction completion was monitored TLC. The reaction mixture was concentrated under reduced pressure. The crude residue was purified by preparative HPLC to afford the desired compound 482.

¹H NMR (400 MHz, DMSO-d6) δ 9.60 (s, 1H), 7.91 (d, J=7.6 Hz, 2H), 7.40 (d, J=8.0 Hz, 2H), 7.18-7.11 (m, 1H), 6.97-6.93 (m, 1H), 6.76 (d, J=8.0 Hz, 1H), 6.60-6.56 (m, 1H), 4.87 (s, 2H), 4.03-3.92 (m, 2H), 3.24-3.22 (m, 1H), 2.75-2.73 (m, 2H), 2.66-2.63 (m, 1H), 2.44-2.42 (m, 1H), 2.29-2.19 (m, 1H), 2.19-2.04 (m, 4H), 1.08-1.02 (m, 9H); LCMS: Calculated for C₂₃H₃₂N₄O₂: 396.25; Observed: 397 (M+1)⁺.

Step 6: Synthesis of (S)-N-(2-aminophenyl)-4-((4-(cyclopropylmethyl)-2-methylpiperazin-1-yl)methyl)benzamide (Compound-481): To a stirred solution of compound 8a (0.15 g, 1 eq) in 1,4-dioxane (5 vol.), 4 M HCl in dioxane (5 vol.) was added. The resulting reaction mass was stirred at room temperature for 1 h. The reaction completion was monitored TLC. The reaction mixture was concentrated under reduced pressure. The residue was basified with sat. NaHCO₃ solution and extracted with ethyl acetate. The combined organic extracts were washed with water and brine, dried over Na₂SO₄ and concentrated. The crude residue was purified by silica gel column chromatography and preparative HPLC to afford the desired compound 481.

¹H NMR (400 MHz, DMSO-d6) δ 9.61 (s, 1H), 7.93 (d, J=8.0 Hz, 2H), 7.42 (d, J=7.6 Hz, 2H), 7.19-7.12 (m, 1H), 7.01-6.92 (m, 1H), 6.78 (d, J=6.8 Hz, 1H), 6.60 (t, J=7.6 Hz, 1H), 4.88 (s, 2H), 4.04-4.00 (m, 1H), 3.22-3.18 (m, 1H), 2.79-2.64 (m, 2H), 2.61-2.53 (m, 1H), 2.43-2.41 (m, 1H), 2.19-2.05 (m, 4H), 2.00-1.92 (m, 1H), 1.08 (d, J=6.4 Hz, 3H), 0.80-0.78 (m, 1H), 0.44-0.41 (m, 2H), 0.06-0.02 (m, 2H); LCMS: Calculated for C₂₃H₃₀N₄O: 378.24; Observed: 379.20 (M+1)⁺.

### Synthetic Scheme for Compound 489 and Compound 490

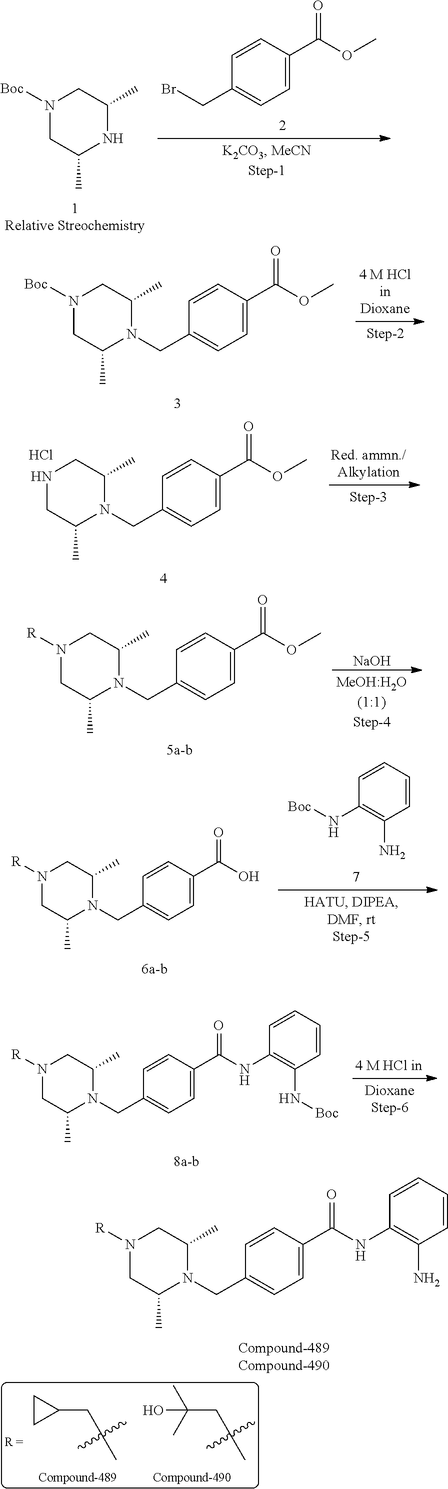

Step 1: Synthesis of tert-butyl (3R,5S)-4-(4-(methoxycarbonyl)benzyl)-3,5-dimethylpiperazine-1-carboxylate (3): To a stirred solution of compound 1 (2.1 g, 1 eq.) and compound 2 (2.3 g, 1 eq.) in ACN (20 mL), potassium carbonate (4.1 g, 3 eq.) was added. The reaction mixture was stirred at room temperature for 16 h. The reaction completion was monitored by TLC. The reaction mixture was diluted with water and extracted with ethyl acetate. The combined organic extracts were washed with water and brine, dried over anhydrous Na₂SO₄, filtered and concentrated under reduced pressure to give a crude residue which was purified by silica gel column chromatography to afford compound 3.

Step 2: Synthesis of methyl 4-(((2R,6S)-2,6-dimethylpiperazin-1-yl)methyl)benzoate hydrochloride (4): To a stirred solution of compound 3 (2.5 g, 1 eq.) in 1,4-dioxane (5 mL), 4 M HCl in dioxane (3 mL) was added. The reaction mixture was stirred at room temperature for 1 h. The reaction completion was monitored by TLC. The reaction mixture was concentrated and the resulting residue was triturated with n-pentane and dried under vacuum to afford the desired compound 4 as HCl salt.

Step 3: Synthesis of compound 5a for Compound-489: To a stirred solution of compound 4 (1 eq.) and cyclopropyl carboxaldehyde (1.2 eq.) in DCM (10 vol.), acetic acid (6 eq.) was added and stirred at room temperature for 30 min. To this, sodium triacetoxyborohydride (STAB) (3 eq.) was added at room temperature. The resulting reaction mixture was stirred at room temperature for overnight. The reaction completion was monitored by TLC and LCMS. The reaction mixture was quenched with sat. NaHCO₃ solution and extracted with DCM. The combined organic extracts were washed with water and brine, dried over Na₂SO₄ and evaporated to afford the desired compound 5a.

Step 3: Synthesis of compound 5b for Compound 490: To a solution of compound 4 (1 eq.) in ethanol (10 vol.), TEA (3 eq.) and 2,2-dimethyloxirane (1.5 eq.) were added and the reaction mixture was heated at 80° C. for 12 h. The reaction completion was monitored by TLC. The reaction mixture was allowed to cool and concentrated to afford the desired compound 5b.

| No | Structure |
|---|---|
| 1 | 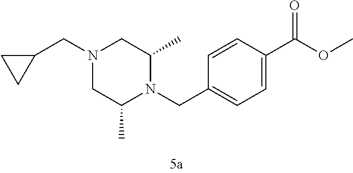 [SMILES: COC(=O)c1ccc(CN2[C@H](C)CN(CC3CC3)C[C@@H]2C)cc1] |
| 2 | 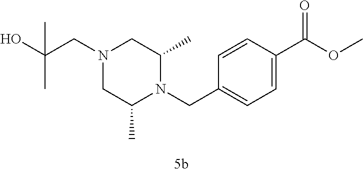 [SMILES: COC(=O)c1ccc(CN2[C@H](C)CN(CC(C)(C)O)C[C@@H]2C)cc1] |

Step 4: General procedure for synthesis of compound 6a-b: To a stirred solution of compound 5 (1.0 eq.) in methanol:water (1:1) was added NaOH (1.5 eq.) at room temperature. The reaction mixture was heated to 90° C. for 5 h. The reaction completion was monitored by TLC. The reaction mixture was concentrated and the resulting residue was dissolved in water and washed with diethyl ether. The aqueous layer was neutralized to pH=7 using 1 N HCl at 0° C. The solid obtained was filtered, washed with water and dried under vacuum to afford the desired compound 6.

| No | Structure |
|---|---|
| 1 | 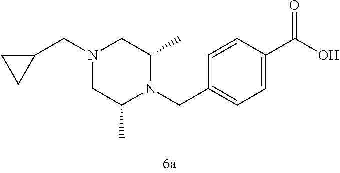 [SMILES: C[C@@H]1CN(CC2CC2)C[C@H](C)N1Cc1ccc(C(=O)O)cc1] |
| 2 | 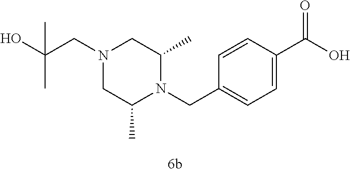 [SMILES: C[C@@H]1CN(CC(C)(C)O)C[C@H](C)N1Cc1ccc(C(=O)O)cc1] |

Step 5: General procedure for synthesis of compound 8a-b: To a stirred solution of compound 6 (1 eq.) and compound 7 (1.2 eq.) in DMF (5 mL), DIPEA (3 eq.) was added and stirred for 10 min. To this, HATU (1.5 eq.) was added and the reaction mixture was stirred at room temperature for overnight. The reaction progress was monitored by TLC and LCMS. After completion of reaction, the reaction mixture was partitioned between ethyl acetate and water. The organic layer was washed with water and brine, dried over anhydrous Na₂SO₄, filtered and evaporated to get the crude product which was purified by silica gel column chromatography to afford the desired compound 8.

| No | Structure |
|---|---|
| 1 | 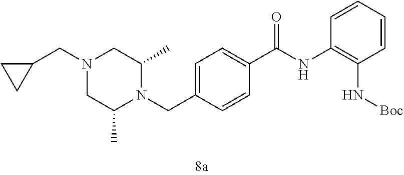 [SMILES: C[C@@H]1CN(CC2CC2)C[C@H](C)N1Cc1ccc(C(=O)Nc2ccccc2NC(=O)OC(C)(C)C)cc1] |
| 2 | 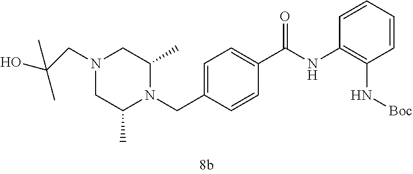 [SMILES: C[C@@H]1CN(CC(C)(C)O)C[C@H](C)N1Cc1ccc(C(=O)Nc2ccccc2NC(=O)OC(C)(C)C)cc1] |

Step 6: Synthesis of N-(2-aminophenyl)-4-(((2R,6S)-4-(cyclopropylmethyl)-2,6-dimethylpiperazin-1-yl)methyl)benzamide (Compound 489): To a stirred solution of compound 8a (1 eq.) in 1,4-dioxane (5 vol.), 4 M HCl in dioxane (5 vol.) was added. The resulting reaction mass was stirred at room temperature for 1 h. The reaction completion was monitored TLC. The reaction mixture was concentrated under reduced pressure. The residue was basified with sat. NaHCO₃ solution and extracted with ethyl acetate. The combined organic extracts were washed with water and brine, dried over Na₂SO₄, filtered and concentrated. The crude residue was purified by silica gel column chromatography and preparative HPLC to afford the title compound 489.

¹H NMR (400 MHz, DMSO-d6) δ 9.58 (s, 1H), 7.90 (d, J=7.9 Hz, 2H), 7.48 (d, J=7.9 Hz, 2H), 7.16 (d, J=7.2 Hz, 1H), 7.01-6.92 (m, 1H), 6.77 (d, J=6.8 Hz, 1H), 6.61-6.59 (m, 1H), 4.88 (s, 2H), 3.78 (s, 2H), 2.84-2.81 (m, 2H), 2.60-2.56 (m, 2H), 2.14-2.05 (m, 2H), 1.77 (t, J=10.6 Hz, 2H), 0.92 (d, J=6.0 Hz, 6H), 0.87-0.73 (m, 1H), 0.49-0.38 (m, 2H), 0.10-0.03 (m, 2H); LCMS: Calculated for C₂₄H₃₂N₄O: 392.26; Observed: 393.20 (M+1)⁺.

Step 6: Synthesis of N-(2-aminophenyl)-4-(((2R,6S)-4-(2-hydroxy-2-methylpropyl)-2,6-dimethylpiperazin-1-yl)methyl)benzamide (Compound 490): To a stirred solution of compound 8b (1 eq.) in 1,4-dioxane (5 vol.), 4 M HCl in dioxane (5 vol.) was added. The resulting reaction mass was stirred at room temperature for 1 h. The reaction completion was monitored TLC. The reaction mixture was concentrated under reduced pressure. The residue was basified with sat. NaHCO₃ solution and extracted with ethyl acetate. The combined organic extracts were washed with water and brine, dried over Na₂SO₄, filtered and concentrated. The crude residue was purified by silica gel column chromatography and preparative HPLC to afford the title compound 490.

¹H NMR (400 MHz, DMSO-d6) δ 9.58 (s, 1H), 7.90 (d, J=7.9 Hz, 2H), 7.48 (d, J=7.9 Hz, 2H), 7.15 (d, J=8.0 Hz, 1H), 6.98-6.94 (m, 1H), 6.77 (d, J=7.2 Hz, 1H), 6.64-6.55 (m, 1H), 4.88 (s, 2H), 4.03 (s, 1H), 3.77 (s, 2H), 2.86-2.78 (m, 2H), 2.60-2.57 (m, 2H), 2.13 (s, 2H), 1.96 (t, J=10.6 Hz, 2H), 1.07 (s, 6H), 0.89 (d, J=6.0 Hz, 6H); LCMS: Calculated for C₂₄H₃₄N₄O₂: 410.27; Observed: 411.25 (M+1)⁺.

### Synthetic Scheme for Compound 491 and Compound 492

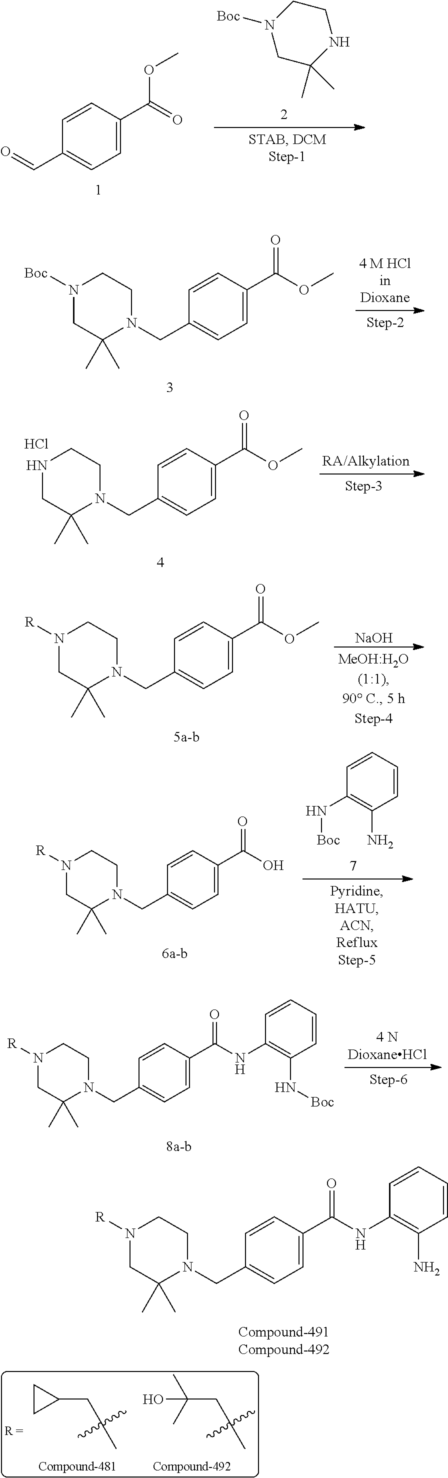

Step 1: Synthesis of tert-butyl 4-(4-(methoxycarbonyl)benzyl)-3,3-dimethylpiperazine-1-carboxylate (3): To a stirred solution of amine compound 2 (0.5 g, 1 eq) and aldehyde 1 (0.421 g, 1.1 eq) in DCM (10 mL), sodium triacetoxyborohydride (STAB) (0.693 g, 1.4 eq) was added. The reaction mixture was stirred at room temperature for overnight; the reaction progress was monitored by TLC and LCMS. After completion of reaction, the reaction mixture was partitioned between DCM and water. The organic layers were separated, washed with water and brine, dried over Na₂SO₄ and evaporated to get the crude product which was purified by silica gel column chromatography to afford the desired compound 3.

Step 2: Synthesis of methyl 4-((2,2-dimethylpiperazin-1-yl)methyl)benzoate hydrochloride (4): To a stirred solution of Boc compound 3 (0.6 g, 1 eq) in 1,4-dioxane (5 mL), 4 M HCl in dioxane (5 mL) was added. The reaction mixture was stirred at room temperature for 1 h. The reaction progress was monitored by TLC. After completion, the reaction mixture was concentrated and the resulting residue was triturated with n-pentane and dried under vacuum to give the desired compound 4 as HCl salt.

Step 3: Synthesis of compound 5a: To a stirred solution of amine compound 4 (1 eq) and corresponding aldehyde (1.2 eq) in DCM (10 vol.), acetic acid (6 eq) was added and stirred at room temperature for 30 min. To this, sodium triacetoxyborohydride (STAB) (3 eq) was added at room temperature. The resulting reaction mixture was stirred at room temperature for overnight; the reaction progress was monitored by TLC and LCMS. After completion, the reaction mixture was quenched with sat. NaHCO₃ solution and extracted with DCM. The organic layers were separated, washed with water and brine, dried over Na₂SO₄ and evaporated to get the crude product which was purified by silica gel column chromatography to afford the desired compound 5a.

Step 3: Synthesis of compound 5b: To a solution of compound 4 (0.4 g, 1 eq) in ethanol (5 vol.), TEA (2.5 eq) and 2,2-dimethyloxirane (1.5 eq) were added and the reaction mixture was heated at 80° C. for 12 h. The progress of reaction was monitored by TLC. After completion, the reaction mixture was allowed to cool, concentrated to give a crude compound which was purified by silica gel column chromatography to afford the desired compound 5b.

| No | Structure |
|---|---|
| 1 | 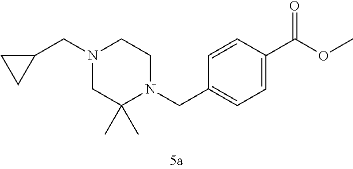 [SMILES: COC(=O)c1ccc(CN2CCN(CC3CC3)CC2(C)C)cc1] |
| 2 | 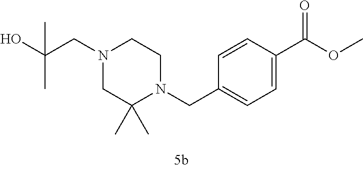 [SMILES: COC(=O)c1ccc(CN2CCN(CC(C)(C)O)CC2(C)C)cc1] |

Step 4: Synthesis of compound 6a-b: To stirred solution of ester compound in Methanol: Water (1:1) was added NaOH (1.5 eq) at room temperature. The above mixture was heated to 90° C. for 5 h. The progress of the reaction was monitored by TLC. After completion of reaction, the reaction mixture was concentrated and the resulting residue was portioned between diethyl ether and water. The aqueous layer was neutralized to pH=7 using 1 N HCl at 0° C. The solid obtained was filtered, washed with water and dried under vacuum to provide the desired compound.

| No | Structure |
|---|---|
| 1 | 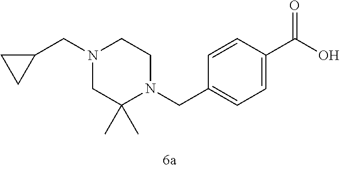 [SMILES: CC1(C)CN(CC2CC2)CCN1Cc1ccc(C(=O)O)cc1] |
| 2 | 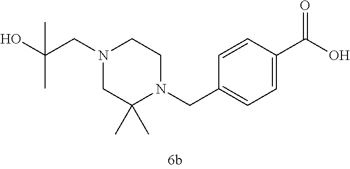 [SMILES: CC(C)(O)CN1CCN(Cc2ccc(C(=O)O)cc2)C(C)(C)C1] |

Step 5: Synthesis of compound 8 a-b: To a stirred solution of acid compound 6 (1 eq) and amine (1.1 eq) in ACN, pyridine (6 eq) and HATU (1.5 eq) were added at room temperature. After stirring the reaction mixture at 80° C. for 12 h, the reaction progress was monitored by TLC and LCMS. After completion, the reaction mixture was concentrated and resulting residue was partitioned between water and ethyl acetate. The organic layers were separated, washed with water and 1% HCl to remove traces of pyridine, dried over Na₂SO₄ and concentrated. The crude residue was purified by silica gel column chromatography to provide the desired compound.

| No | Structure |
|---|---|
| 1 | 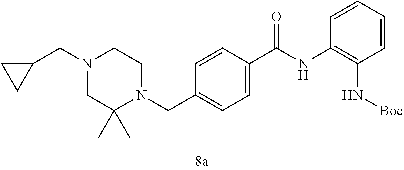 [SMILES: CC(C)(C)OC(=O)Nc1ccccc1NC(=O)c1ccc(CN2CCN(CC3CC3)CC2(C)C)cc1] |
| 2 | 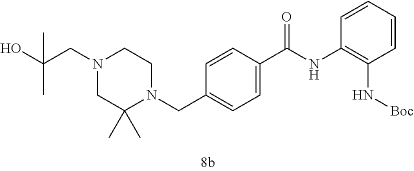 [SMILES: CC(C)(O)CN1CCN(Cc2ccc(C(=O)Nc3ccccc3NC(=O)OC(C)(C)C)cc2)C(C)(C)C1] |

Step 6: Synthesis of N-(2-aminophenyl)-4-((4-(2-hydroxy-2-methylpropyl)-2,2-dimethylpiperazin-1-yl)methyl)benzamide (Compound 492): To a stirred solution of Boc compound 8b (1 eq) in 1,4-dioxane (5 vol.), 4 M HCl in dioxane (5 vol.) was added. The resulting reaction mass was stirred at room temperature for 1 h. The progress of the reaction was monitored TLC. After completion, the reaction mixture was concentrated under reduced pressure. The residue was basified with sat. NaHCO₃ solution and extracted with ethyl acetate. The organic layers were separated, washed with water and brine, dried over Na₂SO₄ and concentrated. The crude residue was purified by silica gel column chromatography/prep. HPLC to provide the desired compound.

¹H NMR (400 MHz, DMSO-d6) δ 9.59 (s, 1H), 7.91 (d, J=8.4 Hz, 2H), 7.43 (d, J=8.4 Hz, 2H), 7.16 (d, J=7.6 Hz, 1H), 6.99-6.94 (m, 1H), 6.79-6.77 (m, 1H), 6.62-6.57 (m, 1H), 4.88 (s, 2H), 4.02 (s, 1H), 3.60-3.51 (m, 2H), 2.42-2.33 (m, 6H), 2.12 (s, 2H), 1.11-1.08 (m, 12H); LCMS Calculated for C₂₄H₃₄N₄O₂: 410.27; Observed: 411.30 (M+1)⁺.

Step 6: Synthesis of N-(2-aminophenyl)-4-((4-(cyclopropylmethyl)-2,2-dimethylpiperazin-1-yl)methyl)benzamide (Compound 491): To a stirred solution of Boc compound 8a (0.14 g, 1 eq) in 1,4-dioxane (5 vol.), 4 M HCl in dioxane (5 vol.) was added. The resulting reaction mass was stirred at room temperature for 1 h. The progress of the reaction was monitored TLC. After completion, the reaction mixture was concentrated under reduced pressure. The residue was basified with sat. NaHCO₃ solution and extracted with ethyl acetate. The organic layers were separated, washed with water and brine, dried over Na₂SO₄ and concentrated. The crude residue was purified by silica gel column chromatography/prep. HPLC to provide the desired compound.

¹H NMR (400 MHz, DMSO-d6) δ 9.59 (s, 1H), 7.91 (d, J=8.4 Hz, 2H), 7.43 (d, J=8.4 Hz, 2H), 7.16 (d, J=7.6 Hz, 1H), 6.98-6.94 (m, 1H), 6.79-6.77 (m, 1H), 6.61-6.59 (m, 1H), 4.87 (s, 2H), 3.60-3.52 (m, 2H), 2.34-2.25 (m, 6H), 2.12-2.10 (m, 2H), 1.12 (s, 6H), 0.88-0.75 (m, 1H), 0.49-0.39 (m, 2H), 0.09-0.03 (m, 2H); LCMS Calculated for C₂₄H₃₂N₄O: 392.26; Observed: 393.30 (M+1)⁺.

### Synthetic Scheme for Compound 487 and Compound 488

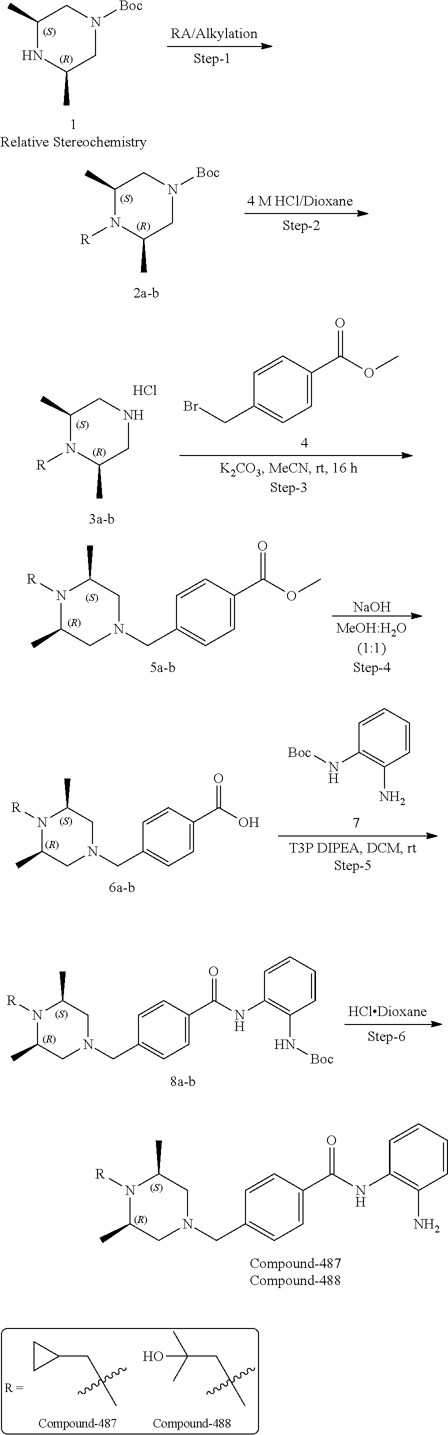

Step 1: Synthesis of compound 2a: To a stirred solution of amine compound 1 (1 eq) and cyclopropyl carboxaldehyde (1.2 eq) in DCM (10 vol.), acetic acid (6 eq) was added and stirred at room temperature for 30 min. To this, sodium triacetoxyborohydride (STAB) (3 eq) was added at room temperature. The resulting reaction mixture was stirred at room temperature for overnight; the reaction progress was monitored by TLC and LCMS. After completion, the reaction mixture was quenched with sat. NaHCO₃ solution and extracted with DCM. The organic layers were separated, washed with water and brine, dried over Na₂SO₄ and evaporated to get the crude product which was purified by silica gel column chromatography to afford the desired compound 2a.

Step 1: Synthesis of compound 2b: To a solution of compound 1 (1 eq) in ethanol (10 mL), TEA (3 eq) and 2,2-dimethyloxirane (1.5 eq) were added and the reaction mixture was heated at 80° C. for 12 h. The progress of reaction was monitored by TLC. After completion, the reaction mixture was allowed to cool, concentrated to give a crude compound which was purified by silica gel column chromatography to afford the desired compound 2b.

| No | Structure |
|---|---|
| 1 | 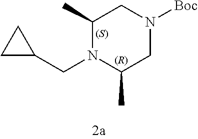 |
| 2 | 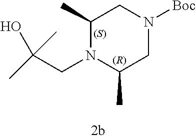 |

Step 2: Synthesis of compounds 3 a-b: To a stirred solution of Boc compound 3 (1 eq) in 1,4-dioxane (5 vol.), 4 M HCl in dioxane(5 vol.) was added. The reaction mixture was stirred at room temperature for 1 h. The reaction progress was monitored by TLC. After completion, the reaction mixture was concentrated and the resulting residue was triturated with n-pentane and dried under vacuum to give the desired compound 3 as HCl salt.

| No | Structure |
|---|---|
| 1 | 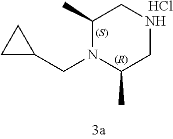 |
| 2 | 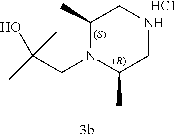 |

Step 3: Synthesis of compound 5a-b: To a stirred solution of compound 3 (1 eq) and compound 4 (1 eq) in ACN, potassium carbonate (3 eq) was added. The reaction mixture was stirred at room temperature for 16 h. The progress of reaction was monitored by TLC. After completion, the reaction mixture was diluted with water and extracted with ethyl acetate. The combined organic extracts were washed with water, brine, dried over anhydrous Na₂SO₄, filtered and concentrated under reduced pressure to provide a crude residue which was purified by silica gel column chromatography to afford compound 5.

| No | Structure |
|---|---|
| 1 | 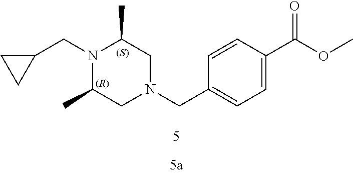 |
| 2 | 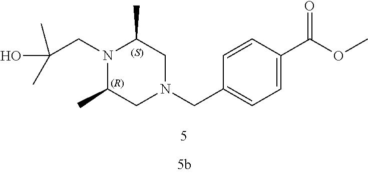 |

Step 4: Synthesis of compound 6a-b: To stirred solution of ester compound in Methanol: Water (1:1) was added NaOH (1.5 eq) at room temperature. The above mixture was heated to 90° C. for 5 h. The progress of the reaction was monitored by TLC. After completion of reaction, the reaction mixture was concentrated and the resulting residue was partitioned between diethyl ether and water. The aqueous layer was neutralized to pH=7 using 1 N HCl at 0° C. The solid obtained was filtered, washed with water and dried under vacuum to provide the desired compound.

| No | Structure |
|---|---|
| 1 | 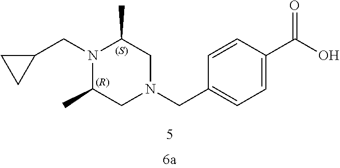 |
| 2 | 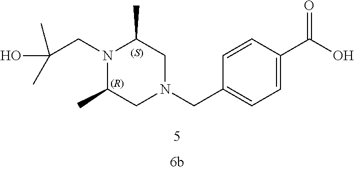 |

Step 5: Synthesis of compound 8a-b: To a stirred solution of compound 6 (1 eq) and compound 7 (1.2 eq) in DCM, DIPEA (2 eq) and T₃P (1.5 eq) were added at room temperature. The reaction mixture was stirred at room temperature for 12 h. The reaction progress was monitored by TLC and LCMS. After completion of reaction, the reaction mixture was portioned between DCM and water. The organic layers were separated, washed with water and brine, dried over Na₂SO₄ and evaporated to get the crude product which was purified by silica gel column chromatography to afford the desired compound 8.

| No | Structure |
|---|---|
| 1 | 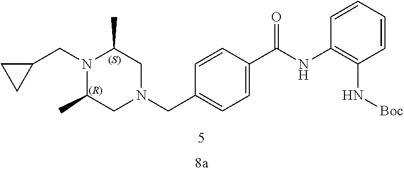 |
| 2 | 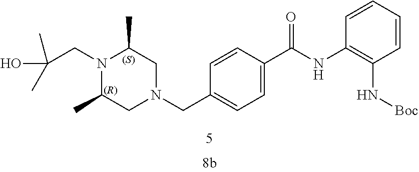 |

Step 6: Synthesis of N-(2-aminophenyl)-4-(((3R,5S)-4-(cyclopropylmethyl)-3,5-dimethylpiperazin-1-yl)methyl)benzamide (Compound 487): To a stirred solution of Boc compound 8a (0.18 g, 1 eq) in 1,4-dioxane (5 vol.), 4 M HCl in dioxane (5 vol.) was added. The resulting reaction mass was stirred at room temperature for 1 h. The progress of the reaction was monitored TLC. After completion, the reaction mixture was concentrated under reduced pressure. The residue was basified with sat. NaHCO₃ solution and extracted with ethyl acetate. The organic layers were separated, washed with water and brine, dried over Na₂SO₄ and concentrated. The crude residue was purified by silica gel column chromatography/prep. HPLC to provide the desired compound.

¹H NMR (400 MHz, DMSO-d6) δ 9.62 (s, 1H), 7.94 (d, J=8.0 Hz, 2H), 7.42 (d, J=8.4 Hz, 2H), 7.16 (d, J=8.0 Hz, 1H), 7.01-6.92 (m, 1H), 6.78 (d, J=8.0 Hz, 1H), 6.64-6.55 (m, 1H), 4.89 (s, 2H), 3.46 (s, 2H), 2.80-2.78 (m, 2H), 2.66-2.64 (m, 2H), 2.59-2.57 (m, 2H), 1.74 (t, J=10.4 Hz, 2H), 0.95 (d, J=6.0 Hz, 6H), 0.85-0.83 (m, 1H), 0.43-0.39 (m, 2H), 0.07-0.06 (m, 2H); LCMS Calculated for C₂₄H₃₂N₄O: 392.26; Observed: 393.20 (M+1)⁺.

Step 6: Synthesis of N-(2-aminophenyl)-4-(((3R,5S)-4-(2-hydroxy-2-methylpropyl)-3,5-dimethylpiperazin-1-yl)methyl)benzamide (Compound 488): To a stirred solution of Boc compound 8b (1 eq) in 1,4-dioxane (5 vol.), 4 M HCl in dioxane (5 vol.) was added. The resulting reaction mass was stirred at room temperature for 1 h. The progress of the reaction was monitored TLC. After completion, the reaction mixture was concentrated under reduced pressure. The residue was basified with sat. NaHCO₃ solution and extracted with ethyl acetate. The organic layers were separated, washed with water and brine, dried over Na₂SO₄ and concentrated. The crude residue was purified by prep. HPLC to provide the desired compound.

¹H NMR (400 MHz, DMSO-d6) δ 9.61 (s, 1H), 7.92 (d, J=7.2 Hz, 2H), 7.42 (d, J=7.6 Hz, 2H), 7.15 (d, J=8.0 Hz, 1H), 6.96 (t, J=7.6 Hz, 1H), 6.77 (d, J=8.0 Hz, 1H), 6.58 (t, J=7.2 Hz, 1H), 4.88 (s, 2H), 3.88 (s, 1H), 3.47 (s, 2H), 2.71-2.69 (m, 2H), 2.47-2.37 (m, 4H), 2.07-2.05 (m, 2H), 1.07-0.99 (m, 12H); LCMS Calculated for C₂₄H₃₄N₄O₂: 410.27; Observed: 411.10(M+1)⁺.

### Synthetic Scheme for Compound 477, Compound 477-Isomer-I, Compound 477-Isomer-II and Compound 478, Compound 478-Isomer-I, Compound 478-Isomer-II

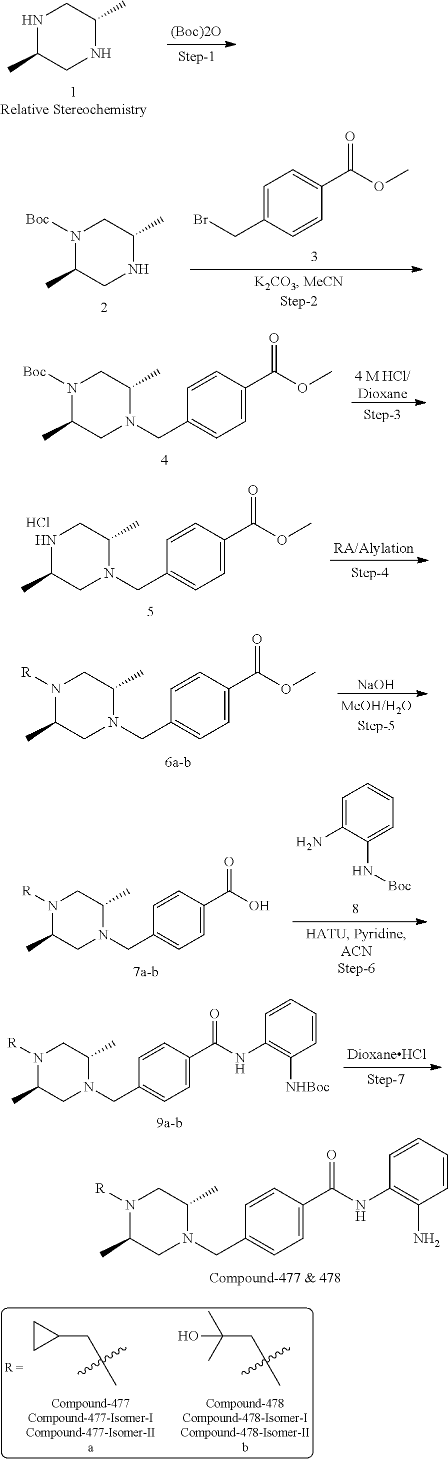

Step 1: Synthesis of tert-butyl (2R,5S)-2,5-dimethylpiperazine-1-carboxylate (2): To a stirred solution of compound 1 (0.5 g, 1 eq) in DCM (15 mL) at 0° C., Boc-anhydride (0.478 g, 0.5 eq) dissolved in DCM was added drop wise. The reaction mixture was stirred at room temperature for 24 h. the reaction progress was monitored by TLC. After completion, the reaction mixture was diluted with water and extracted with DCM. The organic layers were separated, washed with water and brine, dried over Na₂SO₄ and evaporated to afford the desired compound 2.

Step 2: Synthesis of tert-butyl (2R,5S)-4-(4-(methoxycarbonyl)benzyl)-2,5-dimethylpiperazine-1-carboxylate (4): To a stirred solution of compound 2 (0.85 g, 1 eq) and compound 3 (0.91 g, 1 eq) in ACN (10 mL), potassium carbonate (1.65 g, 3 eq) was added. The reaction mixture was stirred at room temperature for 16 h. The progress of reaction was monitored by TLC. After completion, the reaction mixture was diluted with water and extracted with ethyl acetate. The combined organic extracts were washed with water, brine, dried over anhydrous Na₂SO₄, filtered and concentrated under reduced pressure to provide a crude residue which was purified by silica gel column chromatography to afford compound 4.

Step 3: Synthesis of methyl 4-(((2S,5R)-2,5-dimethylpiperazin-1-yl)methyl)benzoate hydrochloride (5): To a stirred solution of Boc compound 4 (0.6 g, 1 eq) in 1,4-dioxane (2 mL) was added 4 M HCl in dioxane (1 mL) reaction was stirred at room temperature for 1 h. After completion of reaction, the reaction mixture was concentrated and the resulting residue was triturated with n-pentane and dried under vacuum to give the desired compound 5 as HCl salt.

Step 4: Synthesis of compound 6a for compound 477: To a stirred solution of amine compound 5 (1 eq) and aldehyde (1.2 eq) in DCM (10 vol.), acetic acid (6 eq) was added and stirred at room temperature for 30 min. To this, sodium triacetoxyborohydride (STAB) (3 eq) was added at room temperature. The resulting reaction mixture was stirred at room temperature for overnight; the reaction progress was monitored by TLC and LCMS. After completion, the reaction mixture was quenched with sat. NaHCO₃ solution and extracted with DCM. The organic layers were separated, washed with water and brine, dried over Na₂SO₄ and evaporated to get the crude product which was purified by silica gel column chromatography to afford the desired compound 6a.

Step 4: Synthesis of compound 6b for compound 478: To a solution of compound 5 (1 eq) in ethanol (10 vol.), TEA (3 eq) and 2,2-dimethyloxirane (1.5 eq) were added and the reaction mixture was heated at 80° C. for 12 h. The progress of reaction was monitored by TLC. After completion, the reaction mixture was allowed to cool, concentrated to give a crude compound which was purified by silica gel column chromatography to afford the desired compound 6b.

| No | Structure |
|---|---|
| 1 | 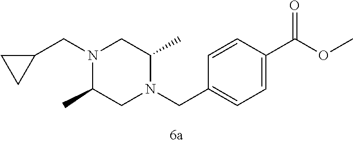 [SMILES: COC(=O)c1ccc(CN2C[C@@H](C)N(CC3CC3)C[C@@H]2C)cc1] |
| 2 | 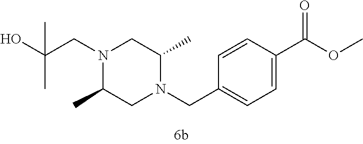 [SMILES: COC(=O)c1ccc(CN2C[C@@H](C)N(CC(C)(C)O)C[C@@H]2C)cc1] |

Step 5: Synthesis of compounds 7a-b: To stirred solution of ester compound in Methanol: Water (1:1) was added NaOH (1.5 eq) at room temperature. The above mixture was heated to 90° C. for 5 h. The progress of the reaction was monitored by TLC. After completion of reaction, the reaction mixture was concentrated and the resulting residue partioned between diethyl ether and water. The aqueous layer was neutralized to pH=7 using 1 N HCl at 0° C. The solid obtained was filtered, washed with water and dried under vacuum to provide the desired compound.

| No | Structure |
|---|---|
| 1 | 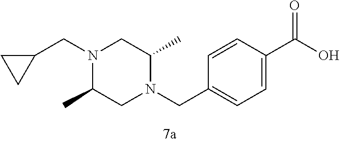 [SMILES: C[C@@H]1CN(Cc2ccc(C(=O)O)cc2)[C@@H](C)CN1CC1CC1] |
| 2 | 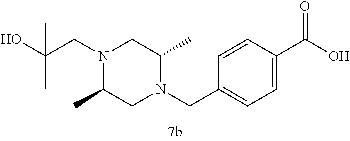 [SMILES: C[C@@H]1CN(Cc2ccc(C(=O)O)cc2)[C@@H](C)CN1CC(C)(C)O] |

Step 6: Synthesis of compounds 9a-b: To a stirred solution of acid compound (1 eq) and amine (1.2 eq) in ACN, pyridine (6 eq) and HATU (1.5 eq) were added at room temperature. The reaction mixture was stirred at 90° C. for overnight; the reaction progress was monitored by TLC and LCMS. After completion, the reaction mixture was concentrated and resulting residue was portioned between water and ethyl acetate. The organic layers were separated, washed with water and 1% HCl to remove traces of pyridine, dried over Na₂SO₄ and concentrated. The crude residue was purified by silica gel column chromatography to provide the desired compound.

| No | Structure |
|---|---|
| 1 | 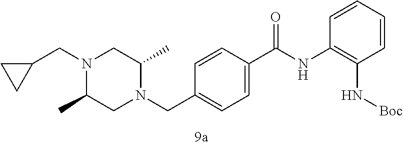 [SMILES: C[C@@H]1CN(Cc2ccc(C(=O)Nc3ccccc3NC(=O)OC(C)(C)C)cc2)[C@@H](C)CN1CC1CC1] |
| 2 | 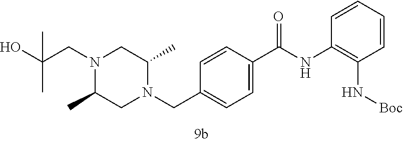 [SMILES: C[C@@H]1CN(Cc2ccc(C(=O)Nc3ccccc3NC(=O)OC(C)(C)C)cc2)[C@@H](C)CN1CC(C)(C)O] |

Step 7: Synthesis of N-(2-aminophenyl)-4-(((2S,5R)-4-(cyclopropylmethyl)-2,5-dimethylpiperazin-1-yl)methyl)benzamide (Compound 477): To a stirred solution of Boc compound 9a (, 1 eq) in 1,4-dioxane (5vol.), 4 M HCl in dioxane (5 vol.) was added. The resulting reaction mass was stirred at room temperature for 1 h. The progress of the reaction was monitored TLC. After completion, the reaction mixture was concentrated under reduced pressure. The residue was basified with sat. NaHCO₃ solution and extracted with ethyl acetate. The organic layers were separated, washed with water and brine, dried over Na₂SO₄ and concentrated. The crude residue was purified by silica gel column chromatography /prep. HPLC to provide the desired compound.

¹H NMR (400 MHz, DMSO-d6) δ 9.61 (s, 1H), 7.93 (d, J=8.0 Hz, 2H), 7.42 (d, J=8.0 Hz, 2H), 7.16 (d, J=7.8 Hz, 1H), 6.97 (t, J=7.6 Hz, 1H), 6.78 (d, J=7.6 Hz, 1H), 6.60 (t, J=7.6 Hz, 1H), 4.88 (s, 2H), 4.07 (d, J=13.8 Hz, 1H), 3.10 (d, J=13.8 Hz, 1H), 2.97 (dd, J=11.5, 2.8 Hz, 1H), 2.59-2.56 (m, 2H), 2.42-2.29 (m, 1H), 2.31-2.19 (m, 1H), 2.10-1.97 (m, 2H), 1.77 (t, J=10.6 Hz, 1H), 1.10 (d, J=6.0 Hz, 3H), 0.89-0.74 (m, 4H), 0.53-0.36 (m, 2H), 0.07-0.05 (m, 2H); LCMS Calculated for C₂₄H₃₂N₄O: 392.26; Observed: 393.30 (M+1)⁺.

Step 6: Synthesis of compound 9 compound 477-Isomer-I, compound 477-Isomer-II and compound 478-Isomer-I, compound 478-Isomer-II: To a stirred solution of compound 7 (1 eq) and compound 8 (1.2 eq) in DMF (5 mL), DIPEA (3 eq) was added and stirred for 10 min. To this, HATU (0.534 g, 1.5 eq) was added and the reaction mixture was stirred at room temperature for overnight, the reaction progress was monitored by TLC and LCMS. After completion of reaction, the reaction mixture was portioned between ethyl acetate and water. The organic layers were separated, washed with water and brine, dried over Na₂SO₄ and evaporated to get the crude product which was purified by silica gel column chromatography to afford the desired compound.

| No | Structure |
|---|---|
| 1 | 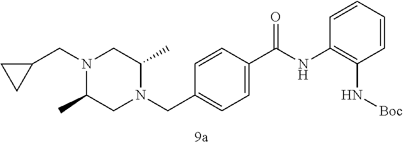 [SMILES: C[C@@H]1CN(Cc2ccc(C(=O)Nc3ccccc3NC(=O)OC(C)(C)C)cc2)[C@@H](C)CN1CC1CC1] |
| 2 | 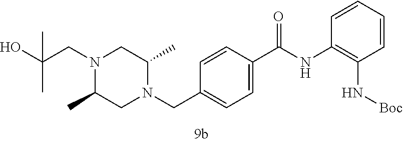 [SMILES: C[C@@H]1CN(Cc2ccc(C(=O)Nc3ccccc3NC(=O)OC(C)(C)C)cc2)[C@@H](C)CN1CC(C)(C)O] |

Step 7: Synthesis of N-(2-aminophenyl)-4-((4-(cyclopropylmethyl)-2,5-dimethylpiperazin-1-yl)methyl)benzamide (Compound 477-Isomer-II): To a stirred solution of Boc compound 9a (1 eq) in 1,4-dioxane (5 vol.), 4 M HCl in dioxane (5 vol.) was added. The resulting reaction mass was stirred at room temperature for 1 h. The progress of the reaction was monitored TLC. After completion, the reaction mixture was concentrated under reduced pressure. The residue was basified with sat. NaHCO₃ solution and extracted with ethyl acetate. The organic layers were separated, washed with water and brine, dried over Na₂SO₄ and concentrated. The crude residue was purified by chiral prep. HPLC using column YMC CHIRALART CELLULOSE-SC, 250 mm×4.6 mm, 5 μm column and delivered as Compound 477-Isomer-I as free base (RT is 17.19) and Compound 477-Isomer-II as free base (RT is 25.46), where the absolute stereochemistry has yet to be confirmed.

Compound 477-Isomer-Has free base, ¹H NMR (400 MHz, DMSO-d6) δ 9.60 (s, 1H), 7.92 (d, J=7.8 Hz, 2H), 7.40 (d, J=7.9 Hz, 2H), 7.14 (d, J=7.8 Hz, 1H), 7.00-6.91 (m, 1H), 6.76 (d, J=8.0 Hz, 1H), 6.63-6.54 (m, 1H), 4.87 (s, 2H), 4.06 (d, J=13.8 Hz, 1H), 3.09 (d, J=13.7 Hz, 1H), 2.97 (d, J=11.2 Hz, 1H), 2.35-2.26 (m, 4H), 2.06-2.04 (m, 1H), 1.77 (t, J=10.6 Hz, 1H), 1.20-1.05 (m, 3H), 0.85-0.83 (m, 4H), 0.46-0.40 (m, 2H), 0.05-0.04 (m, 2H), 1H merged in solvent peak; LCMS Calculated for C₂₄H₃₂N₄O: 392.26; Observed: 393.35 (M+1)⁺.

Compound 477-Isomer-I as free base, ¹H NMR (400 MHz, DMSO-d6) δ 9.61 (s, 1H), 7.91 (d, J=7.2 Hz, 2H), 7.41 (d, J=8.0 Hz, 2H), 7.15 (d, J=7.6 Hz, 1H),7.01-6.91 (m, 1H), 6.76 (d, J=8.0 Hz, 1H), 6.58 (t, J=7.5 Hz, 1H), 4.87 (s, 2H), 4.06 (d, J=13.8 Hz, 1H), 3.08 (d, J=13.8 Hz, 1H), 2.98-2.95 (m, 1H), 2.32-2.24 (m, 2H), 2.10-1.98 (m, 2H), 1.77-1.73 (m, 1H), 1.24-1.03 (m, 3H), 0.88-0.75 (m, 4H), 0.52-0.35 (m, 2H), 0.11-0.03 (m, 2H), 2H merged in solvent peak; LCMS Calculated for C₂₄H₃₂N₄O: 392.26; Observed: 393 (M+1)⁺.

Step 7: Synthesis of N-(2-aminophenyl)-4-(((2S,5R)-4-(2-hydroxy-2-methylpropyl)-2,5-dimethylpiperazin-1-yl)methyl)benzamide (Compound 478): To a stirred solution of Boc compound 9b (1 eq) in 1,4-dioxane (5 vol.), 4 M HCl in dioxane (5 vol.) was added. The resulting reaction mass was stirred at room temperature for 1 h. The progress of the reaction was monitored TLC. After completion, the reaction mixture was concentrated under reduced pressure. The residue was basified with sat. NaHCO₃ solution and extracted with ethyl acetate. The organic layers were separated, washed with water and brine, dried over Na₂SO₄ and concentrated. The crude residue was purified by silica gel column chromatography/prep. HPLC to provide the desired compound.

¹H NMR (400 MHz, DMSO-d6) δ 9.61 (s, 1H), 7.93 (d, J=8.0 Hz, 2H), 7.42 (d, J=8.0 Hz, 2H), 7.20-7.13 (m, 1H), 6.98-6.95 (m, 1H), 6.78 (d, J=8.0 Hz, 1H), 6.64-6.55 (m, 1H), 4.88 (s, 2H), 4.06-3.94 (m, 2H), 3.18-3.05 (m, 2H), 2.39-2.27 (m, 2H), 2.11-2.00 (m, 1H), 1.94-1.91 (m, 1H), 1.80 (t, J=10.4 Hz, 1H), 1.07-1.05 (m, 9H), 0.85 (d, J=6.4 Hz, 3H), 1H merged in solvent peak; LCMS Calculated for C₂₄H₃₄N₄O₂: 410.27; Observed: 411.25 (M+1)⁺.

Step 7: Synthesis of N-(2-aminophenyl)-4-((4-(2-hydroxy-2-methylpropyl)-2,5-dimethylpiperazin-1-yl)methyl)benzamide (Compound 478-Isomer-I): To a stirred solution of Boc compound 9b(1 eq) in 1,4-dioxane (5 vol.), 4 M HCl in dioxane (5 vol.) was added. The resulting reaction mass was stirred at room temperature for 1 h. The progress of the reaction was monitored TLC. After completion, the reaction mixture was concentrated under reduced pressure. The residue was basified with sat. NaHCO₃ solution and extracted with ethyl acetate. The organic layers were separated, washed with water and brine, dried over Na₂SO₄ and concentrated. The crude residue was purified by chiral prep. HPLC using column YMC Chiral AMYLOSE-SA, 250 mm×4.6 mm, 5 μm column and delivered as Compound 478-Isomer-I as free base (RT is 10.52) and Compound 478-Isomer-II as free base (RT is 13.77), where the absolute stereochemistry has yet to be confirmed.

Compound 478-Isomer-I as free base ¹H NMR (400 MHz, DMSO-d6) δ 9.62 (s, 1H), 7.93 (d, J=7.8 Hz, 2H), 7.42 (d, J=7.8 Hz, 2H), 7.22-7.12 (m, 1H), 7.01-6.92 (m, 1H), 6.78-6.76 (m, 1H), 6.61-6.57 (m, 1H), 4.88 (s, 2H), 4.06-3.95 (m, 2H), 3.20-3.05 (m, 2H), 2.43-2.34 (m, 1H), 2.26-2.24 (m, 1H), 2.08-2.03 (m, 1H), 1.95-1.91 (m, 1H), 1.83-1.77 (m, 1H), 1.10-1.02 (m, 9H), 0.84 (d, J=6.1 Hz, 3H), 1H merged in solvent peak; LCMS Calculated for C₂₄H₃₄N₄O₂: 410.27; Observed: 411.15 (M+1)⁺.

Compound 478-Isomer-II as free base ¹H NMR (400 MHz, DMSO-d6) δ 9.61 (s, 1H), 7.92 (d, J=7.8 Hz, 2H), 7.41 (d, J=7.8 Hz, 2H), 7.15 (d, J=7.6 Hz, 1H), 7.00-6.91 (m, 1H), 6.77 (d, J=8.0 Hz, 1H), 6.58 (t, J=7.6 Hz, 1H), 4.88 (s, 2H), 4.03-3.99 (m, 2H), 3.15-3.08 (m, 2H), 2.52-2.49 (m, 1H), 2.38-2.36 (m, 1H), 2.26-2.24 (m, 1H), 2.06-2.05 (m, 1H), 1.95-1.91 (m, 1H), 1.80-1.78 (m, 1H), 1.09-1.02 (m, 9H), 0.84 (d, J=6.0 Hz, 3H); LCMS Calculated for C₂₄H₃₄N₄O₂: 410.27; Observed: 411.15 (M+1)⁺.

### Synthetic Scheme for Compound 356 and Compound 359

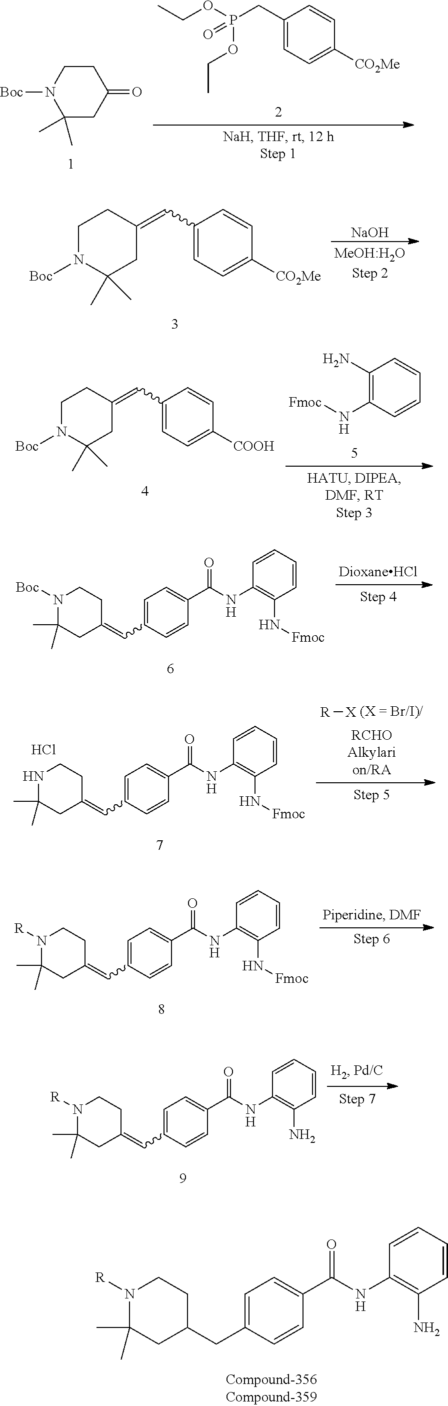

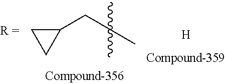

Step 1: Synthesis of tert-butyl 4-(4-(methoxycarbonyl)benzylidene)-2,2-dimethylpiperidine-1-carboxylate (3): To a stirred solution of compound 2 (3 g, 1.2 eq) in dry THF (20 mL) at 0° C., NaH (60%, 0.506 g, 1.2 eq) was added slowly and stirred at same temperature for 30 min. To this solution, compound 1 (2 g, 1 eq) dissolved in dry THF was added slowly. The resulting reaction mixture was stirred at room temperature for 12 h. The progress of the reaction was monitored by TLC. After completion, the reaction was quenched with water and extracted with ethyl acetate. The organic layers were separated, dried over Na₂SO₄ and concentrated. The crude residue was purified by silica gel column chromatography to provide the mixture of desired compound 3 (Cis/Trans mixture).

Step 2: Synthesis of 4-((1-(tert-butoxycarbonyl)-2,2-dimethylpiperidin-4-ylidene)methyl)benzoic acid (4): To stirred solution of mixture of compound 3 (2.5 g, 1 eq) in Methanol: Water (1:1, 20 mL), NaOH (0.417 g, 1.5 eq) was added at room temperature. The above mixture was at room temperature for 2 h. The progress of the reaction was monitored by TLC. After completion of reaction, the reaction mixture was concentrated and the resulting residue was neutralized to pH=7 using 1 N HCl at 0° C. The solid obtained was filtered, washed with water and dried under vacuum to provide the mixture of desired compound.

Step 3: Synthesis of compound 6: To a stirred solution of compound 4 (1 g, 1 eq) and amine 5 (0.956 g, 1 eq) in DMF, DIPEA (1.24 mL, 2.5 eq) was added and stirred for 10 min. To this, HATU (1.65 g 1.5 eq) was added and the reaction mixture was stirred at room temperature for overnight, the reaction progress was monitored by TLC and LCMS. After completion, the reaction mixture was portioned between ethyl acetate and water. The organic layers were separated, washed with water and brine, dried over Na₂SO₄ and evaporated to get the crude product which was purified by silica gel column chromatography to afford the mixture of desired compound.

Step 4: Synthesis of compound 7: To a stirred solution of compound 6 (1.2 g, 1 eq) in 1,4-dioxane, 4 M HCl in dioxane was added. The resulting reaction mass was stirred at room temperature for 1 h. After completion, the reaction mixture was concentrated under reduced pressure and the resulting residue was triturated with diethyl ether and dried under vacuum to give the mixture of title compound 7 as HCl salt.

Step 5: Synthesis of compound 8: To a stirred solution of amine compound 7 (1 eq) and corresponding aldehyde (1.5 eq) in DCM, acetic acid (6 eq) was added and stirred at room temperature for 30 min. To this, sodium triacetoxyborohydride (STAB) (3 eq) was added at room temperature. The resulting reaction mixture was stirred at room temperature for overnight; the reaction progress was monitored by TLC and LCMS. After completion, the reaction mixture was quenched with sat. NaHCO₃ solution and extracted with DCM. The organic layers were separated, washed with water and brine, dried over Na₂SO₄ and evaporated to get the crude product which was purified by silica gel column chromatography to afford the mixture of desired compound.

Step 6: Synthesis of compound 9: A mixture of compound 7 (0.32 g, 1 eq) and 20% piperidine in DMF (2 mL) was stirred at room temperature for 15 min. The progress of the reaction was monitored by TLC. After completion, the reaction mixture diluted with ice cold water. The solid obtained was filtered, washed with water and dried under vacuum to provide the mixture of desired compound 9.

| No | Structure |
|---|---|
| 1 | 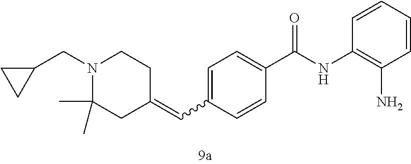 [SMILES: CC1(C)CC(=Cc2ccc(C(=O)Nc3ccccc3N)cc2)CCN1CC1CC1] |
| 2 | 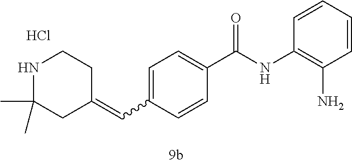 [SMILES: CC1(C)CC(=Cc2ccc(C(=O)Nc3ccccc3N)cc2)CCN1 ; Cl] |

Step 7: Synthesis of N-(2-aminophenyl)-4-((1-(cyclopropylmethyl)-2,2-dimethylpiperidin-4-yl)methyl)benzamide (Compound 356): To a stirred solution of compound 9a (0.05 g, 1 eq) in methanol (5 mL), 10% Pd/C (10% w/w of substrate, 30 mg) was added and the reaction mixture was stirred under hydrogen atmosphere (balloon pressure) at room temperature for 1 h. The progress of the reaction was monitored by TLC. After completion, the reaction mixture was filtered through a pad of celite, the filtrate was evaporated under reduced pressure and the resulting residue was triturated with diethyl ether and n-pentane and then dried under vacuum to afford the title compound.

¹H NMR (400 MHz, DMSO-d6) δ 9.59 (s, 1H), 7.90 (d, J=7.2 Hz, 2H), 7.28 (d, J=7.2 Hz, 2H), 7.15 (d, J=8.0 Hz, 1H), 6.96 (t, J=7.6 Hz, 1H), 6.78 (d, J=8.0 Hz, 1H), 6.59 (t, J=7.6 Hz, 1H), 4.88 (s, 2H), 2.92-2.90 (m, 2H), 2.62-2.59 (m, 2H), 2.33-2.32 (m, 1H), 2.16-2.14 (m, 2H), 1.75-1.72 (m, 1H), 1.55-1.51 (m, 1H), 1.32-1.07 (m, 5H), 0.80-0.78(m, 4H), 0.45-0.34 (m, 2H), 0.07-0.04 (m, 2H); LCMS Calculated for C₂₅H₃₃N₃O: 391.26; Observed: 391.95 (M+1)⁺.

Step 7: Synthesis of N-(2-aminophenyl)-4-((2,2-dimethylpiperidin-4-yl)methyl)benzamide (Compound 359): To a stirred solution of compound 9b (0.08 g, 1 eq) in methanol (5 mL), 10% Pd/C (10% w/w of substrate, 40 mg) was added and the reaction mixture was stirred under hydrogen atmosphere (balloon pressure) at room temperature for 1 h. The progress of the reaction was monitored by TLC. After completion, the reaction mixture was filtered through a pad of celite, the filtrate was evaporated under reduced pressure and the resulting residue was triturated with diethyl ether and n-pentane and then dried under vacuum to afford the title compound.

¹H NMR (400 MHz, DMSO-d6) δ 9.63 (s, 1H), 8.41 (s, 1H), 7.92 (d, J=7.8 Hz, 2H), 7.31 (d, J=7.8 Hz, 2H), 7.16 (d, J=7.8 Hz, 1H), 6.97 (t, J=7.6 Hz, 1H), 6.78 (d, J=7.9 Hz, 1H), 6.60 (t, J=7.5 Hz, 1H), 4.87-4.84 (m, 2H), 3.36-3.27 (m, 1H), 2.99-2.82 (m, 3H), 2.56-2.49 (m, 2H), 2.05-1.90 (m, 1H), 1.66-1.48 (m, 2H), 1.24-1.10 (m, 6H); LCMS Calculated for C₂₁H₂₇N₃O: 337.22; Observed: 338.10(M+1)⁺.

### Synthetic scheme for Compound 357

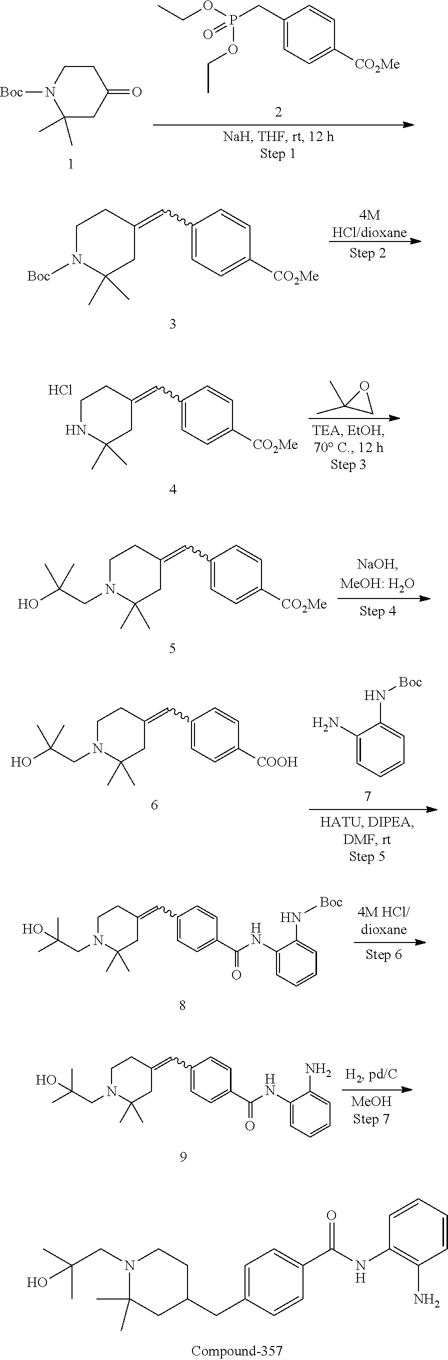

Step 2: Synthesis of methyl 4-((2,2-dimethylpiperidin-4-ylidene)methyl)benzoate (4): To a stirred solution of compound 3 (0.85 g, 1 eq) in 1,4-dioxane (5 mL), 4 M HCl in dioxane (10 mL) was added. The resulting reaction mass was stirred at room temperature for 1 h. After completion, the reaction mixture was concentrated under reduced pressure and the resulting residue was triturated with diethyl ether and dried under vacuum to give the mixture of title compound 4 (Cis/Trans mixture) as HCl salt.

Step 3: Synthesis of methyl 4-((1-(2-hydroxy-2-methylpropyI)-2,2-dimethylpiperidin-4-ylidene)methyl)benzoate (5): To a solution of compound 4 (0.35 g, 1 eq) in ethanol (10 mL), TEA (0.567 mL, 3 eq) and 2,2-dimethyloxirane (0.42 mL, 3.5 eq) were added and the reaction mixture was heated at 70° C. for 12 h. The progress of reaction was monitored by TLC. After completion, the reaction mixture was allowed to cool, concentrated to afford the mixture of desired compound 5.

Step 4: Synthesis of 4-((1-(2-hydroxy-2-methylpropyl)-2,2-dimethylpiperidin-4-ylidene)methyl)benzoic acid (6): To stirred solution of ester compound 5 (0.4 g, 1 eq) in Methanol: Water (1:1, 10 mL), NaOH (0.073 g, 1.5 eq) was added at room temperature. The above mixture was heated to 70° C. for 3 h. The progress of the reaction was monitored by TLC. After completion of reaction, the reaction mixture was concentrated and the resulting residue was washed with diethyl ether followed by treatment with water. The aqueous layer was neutralized to pH=7 using 1 N HCl at 0° C. The solid obtained was filtered, washed with water and dried under vacuum to provide the mixture of desired compound 6.

Step 5: Synthesis of tert-butyl (2-(4-((1-(2-hydroxy-2-methylpropyl)-2,2-dimethylpiperidin-4-ylidene)methyl)benzamido)phenyl)carbamate (8): To a stirred solution of compound 6 (0.38 g, 1 eq) and compound 7 (0.299 g, 1.2 eq) in DMF (7 mL), DIPEA (0.515 mL, 2.5 eq) was added and stirred for 10 min. To this, HATU (0.683 g, 1.5 eq) was added and the reaction mixture was stirred at room temperature for overnight, the reaction progress was monitored by TLC and LCMS. After completion, the reaction mixture was portioned between ethyl acetate and water. The organic layers were separated, washed with water and brine, dried over Na₂SO₄ and evaporated to get the crude product which was purified by silica gel column chromatography to afford the mixture of desired compound 8.

Step 6: Synthesis of N-(2-aminophenyl)-4-((1-(2-hydroxy-2-methylpropyl)-2,2-dimethylpiperidin-4-ylidene)methyl)benzamide (9): To a stirred solution of compound 8 (0.5 g, 1 eq) in 1,4-dioxane, 4 M HCl in dioxane was added. The resulting reaction mass was stirred at room temperature for 1 h. After completion, the reaction mixture was concentrated under reduced pressure and the resulting residue was triturated with diethyl ether and dried under vacuum to give the mixture of title compound 9 as HCl salt.

Step 7: Synthesis of N-(2-aminophenyl)-4-((1-(2-hydroxy-2-methylpropyl)-2,2-dimethylpiperidin-4-yl)methyl)benzamide (Compound 357): To a stirred solution of compound 9 (0.09 g, 1 eq) in methanol (5 mL), 10% Pd/C (10% w/w of substrate, 40 mg) was added and the reaction mixture was stirred under hydrogen atmosphere (balloon pressure) at room temperature for 1 h. The progress of the reaction was monitored by TLC. After completion, the reaction mixture was filtered through a pad of celite, the filtrate was evaporated under reduced pressure and the resulting residue was triturated with diethyl ether and n-pentane and then dried under vacuum to afford the title compound.

¹H NMR (400 MHz, DMSO-d6) δ 9.59 (s, 1H), 7.89 (d, J=7.9 Hz, 2H), 7.28 (d, J=7.9 Hz, 2H), 7.15 (d, J=7.9 Hz, 1H), 7.01-6.92 (m, 1H), 6.77 (d, J=7.2 Hz, 1H), 6.64-6.55 (m, 1H), 4.88 (s, 2H), 3.90-3.88 (m, 1H), 2.92-2.89 (m, 1H), 2.31-2.28 (m, 1H), 1.81-1.78 (m, 2H), 1.45-1.29 (m, 4H), 1.28-1.01 (m, 10H), 0.98 (s, 3H), 2H merged in solvent peak; LCMS Calculated for C₂₅H₃₅N₃O₂: 409.27; Observed: 410.15(M+1)⁺.

### Synthetic Scheme for Compound 379

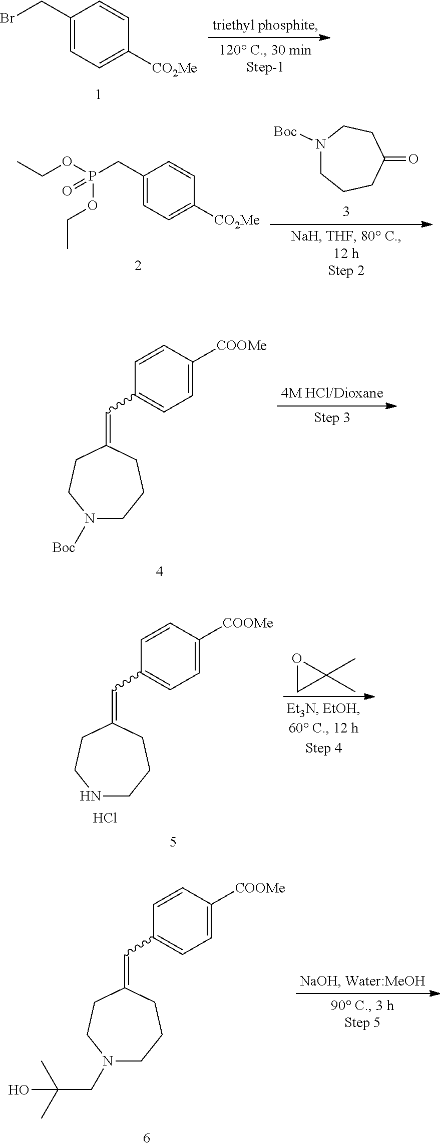

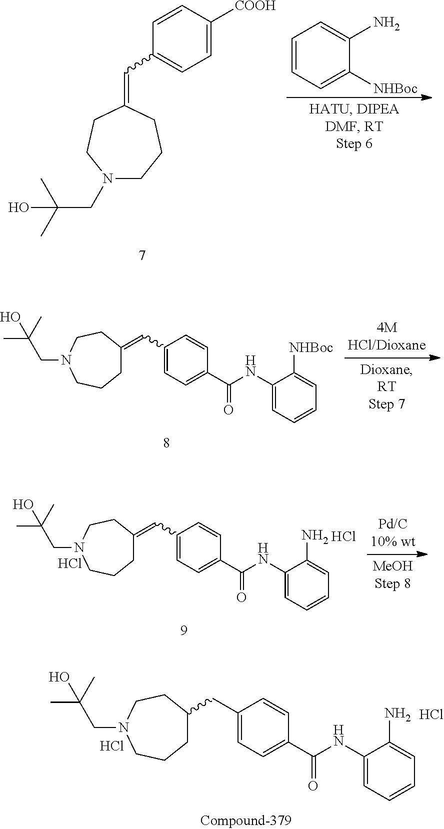

Step 1: Synthesis of methyl 4-((diethoxyphosphoryl)methyl)benzoate (2): A mixture of compound 1 (10 g, 1 eq) and triethyl phosphite (8.96 mL, 1.2 eq) was heated in sealed tube at 120° C. for 30 min. The progress of the reaction was monitored by TLC. After completion, the reaction mixture was concentrated under reduced pressure to afford the crude compound 2.

Step 2: Synthesis of tert-butyl 4-(4-(methoxycarbonyl)benzylidene)azepane-1-carboxylate (4): To a stirred solution of compound 2 (0.5 g, 1 eq) in dry THF (10 mL) at 0° C., NaH (60%, 0.063 g, 1.5 eq) was added slowly and stirred at same temperature for 30 min. To this solution, compound 3 (0.261 g, 0.7 eq) dissolved in dry THF was added slowly. The resulting reaction mixture was stirred at 80° C. for 12 h. The progress of the reaction was monitored by TLC. After completion, the reaction was quenched with water and extracted with ethyl acetate. The organic layers were separated, washed with water and dried over Na₂SO₄ and concentrated. The crude residue was purified by silica gel column chromatography to provide the desired compound.

Step 3: Synthesis of methyl 4-(azepan-4-ylidenemethyl)benzoate hydrochloride (5): To a stirred solution of Boc compound 4 (2.4 g, 1 eq) in 1,4-dioxane (10 mL), 4 M HCl in dioxane (4 mL) was added and the reaction was stirred at room temperature for 1 h. After completion, the reaction mixture was concentrated and the resulting residue was triturated with n-pentane and dried under vacuum to give the desired compound 5 as HCl salt.

Step 4: Synthesis of methyl 4-((1-(2-hydroxy-2-methylpropyl)azepan-4-ylidene)methyl)benzoate (6): To a solution of compound 5 (1.6 g, 1 eq) in ethanol (20 mL), TEA (2.75 mL, 3 eq) and 2,2-dimethyloxirane (0.47 g, 1 eq) were added at room temperature and the reaction mixture was heated at 60° C. for 12 h. The progress of reaction was monitored by TLC. After completion, the reaction mixture was allowed to cool, concentrated to give a crude compound which was purified by silica gel column chromatography.

Step 5: Synthesis of 4-((1-(2-hydroxy-2-methylpropyl)azepan-4-ylidene)methyl)benzoic acid (7): To stirred solution of ester compound 6 (1.9 g, 1 eq) in Methanol: Water (1:1, 10 mL), NaOH (0.36 g, 1.5 eq) was added at room temperature. The above mixture was heated to 70° C. for 12 h. The progress of the reaction was monitored by TLC. After completion of reaction, the reaction mixture was concentrated and the resulting residue was washed with diethyl ether followed by treatment with water. The aqueous layer was neutralized to pH=7 using 1 N HCl at 0° C. The solid obtained was filtered, washed with water and dried under vacuum to provide the desired compound 7.

Step 6: Synthesis of tert-butyl (2-(4-((1-(2-hydroxy-2-methylpropyl)azepan-4-ylidene)methyl)benzamido)phenyl)carbamate (8): To a stirred solution of compound 7 (1.6 g, 1 eq) and tert-butyl (2-aminophenyl)carbamate (1.1 g, 1 eq) in DMF (10 mL), DIPEA (2.28 mL, 2.5 eq) was added and stirred for 10 min. To this, HATU (3 g, 1.5 eq) was added and the reaction mixture was stirred at room temperature for overnight, the reaction progress was monitored by TLC and LCMS. After completion, the reaction mixture was partitioned between ethyl acetate and water. The organic layers were separated, washed with water and brine, dried over Na₂SO₄ and evaporated to get the crude product which was purified by silica gel column chromatography to afford the desired compound 8.

Step 7: Synthesis of N-(2-aminophenyl)-4-((1-(2-hydroxy-2-methylpropyl)azepan-4-ylidene)methyl)benzamide (9): To a stirred solution of Boc compound 8 (1 g, 1 eq) in 1,4-dioxane (5 mL), 4 M HCl in dioxane (2 mL) was added and stirred at room temperature for 1 h. After completion, the reaction mixture was concentrated and the resulting residue was triturated with n-pentane and dried under vacuum to provide the desired compound 9 as HCl salt.

Step 8: Synthesis of N-(2-aminophenyl)-4-((1-(2-hydroxy-2-methylpropyl)azepan-4-yl)methyl)benzamide dihydrochloride (Compound-379): To a stirred solution of 9 (0.2 g, 1 eq) in methanol (10 mL), 10% Pd/C (20 mg) was added and the reaction mixture was stirred under hydrogen atmosphere (balloon pressure) at room temperature for 5 h. The progress of the reaction was monitored by TLC. After completion, the reaction mixture was filtered through a pad of celite, the filtrate was evaporated under reduced pressure and the resulting residue was triturated with diethyl ether and n-pentane and then dried under vacuum to afford the title compound.

¹H NMR (400 MHz, DMSO-d6) δ 10.13 (s, 1H), 9.40 (s, 1H), 8.01 (d, J=7.8 Hz, 2H), 7.39 (d, J=8.0 Hz, 1H), 7.32 (d, J=7.6 Hz, 2H), 7.18-7.16 (m, 2H), 7.05-7.03 (m, 1H), 3.58-3.00 (m, 7H), 2.61-2.57 (m, 2H), 1.93-1.61 (m, 7H), 1.23 (s, 6H); LCMS Calculated for C₂₄H₃₃N₃O₂ (free base): 395.26; Observed: 396.30 (M+1)⁺.

### Synthetic Scheme for Compound 181 and Compound 472

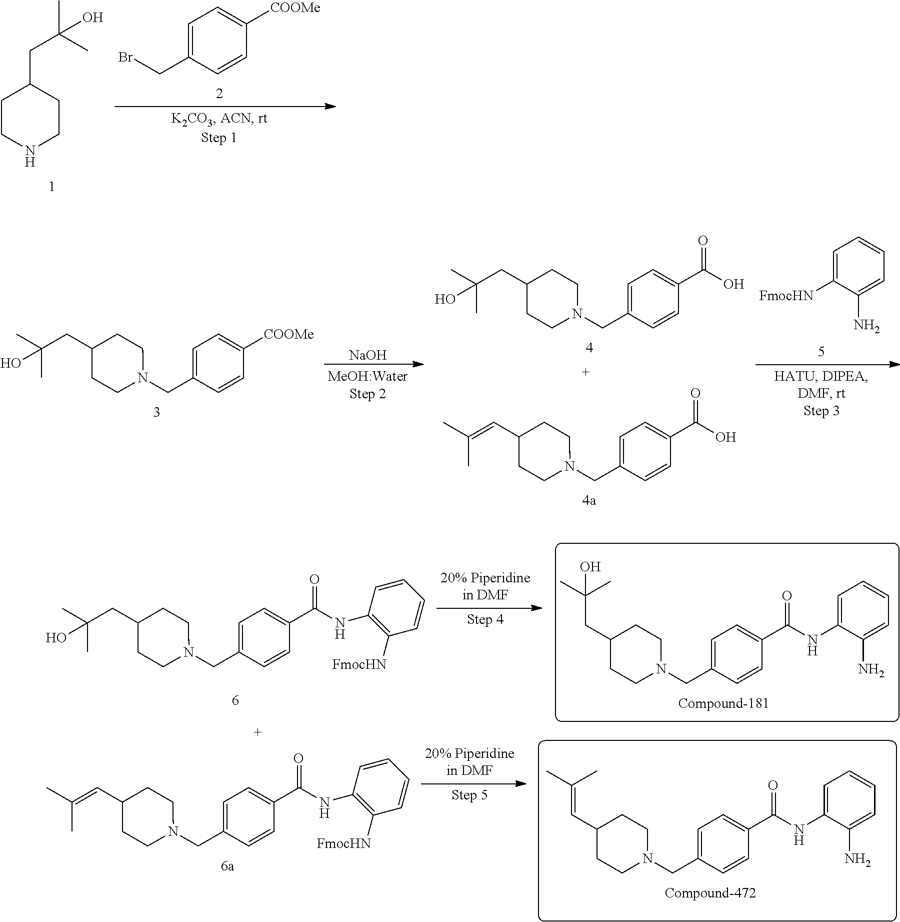

Step 1: Synthesis of methyl 4-((4-(2-hydroxy-2-methylpropyl)piperidin-1-yl)methyl)benzoate (3): To a stirred solution of compound 1 (1 g, 1 eq) and compound 2 (1.02 g, 1 eq) in ACN (20 mL), potassium carbonate (2.6 g, 3 eq) was added. The reaction mixture was stirred at room temperature for 16 h. The progress of reaction was monitored by TLC. After completion, the reaction mixture was diluted with water and extracted with ethyl acetate. The combined organic extracts were washed with water, brine, dried over anhydrous Na₂SO₄, filtered and concentrated under reduced pressure to afford the crude product which was purified by silica gel column chromatography to afford the desired compound 3.

Step 2: Synthesis of 4-((4-(2-hydroxy-2-methylpropyl)piperidin-1-yl)methyl)benzoic acid and 4-((4-(2-methylprop-1-en-1-yl)piperidin-1-yl)methyl)benzoic acid (4 and 4a): To stirred solution of ester compound 3 (0.3 g, 1 eq) in Methanol: Water (1:1, 10 mL), NaOH (0.02 g, 5 eq) was added at room temperature. The above mixture was heated to 70° C. for 12 h. The progress of the reaction was monitored by TLC. After completion of reaction, the reaction mixture was concentrated and the resulting residue was washed with diethyl ether followed by treatment with water. The aqueous layer was neutralized to pH=7 using 1 N HCl at 0° C. The solid obtained was filtered, washed with water and dried under vacuum to afford the mixture of compound 4 and 4a.

Step 3: Synthesis of (9H-fluoren-9-yl)methyl (2-(4-((4-(2-hydroxy-2-methylpropyl)piperidin-1-yl)methyl)benzamido)phenyl)carbamate and (9H-fluoren-9-yl)methyl (2-(4-((4-(2-methylprop-1-en-1-yl)piperidin-1-yl)methyl)benzamido) phenyl)carbamate (6 and 6a): To a stirred solution of compound 4 and 4a (0.3 g, 1 eq) and compound 5 (0.339 g, 1 eq) in DMF (10 mL), DIPEA (0.45 mL, 2.5 eq) was added and stirred for 10 min. To this, HATU (0.586 g, 1.5 eq) was added and the reaction mixture was stirred at room temperature for overnight, the reaction progress was monitored by TLC and LCMS. After completion, the reaction mixture was portioned between ethyl acetate and water. The organic layers were separated, washed with water and brine, dried over Na₂SO₄ and evaporated to get the crude product which was purified by silica gel column chromatography to afford the desired compound 6 (0.1 g, 16.14%) and compound 6a (0.13 g, 21%).

Step 4: Synthesis of N-(2-aminophenyl)-4-((4-(2-hydroxy-2-methylpropyl)piperidin-1-yl)methyl)benzamide (Compound 181): A solution of compound 6 (0.1 g, 1 eq) in 20% piperidine in DMF (2 mL) was stirred at room temperature for 15 min. The progress of the reaction was monitored by TLC. After completion, the reaction mixture diluted with ice cold water. The solid obtained was filtered, washed with water; pentane and dried under vacuum to provide the desired compound.

¹H NMR (400 MHz, DMSO-d6) δ 9.61 (s, 1H), 8.16 (s, 1H), 7.92 (d, J=7.6 Hz, 2H), 7.40 (d, J=7.6 Hz, 2H), 7.14 (d, J=7.6 Hz, 1H), 6.95 (t, J=7.6 Hz, 1H), 6.76 (d, J=7.6, Hz, 1H), 6.58 (t, J=7.2 Hz, 1H), 3.51 (s, 2H), 2.76-2.72 (m, 2H), 1.96 (t, J=10.8 Hz, 2H), 1.70-1.68 (m, 2H), 1.43-1.40 (m, 1H), 1.30-1.09 (m, 4H), 1.07 (s, 6H); LCMS Calculated for C₂₃H₃₁N₃O₂ (free base): 381.24; Observed: 382.20 (M+1)⁺.

Step 5: Synthesis of N-(2-aminophenyl)-4-((4-(2-methylprop-1-en-1-yl)piperidin-1-yl)methyl)benzamide (Compound 472): A solution of compound 6a (0.13 g, 1 eq) in 20% piperidine in DMF (2 mL) was stirred at room temperature for 15 min. The progress of the reaction was monitored by TLC. After completion, the reaction mixture diluted with ice cold water. The solid obtained was filtered, washed with water; pentane and dried under vacuum to provide the desired compound.

¹H NMR (400 MHz, DMSO-d6) δ 9.61 (s, 1H), 7.92 (d, J=7.8 Hz, 2H), 7.40 (d, J=7.8 Hz, 2H), 7.15 (d, J=7.7 Hz, 1H), 6.95 (t, J=7.6 Hz, 1H), 6.76 (d, J=7.9 Hz, 1H), 6.58 (t, J=7.5 Hz, 1H), 4.95-4.87 (m, 1H), 4.71-4.50 (m, 1H), 3.50 (s, 2H), 2.81-2.71 (m, 2H), 2.13-2.00 (m, 1H), 2.02-1.86 (m, 2H), 1.67-1.44 (m, 7H), 1.30-1.24 (m, 1H), 1.17-1.02 (m, 1H); LCMS Calculated for C₂₃H₂₉N₃O (free base): 363.23; Observed: 364.20 (M+1)⁺.

### Synthetic Scheme for Compound 238 and Compound 241

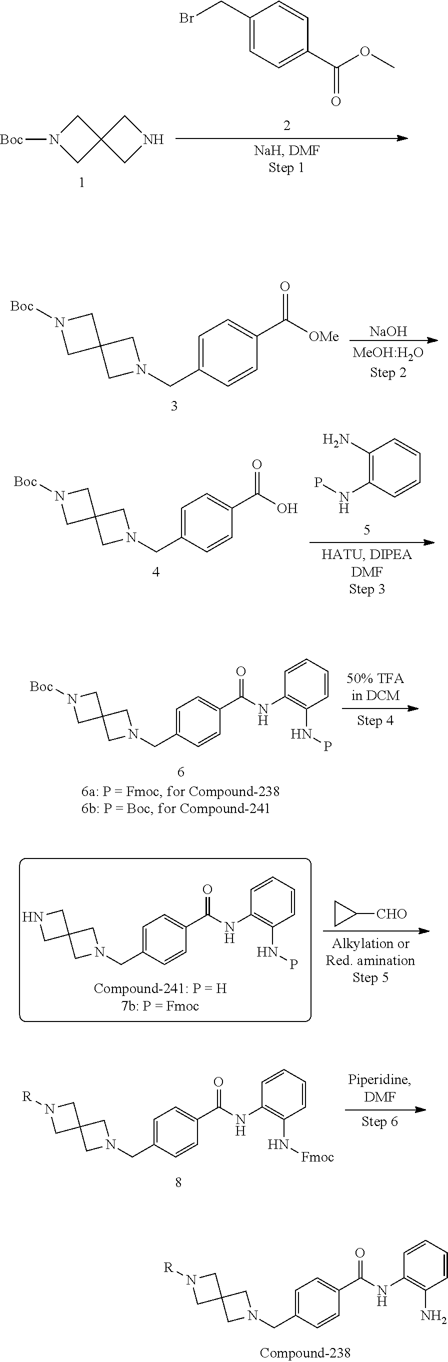

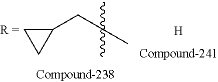

Step 1: Synthesis of tert-butyl 6-(4-(methoxycarbonyl)benzyl)-2,6-diazaspiro[3.3]heptane-2-carboxylate (3): To a stirred solution of compound 1 (1 g, 1 eq) in dry DMF (10 mL) at 0° C., NaH (60%, 0.123 g, 1.5 eq) was added slowly and stirred at same temperature for 30 min. To this solution, compound 2 (0.47 g, 1 eq) was added slowly. The resulting reaction mixture was stirred at room temperature for 1 h. The progress of the reaction was monitored by TLC. After completion, the reaction was quenched with water and extracted with ethyl acetate. The organic layers were separated, washed with water and dried over Na₂SO₄ and concentrated. The crude residue was purified by silica gel column chromatography to provide the desired compound.

Step 2: Synthesis of 4-((6-(tert-butoxycarbonyl)-2,6-diazaspiro[3.3]heptan-2-yl)methyl)benzoic acid (4): To stirred solution of ester compound 3 (0.53 g, 1 eq) in Methanol: Water (1:1, 8 mL), NaOH (0.091 g, 5eq) was added at room temperature. The above mixture was heated to 70° C. for 12 h. The progress of the reaction was monitored by TLC. After completion of reaction, the reaction mixture was concentrated and the resulting residue was washed with diethyl ether followed by treatment with water. The aqueous layer was neutralized to pH=7 using 1 N HCl at 0° C. The solid obtained was filtered, washed with water and dried under vacuum to afford the title compound 4.

Step 3: Synthesis of compound 6: To a stirred solution of acid compound 4 (1 eq) and corresponding amine 5 (1 eq) in DMF (10 mL), DIPEA (2.5 eq) was added and stirred for 10 min. To this, HATU (1.5 eq) was added and the reaction mixture was stirred at room temperature for overnight, the reaction progress was monitored by TLC and LCMS. After completion, the reaction mixture was portioned between ethyl acetate and water. The organic layers were separated, washed with water and brine, dried over Na₂SO₄ and evaporated to get the crude product which was purified by silica gel column chromatography to afford the desired compound.

| No | Structure |
|---|---|
| 1 | 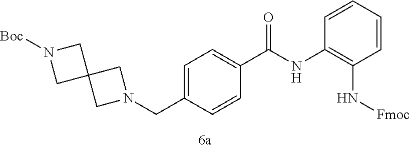 |
| 2 | 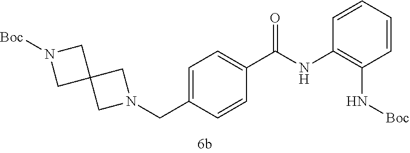 [SMILES: CC(C)(C)OC(=O)Nc1ccccc1NC(=O)c1ccc(CN2CC3(C2)CN(C(=O)OC(C)(C)C)C3)cc1] |

Step 4: Synthesis of compound 7b and Compound 241: A stirred solution of Compound 6a or 6b (0.01 g, 1 eq) in 50% TFA/DCM (0.5 mL) was stirred at room temperature for 15 min. The progress of the reaction was monitored by TLC. After completion, the reaction mixture was concentrated and the resulting residue was purified by basic resin (sp-carbonate) to afford the desired compound.

| No | Structure |
|---|---|
| 1 | 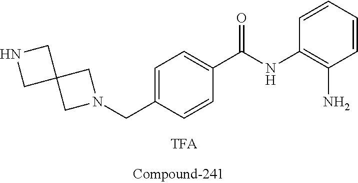 [SMILES: Nc1ccccc1NC(=O)c1ccc(CN2CC3(CNC3)C2)cc1] |
| 2 | 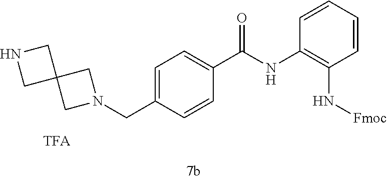 |

4-((2,6-diazaspiro[3.3]heptan-2-yl)methyl)-N-(2-aminophenyl)benzamide (Compound-241): Compound delivered as TFA salt.

¹H NMR (400 MHz, DMSO-d6) δ 9.63 (s, 1H), 8.69 (s, 1H), 7.93 (d, J=7.6 Hz, 2H), 7.36 (d, J=7.6 Hz, 2H), 7.15 (d, J=8.0 Hz, 1H), 6.97 (t, J=7.6 Hz, 1H), 6.78 (d, J=8.0 Hz, 1H), 6.60 (t, J=7.2 Hz, 1H), 4.88 (s, 2H), 4.07-4.05 (m, 4H), 3.67-3.62 (m, 2H), 3.43-3.32 (m, 4H); LCMS Calculated for C₁₉H₂₂N₄O (free base): 322.18; Observed: 322.85 (M+1)⁺.

Step 5: Synthesis of compound (9H-fluoren-9-yl)methyl (2-(4-((6-(cyclopropylmethyl)-2,6-diazaspiro[3.3]heptan-2-yl)methyl)benzamido)phenyl)carbamate (8): To a stirred solution of amine compound 7b (1 eq) and corresponding aldehyde (1.2 eq) in DCM, acetic acid (6 eq) was added and stirred at room temperature for 30 min. To this, sodium triacetoxyborohydride (STAB) (3 eq) was added at room temperature. The resulting reaction mixture was stirred at room temperature for overnight; the reaction progress was monitored by TLC and LCMS. After completion, the reaction mixture was quenched with sat. NaHCO₃ solution and extracted with DCM. The organic layers were separated, washed with water and brine, dried over Na₂SO₄ and evaporated to get the crude product which was purified by silica gel column chromatography to afford the desired compound.

Step 6: Synthesis of N-(2-aminophenyl)-4-((6-(cyclopropylmethyl)-2,6-diazaspiro[3.3]heptan-2-yl)methyl)benzamide (Compound-238): A solution of compound 8 (0.04 g, 1 eq) in 20% piperidine in DMF (1 mL) was stirred at room temperature for 15 min. The progress of the reaction was monitored by TLC. After completion, the reaction mixture diluted with ice cold water and extracted with 10% MeOH/DCM. The organic layers were separated, washed with sat. NaHCO₃ solution and brine, dried over Na₂SO₄ and evaporated to get the crude product which was purified by silica gel column chromatography to afford the desired compound.

¹H NMR (400 MHz, DMSO-d6) δ9.61 (s, 1H), 7.91 (d, J=6.4 Hz, 2H), 7.35 (d, J=6.4 Hz, 2H),7.15 (d, J=6.0 Hz, 1H), 6.97-6.95 (m, 1H), 6.78 (d, J=7.6 Hz, 1H), 6.59-6.57 (m, 1H), 4.88 (s, 2H), 3.58-3.56 (m, 2H), 3.24-3.21 (m, 8H), 2.22-2.20 (m, 2H), 1.24-1.22 (m, 1H), 0.39-0.31 (m, 2H), 0.05-0.04 (m, 2H); LCMS Calculated for C₂₃H₂₈N₄O (free base): 376.23; Observed: 376.95 (M+1)⁺.

### Synthetic Scheme for Compound 176

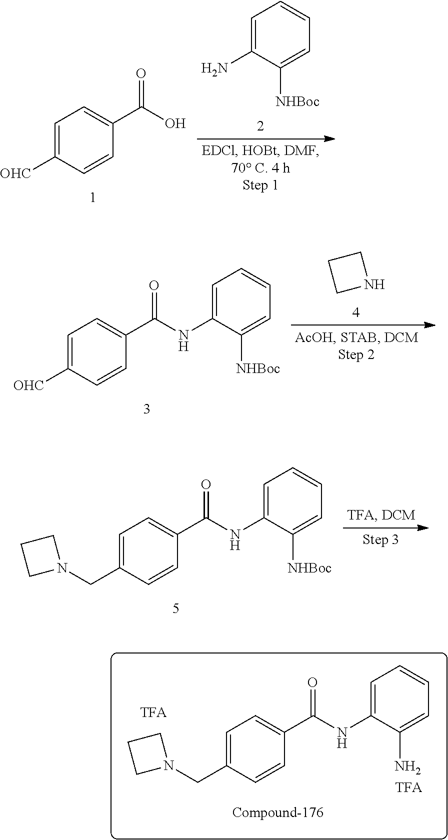

Step 1: Synthesis of tert-butyl (2-(4-formylbenzamido)phenyl)carbamate (3): To a stirred solution of compound 1 (0.5 g, 1 eq) and compound 2 (0.693 g, 1.2 eq) in DMF (5 mL), HOBt (0.45 g, 1 eq) and EDCl HCl (0.64 g, 1 eq) were added. The resulting reaction mixture was stirred at 70° C. for 4h; the reaction progress was monitored by TLC. After completion, the reaction mixture was quenched with water and extracted with ethyl acetate. The organic layers were separated, washed with sat. NaHCO₃ solution and brine; dried over Na₂SO₄ and evaporated to get the crude product which was purified by silica gel column chromatography to afford the desired compound 3.

Step 2: Synthesis of tert-butyl (2-(4-(azetidin-1-ylmethyl)benzamido) phenyl)carbamate (5) To a stirred solution of compound 3 (0.3 g, 1 eq) and compound 4 (0.06 g, 1.2 eq) in DCM (6 mL), acetic acid (0.317 g, 6 eq) was added and stirred at room temperature for 30 min. To this, sodium triacetoxyborohydride (STAB) (0.561 g, 3 eq) was added at room temperature. The resulting reaction mixture was stirred at room temperature for overnight; the reaction progress was monitored by TLC and LCMS. After completion, the reaction mixture was quenched with sat. NaHCO₃ solution and extracted with DCM. The organic layers were separated, washed with water and brine, dried over Na₂SO₄ and evaporated to get the crude product which was purified by silica gel column chromatography to afford the desired compound 5.

Step 3: Synthesis of N-(2-aminophenyl)-4-(azetidin-1-ylmethyl)benzamide (Compound 176): A mixture of compound 5 (0.07 g, 1 eq) and 50% TFA/DCM (2 mL) was stirred at room temperature for 15 min. The progress of the reaction was monitored by TLC. After completion, the reaction mixture was concentrated and the resulting residue was triturated with n-pentane, diethyl ether and dried under vacuum to give the desired compound as TFA salt.

¹H NMR (400 MHz, DMSO-d6) δ 10.28 (s, 1H), 9.93 (s, 1H), 8.03 (d, J=7.6 Hz, 2H), 7.58 (d, J=8.0 Hz, 2H), 7.23 (d, J=7.6 Hz, 1H), 7.08 (t, J=7.2 Hz, 1H), 6.95 (d, J=8.0 Hz, 1H), 6.81 (t, J=7.2 Hz, 1H), 4.44-4.42 (m, 2H), 4.11-3.99 (m, 4H), 2.46-2.25 (m, 2H); LCMS Calculated for C₁₇H₁₉N₃O (free base): 281.15; Observed: 282.05 (M+1)⁺.

### Synthetic Scheme for Compound 171, Compound 172, Compound 174 and Compound 175

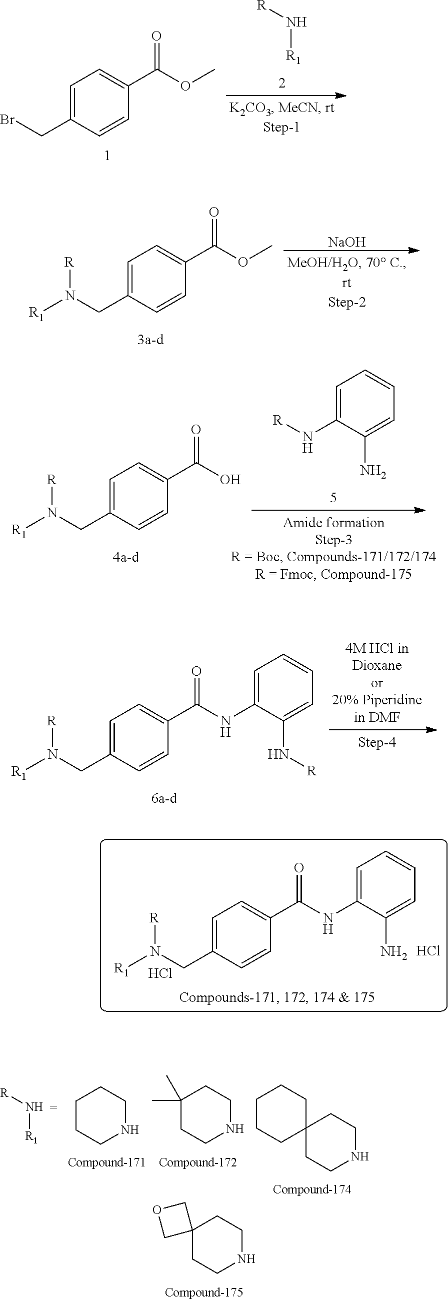

Step 1: Synthesis of Compound 3: To a stirred solution of respective amine 2 (1 eq) in ACN at 0° C., potassium carbonate (3 eq) was added. To this, compound 1 (1 eq) was added and the reaction mixture was stirred at room temperature for 16 h. The progress of reaction was monitored by TLC. After completion, the reaction mixture was diluted with water and extracted with ethyl acetate. The combined organic extracts were washed with water, brine, dried over anhydrous Na₂SO₄, filtered and concentrated under reduced pressure to afford the crude product which was purified by silica gel column chromatography to afford the desired compound 3a-d.

| No | Structure |
|---|---|
| 1 | 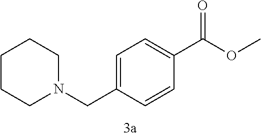 [SMILES: COC(=O)c1ccc(CN2CCCCC2)cc1] |
| 2 | 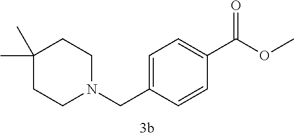 |
| 3 | 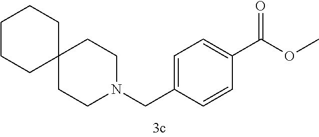 [SMILES: COC(=O)c1ccc(CN2CCC3(CCCCC3)CC2)cc1] |
| 4 | 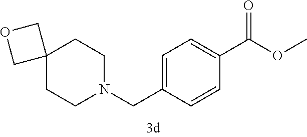 [SMILES: COC(=O)c1ccc(CN2CCC3(CC2)COC3)cc1] |

Step 2: Synthesis of Compound 4: To stirred solution of corresponding ester compound 3a-d (1 eq) in Methanol: Water (1:1), NaOH (1.5 eq) was added at room temperature. The above mixture was heated to 70° C. for 12 h. The progress of the reaction was monitored by TLC. After completion of reaction, the reaction mixture was concentrated and the resulting residue was washed with diethyl ether followed by treatment with water. The aqueous layer was neutralized to pH=7 using 1 N HCl at 0° C. The solid obtained was filtered, washed with water and dried under vacuum to afford the mixture of compound 4a-d.

| No | Structure |
|---|---|
| 1 | 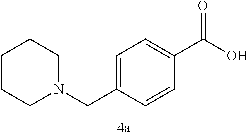 [SMILES: O=C(O)c1ccc(CN2CCCCC2)cc1] |
| 2 | 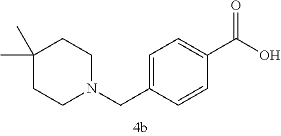 [SMILES: CC1(C)CCN(Cc2ccc(C(=O)O)cc2)CC1] |
| 3 | 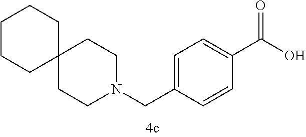 [SMILES: O=C(O)c1ccc(CN2CCC3(CCCCC3)CC2)cc1] |
| 4 | 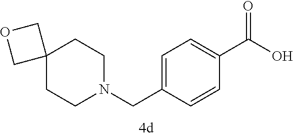 [SMILES: O=C(O)c1ccc(CN2CCC3(CC2)COC3)cc1] |

Step 3: Synthesis of Compound 6a-c: To a stirred solution of respective acid compound 4a-c (1 eq) and respective amine 5 (1.1 eq) in ACN, pyridine (5 eq) and HATU (1.5 eq) was added at room temperature. After stirring the reaction mixture at 80° C. for overnight, the reaction progress was monitored by TLC and LCMS. After completion, the reaction mixture was concentrated and resulting residue was portioned between water and ethyl acetate. The organic layers were separated, washed with water and 1% HCl to remove traces of pyridine, dried over Na₂SO₄ and concentrated. The crude residue was purified by silica gel column chromatography to provide the desired compound 6a-c.

Step 3: Synthesis of Compound 6d: To a stirred solution of compound 4d (1 eq) and respective amine 5 (1 eq) in DMF, DIPEA (2.5 eq) was added and stirred for 10 min. To this, HATU (1.5 eq) was added and the reaction mixture was stirred at room temperature for overnight, the reaction progress was monitored by TLC and LCMS. After completion, the reaction mixture was portioned between ethyl acetate and water. The organic layers were separated, washed with water and brine, dried over Na₂SO₄ and evaporated to get the crude product which was purified by silica gel column chromatography to afford the desired compound 6d.

| No | Structure |
|---|---|
| 1 | 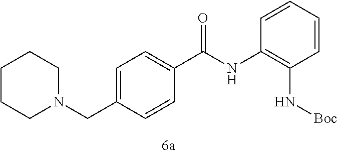 [SMILES: CC(C)(C)OC(=O)Nc1ccccc1NC(=O)c1ccc(CN2CCCCC2)cc1] |
| 2 | 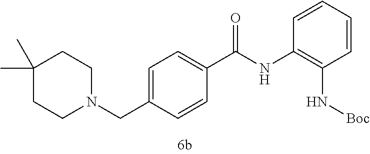 [SMILES: CC1(C)CCN(Cc2ccc(C(=O)Nc3ccccc3NC(=O)OC(C)(C)C)cc2)CC1] |
| 3 | 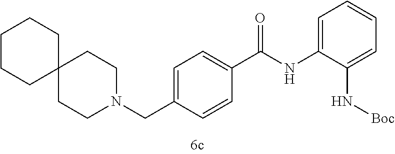 [SMILES: CC(C)(C)OC(=O)Nc1ccccc1NC(=O)c1ccc(CN2CCC3(CCCCC3)CC2)cc1] |
| 4 | 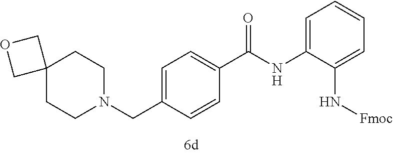 |

Step 4: Synthesis of N-(2-aminophenyl)-4-(piperidin-1-ylmethyl)benzamide (Compound 171): To a stirred solution of Boc compound 6a (1 eq) in 1,4-dioxane (5 vol), 4 M HCl in dioxane (5 vol) was added. The resulting reaction mixture was stirred at room temperature for 1 h. The progress of the reaction was monitored by TLC. After completion, the reaction mixture was concentrated and the resulting residue was triturated with n-pentane and dried under vacuum to give desired compound as HCl salt.

¹H NMR (400 MHz, DMSO-d6) δ 10.28 (s, 2H), 8.13 (d, J=7.6 Hz, 2H), 7.74 (d, J=8.0 Hz, 2H), 7.41 (d, J=7.6 Hz, 1H), 7.30-7.22 (m, 3H), 4.35 (d, J=4.4 Hz, 2H), 3.30-3.27 (m, 2H), 2.88-2.85 (m, 2H), 1.85-1.65 (m, 5H), 1.37-1.35 (m, 1H); LCMS Calculated for C₁₉H₂₃N₃O (free base): 309.18; Observed: 309.90(M+1)⁺.

Step 4: Synthesis of N-(2-aminophenyl)-4-((4,4-dimethylpiperidin-1-yl)methyl)benzamide (Compound 172): To a stirred solution of Boc compound 6b (1 eq) in 1,4-dioxane(5 vol), 4 M HCl in dioxane (5 vol), was added. The resulting reaction mixture was stirred at room temperature for 1 h. The progress of the reaction was monitored by TLC. After completion, the reaction mixture was concentrated and the resulting residue was triturated with n-pentane and dried under vacuum to give desired compound as HCl salt.

¹H NMR (400 MHz, DMSO-d6) δ 10.32 (s, 1H), 10.10 (bs, 1H), 8.13 (d, J=8.0 Hz, 2H), 7.74 (d, J=7.6 Hz, 2H), 7.42 (d, J=8.0 Hz, 1H), 7.24-7.16 (m, 3H), 4.40 (d, J=5.2 Hz, 2H), 3.24-3.00 (m, 4H), 1.71-1.65 (m, 2H), 1.52-1.48 (m, 2H), 1.01 (s, 3H), 0.96 (s, 3H); LCMS Calculated for C₂₁H₂₇N₃O (free base): 337.22; Observed: 337.88 (M+1)⁺.

Step 4: Synthesis of 4-((3-azaspiro[5.5]undecan-3-yl)methyl)-N-(2-aminophenyl)benzamide (Compound 174): To a stirred solution of Boc compound 6c (1 eq) in 1,4-dioxane (5 vol), 4 M HCl in dioxane (5 vol), was added. The resulting reaction mixture was stirred at room temperature for 1 h. The progress of the reaction was monitored by TLC. After completion, the reaction mixture was concentrated and the resulting residue was triturated with n-pentane and dried under vacuum to give desired compound as HCl salt.

¹H NMR (400 MHz, DMSO-d6) δ 10.58 (s, 1H), 10.42 (s, 1H), 8.14 (d, J=8.0 Hz, 2H), 7.76 (d, J=8.0 Hz, 2H), 7.48 (d, J=7.7 Hz, 1H), 7.34-7.15 (m, 3H), 4.37 (d, J=5.2 Hz, 2H), 3.13-2.96 (m, 4H), 1.78-1.68 (m, 2H), 1.65-1.55 (m, 2H), 1.49-1.47 (m, 2H), 1.38-1.34 (m, 6H), 1.22-1.19 (m, 2H); 90.28%; LCMS Calculated for C₂₄H₃₁N₃O (free base): 377.25; Observed: 378.01 (M+1)⁺.

Step 4: Synthesis of 4-((2-oxa-7-azaspiro[3.5]nonan-7-yl)methyl)-N-(2-aminophenyl)benzamide (Compound 175): A mixture of compound 6d (10 mg, 1 eq) and 20% piperidine in DMF (0.5 mL) was stirred at room temperature for 15 min. The progress of the reaction was monitored by TLC. After completion, the reaction mixture diluted with ice cold water. The solid obtained was filtered, washed with water; pentane and dried under vacuum to provide the desired compound.

¹H NMR (400 MHz, DMSO-d6) δ 9.61 (s, 1H), 7.93 (d, J=8.0 Hz, 2H), 7.40 (d, J=8.0 Hz, 2H), 7.16 (d, J=7.6 Hz, 1H), 7.01-6.92 (m, 1H), 6.77 (d, J=7.2 Hz, 1H), 6.59 (t, J=7.2 Hz, 1H), 4.88 (s, 2H), 4.30-4.25 (m, 4H), 3.48 (s, 2H), 2.27-2.25 (m, 4H), 1.79-1.75 (m, 4H); LCMS Calculated for C₂₁H₂₅N₃O₂ (free base): 351.19; Observed: 351.80 (M+1)⁺.

### Synthetic Scheme for Compound 354

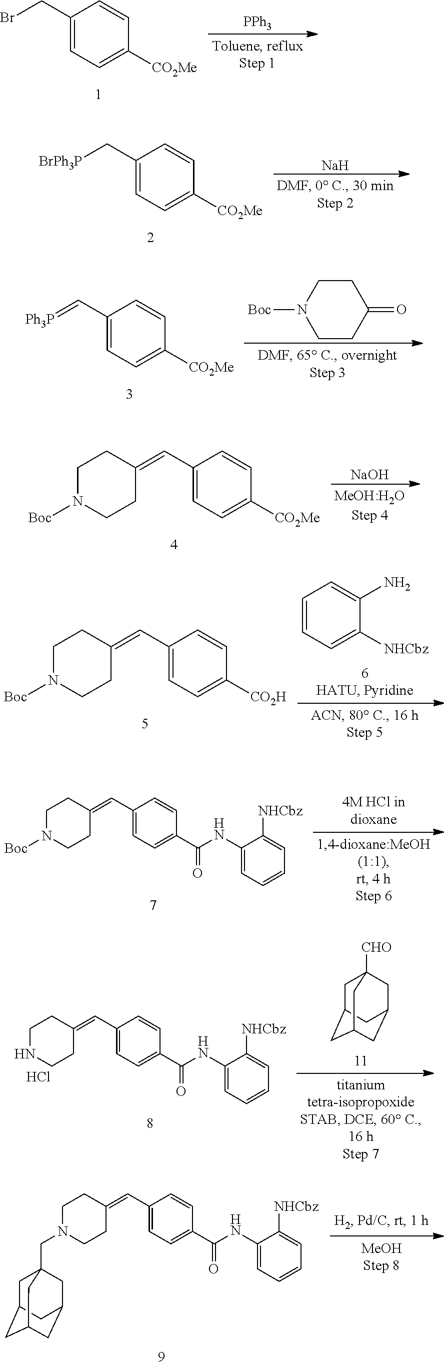

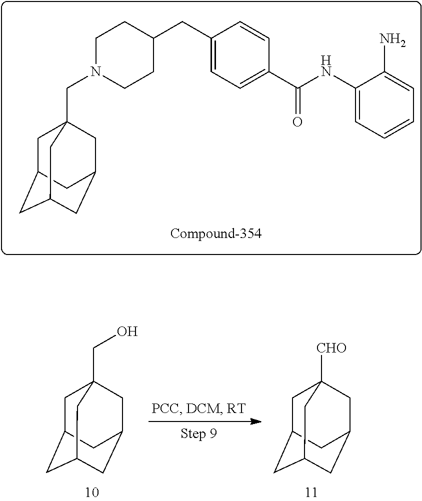 [SMILES: Nc1ccccc1NC(=O)c1ccc(CC2CCN(CC34CC5CC(CC(C5)C3)C4)CC2)cc1 ; OCC12CC3CC(CC(C3)C1)C2 ; O=CC12CC3CC(CC(C3)C1)C2]

Step 1: Synthesis of methyl 4-((bromotriphenyl-l5-phosphanyl)methyl)benzoate (2): To a stirred solution of compound 1 (50 g, 1 eq) in toluene (500 mL), triphenyl phosphine (55.5 g, 1 eq) was added and the reaction mixture was heated at reflux for 17 h. After 17 h, the reaction mixture was allowed to cool to room temperature, the precipitate was filtered, washed with toluene followed by hexane and dried under vacuum to afford the title compound 2.

Steps 2 and 3: Synthesis of tert-butyl 4-(4-(methoxycarbonyl)benzylidene) piperidine-1-carboxylate (4): To a stirred solution of compound 2 (100 g, 1 eq) in DMF (500 mL) at 0° C., NaH (60%, 207 g, 1.1 eq) was added slowly and stirred at same temperature for 30 min. To this solution, tert-butyl 4-oxopiperidine-1-carboxylate (10.75 g, 1.1 eq) was added at 0° C. The resulting reaction mixture was stirred at 65° C. for overnight. The progress of the reaction was monitored by TLC. After completion, the reaction was quenched with water and extracted with ethyl acetate. The organic layers were separated, washed with water and dried over Na₂SO₄ and concentrated. The crude residue was purified by silica gel column chromatography to provide the title compound 4.

Step 4: Synthesis of 4-((1-(tert-butoxycarbonyl)piperidin-4-ylidene)methyl)benzoic acid (5): To stirred solution of ester compound 4 (1 g, 1 eq) in Methanol: Water (1:1, 20 mL), NaOH (0.181 g, 1.5 eq) was added at room temperature. The above mixture was heated to 70° C. for 12 h. The progress of the reaction was monitored by TLC. After completion of reaction, the reaction mixture was concentrated and the resulting residue was washed with diethyl ether followed by treatment with water. The aqueous layer was neutralized to pH=7 using 1 N HCl at 0° C. The solid obtained was filtered, washed with water and dried under vacuum to provide the title compound 5.

Step 5: Synthesis of tert-butyl 4-(4-((2-(((benzyloxy)carbonyl)amino)phenyl) carbamoyl)benzylidene)piperidine-1-carboxylate (7): To a stirred solution of acid compound 5 (0.85 g, 1 eq) and amine 6 (0.716 g, 1.1 eq) in ACN (16 mL), pyridine (1.05 mL, 5 eq) and HATU (1.53 g, 1.5 eq) was added at room temperature. The reaction mixture was stirred at 80° C. for 16 h; the reaction progress was monitored by TLC. After completion, the reaction mixture was concentrated and resulting residue was diluted with water and extracted with ethyl acetate. The organic layers were separated, washed with water and 1% HCl to remove traces of pyridine, dried over Na₂SO₄ and concentrated. The crude residue was purified by silica gel column chromatography to provide the title compound 7.

Step 6: Synthesis of benzyl (2-(4-(piperidin-4-ylidenemethyl)benzamido) phenyl)carbamate hydrochloride (8): To a stirred solution of Boc compound 7 (1 g, 1 eq) in 1,4-dioxane (10 mL), 4 M HCl in dioxane (4 mL) was added and the reaction was stirred at room temperature for 1 h. After completion, the reaction mixture was concentrated and the resulting residue was triturated with diethyl ether; acetonitrile and dried under vacuum to give the title compound 8 as HCl salt.

Step 9: Synthesis of (3r,5r,7r)-adamantane-1-carbaldehyde (11): To a stirred solution of compound 10 (1 g, 1 eq) in DCM (10 mL) at 0° C., PCC (1.42 g, 1.1 eq) was added portion wise. The resulting reaction mass was stirred at room temperature for 2 h. The progress of the reaction was monitored by TLC. After completion, the resulting mixture was filtered over a pad of celite. The filtrate was washed with water; the organic layer was separated; dried over anhydrous sodium sulfate and concentrated under reduced pressure to provide the title compound 11.

Step 7: Synthesis of benzyl (2-(4-((1-(((3r,5r,7r)-adamantan-1-yl)methyl) piperidin-4-ylidene)methyl)benzamido)phenyl)carbamate (9): To a stirred solution of compound 8 (0.3 g, 1 eq) and compound 11 (0.155 g, 1.5 eq) in DCE (8 mL) titanium tetra-isopropoxide (Ti(OiPr)₄)(1.04 g, 6 eq) was added at room temperature. After 5 min. STAB (0.298 g, 3 eq) was added and the mixture was heated at 60° C. for 16 h. The progress of the reaction was monitored by TLC and LCMS. After completion, the reaction mixture was diluted with DCM and the resulting mixture was filtered over a pad of celite. The filtrate was concentrated and the resulting residue was purified by silica gel column chromatography to provide the title compound 9.

Step 8: Synthesis of 4-((1-(((3r,5r,7r)-adamantan-1-yl)methyl)piperidin-4-yl)methyl)-N-(2-aminophenyl)benzamide (Compound 354): To a stirred solution of compound 9 (0.11 g, 1 eq) in methanol (3 mL), 10% Pd/C (50 mg) was added and the reaction mixture was stirred under hydrogen atmosphere (balloon pressure) at room temperature for 1 h. The progress of the reaction was monitored by TLC. After completion, the reaction mixture was filtered through a pad of celite, the filtrate was evaporated under reduced pressure and the resulting residue was purified by prep. HPLC to afford the title compound.

¹H NMR (400 MHz, DMSO-d6) δ 9.58 (s, 1H), 7.89 (d, J=8.0 Hz, 2H), 7.28 (d, J=7.6 Hz, 2H), 7.15 (d, J=7.6 Hz, 1H), 6.96 (t, J=8.0 Hz, 1H), 6.77 (d, J=7.2 Hz, 1H), 6.59 (t, J=7.2 Hz, 1H), 4.88 (s, 2H), 2.68-2.65 (m, 2H), 2.58-2.56 (m, 2H), 2.13-2.02 (m, 2H), 1.90-1.83 (m, 5H), 1.67-1.57 (m, 6H), 1.50-1.40 (m, 9H), 1.27-1.21 (m, 2H); LCMS Calculated for C₃₀H₃₉N₃O (free base): 457.31; Observed: 458.43 (M+1)⁺.

### Synthetic Scheme for Compound 169

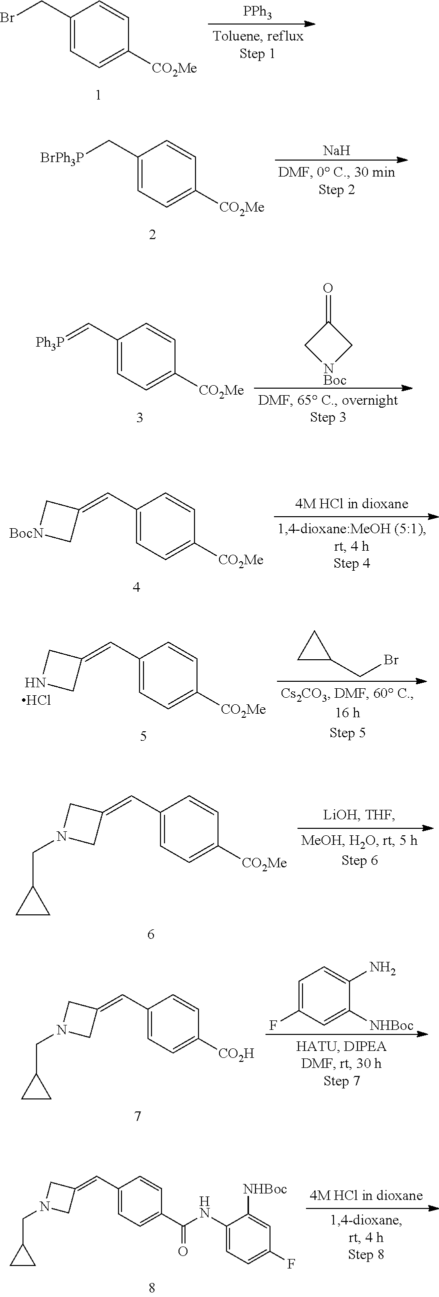

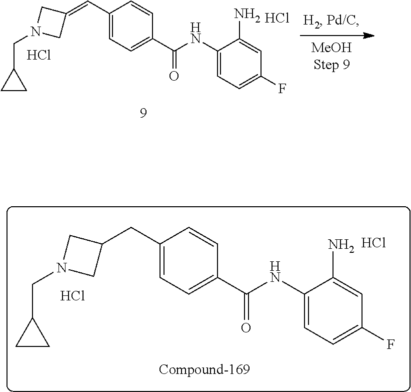 [SMILES: Nc1cc(F)ccc1NC(=O)c1ccc(C=C2CN(CC3CC3)C2)cc1 ; Cl ; Cl ; Nc1cc(F)ccc1NC(=O)c1ccc(CC2CN(CC3CC3)C2)cc1 ; Cl ; Cl]

Step 1: Synthesis of methyl 4-((bromotriphenyl-l5-phosphanyl)methyl)benzoate (2): To a stirred solution of compound 1 (50 g, 1 eq) in toluene (500 mL), triphenyl phosphine (55.5 g, 1 eq) was added and the reaction mixture was heated at reflux for 17 h. After 17 h, the reaction mixture was allowed to cool to room temperature, the precipitate was filtered, washed with toluene followed by hexane and dried under vacuum to afford the title compound 2.

Step 2 and 3: Synthesis of tert-butyl 3-(4-(methoxycarbonyl) benzylidene)azetidine-1-carboxylate (4): To a stirred solution of compound 2 (70 g, 1 eq) in dry DMF (350 mL) at 0° C., NaH (60% in mineral oil, 7.9 g, 1.4 eq) was added slowly and stirred at same temperature for 30 min. To this solution, tert-butyl 3-oxoazetidine-1-carboxylate (24.4 g, 1 eq) was added at 0° C. The resulting reaction mixture was stirred at 65° C. for overnight. The progress of reaction was monitored by TLC. After completion, the reaction mixture was cooled to 0° C. and quenched with sat. NH₄C; solution, the precipitate was filtered. The residue was taken in acetonitrile, stirred for 10 min and again filtered. The solid washed with acetonitrile. The crude product was purified by silica gel column chromatography to afford the compound 4.

Step 4: Synthesis of methyl 4-(azetidin-3-ylidenemethyl)benzoate hydrochloride (5) To a stirred solution of Boc compound 4 (26 g, 1 eq) in 1,4-dioxane: methanol (30 mL: 20 mL) mixture , 4 M HCl in dioxane (150 mL) was added and the reaction was stirred at room temperature for 4 h. After completion, the reaction mixture was concentrated and the resulting residue was triturated with n-pentane, diethyl ether and dried under vacuum to give the desired compound 5 as HCl salt.

Step 5: Synthesis of methyl 4-((1-(cyclopropylmethyl)azetidin-3-ylidene)methyl)benzoate (6): To a stirred solution of compound 5 (1.5 g, 1 eq) in DMF (20 mL), cesium carbonate (5.09 g, 2.5 eq) was added and stirred at room temperature for 10 min. To this solution, (bromomethyl)cyclopropane (0.847 g, 1 eq) was added. The resulting reaction mixture was stirred at 60° C. for 16 h. The progress of the reaction was monitored by TLC. After completion, the reaction was quenched with ice cold water and extracted with ethyl acetate. The organic layers were separated, washed with water and dried over Na₂SO₄ and concentrated. The crude residue was purified by silica gel column chromatography to provide the desired compound 6.

Step 6: Synthesis of 4-((1-(cyclopropylmethyl)azetidin-3-ylidene)methyl)benzoic acid (7): To stirred solution of ester compound 6 (0.53 g, 1 eq) in MeOH:THF (1:3) mixture, aqueous LiOH (0.258 g, 3 eq, dissolved in 0.75 mL of water) was added. The reaction mixture was stirred at room temperature for 5 h. The progress of the reaction was monitored by TLC. After completion, the reaction mixture was concentrated and the resulting residue was taken in water and acidified to pH=6 using 2 N HCl. The solid obtained was filtered, washed with water and dried under vacuum to provide the desired compound 7.

Step 7: Synthesis of tert-butyl (2-(4-((1-(cyclopropylmethyl)azetidin-3-ylidene)methyl)benzamido)-5-fluorophenyl)carbamate (8): To a stirred solution of compound 7 (0.4 g, 1 eq) and tert-butyl (2-amino-5-f luorophenyl)carbamate (0.407 g, 1.1 eq) in DMF (10 mL), DIPEA (0.845 g, 4 eq) was added and stirred for 10 min. To this, HATU (0.116 g, 1.5 eq) was added and the reaction mixture was stirred at room temperature for overnight, the reaction progress was monitored by TLC. After completion, the reaction mixture was diluted with water and extracted with ethyl acetate. The organic layers were separated, washed with water and brine, dried over Na₂SO₄ and evaporated to get the crude product which was purified by silica gel column chromatography to afford the desired compound 8.

Step 8: Synthesis of N-(2-amino-4-fluorophenyl)-4-((1-(cyclopropylmethyl)azetidin-3-ylidene)methyl)benzamide dihydrochloride (9): To a stirred solution of Boc compound 8 (0.18 g, 1 eq) in 1,4-dioxane (1 mL), 4 M HCl in dioxane (3 mL) and the reaction was stirred at room temperature for 4 h. After completion, the reaction mixture was concentrated and the resulting residue was triturated with diethyl ether, acetonitrile and dried under vacuum to give the desired compound 9 as HCl salt.

Step 9: Synthesis of N-(2-amino-4-fluorophenyl)-4-((1-(cyclopropylmethyl) azetidin-3-yl)methyl)benzamide (Compound-169): To a stirred solution of 9 (0.14 g, 1 eq) in methanol (10 mL), 10% Pd/C (30 mg) was added and the reaction mixture was stirred under hydrogen atmosphere (balloon pressure) at room temperature for 2 h. The progress of the reaction was monitored by TLC. After completion, the reaction mixture was filtered through a pad of celite, the filtrate was evaporated under reduced pressure and the resulting residue was triturated with diethyl ether and acetonitrile and then dried under vacuum to afford the title compound.

¹H NMR (400 MHz, DMSO-d6) δ 9.52 (s, 1H), 7.89 (d, J=8.0 Hz, 2H), 7.29 (d, J=7.6 Hz, 2H), 7.11-7.07 (m, 1H), 6.55-6.51 (m, 1H), 6.37-6.33 (m, 1H), 5.22 (s, 2H), 3.27 (t, J=6.8 Hz, 2H), 2.88-2.85 (m, 2H), 2.80 (t, J=6.4 Hz, 2H), 2.64-2.61 (m, 1H), 2.21-2.20 (m, 2H), 0.68-0.66 (m, 1H), 0.40-0.29 (m, 2H), 0.06-0.04 (m, 2H); LCMS Calculated for C₂₁H₂₄FN₃O (free base): 353.19; Observed: 353.90 (M+1)⁺.

### Synthetic Scheme for Compound 161, Compound 162, Compound 163

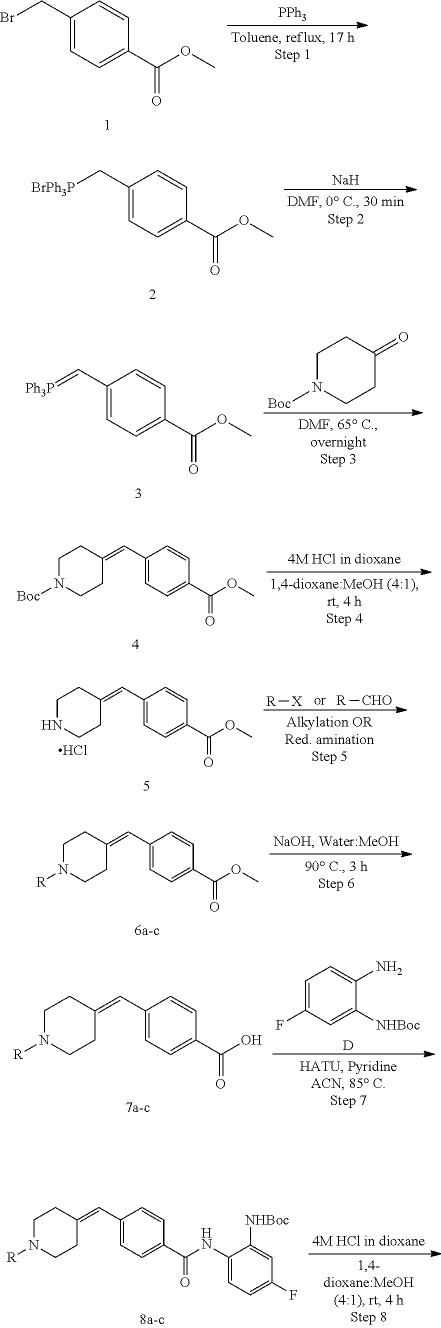

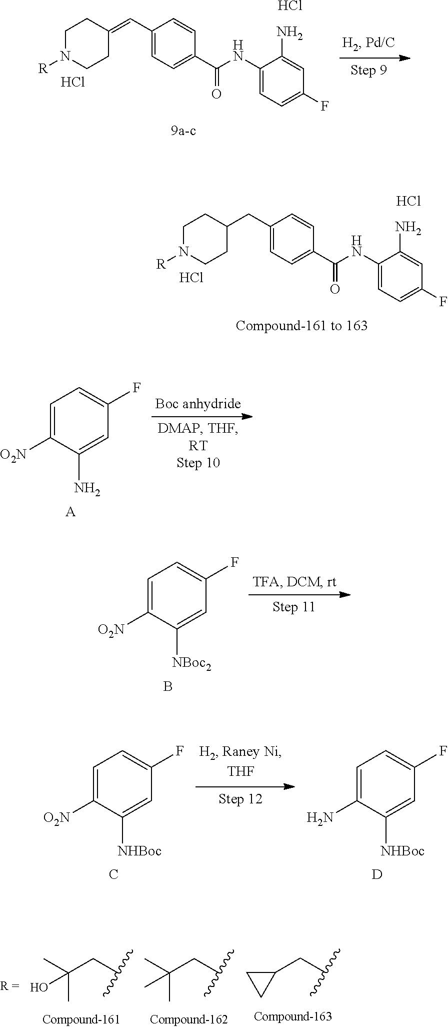

Step 1: Synthesis of methyl 4-((bromotriphenyl-l5-phosphanyl)methyl)benzoate (2): To a stirred solution of compound 1 (50 g, 1 eq) in toluene (500 mL), triphenyl phosphine (55.5 g, 1 eq) was added and the reaction mixture was heated at reflux for 17 h. After 17 h, the reaction mixture was allowed to cool to room temperature, the precipitate was filtered, washed with toluene followed by hexane and dried under vacuum to afford the title compound 2.

Steps 2 and 3: Synthesis of tert-butyl 4-(4-(methoxycarbonyl) benzylidene)piperidine-1-carboxylate (4): To a stirred solution of compound 2 (100 g, 1 eq) in DMF (500 mL) at 0° C., NaH (60%, 207 g, 1.1 eq) was added slowly and stirred at same temperature for 30 min. To this solution, tert-butyl 4-oxopiperidine-1-carboxylate (10.75 g, 1.1 eq) was added at 0° C. The resulting reaction mixture was stirred at 65° C. for overnight. The progress of the reaction was monitored by TLC. After completion, the reaction was quenched with water and extracted with ethyl acetate. The organic layers were separated, washed with water and dried over Na₂SO₄ and concentrated. The crude residue was purified by silica gel column chromatography to provide the title compound 4.

Step 4: Synthesis of methyl 4-(piperidin-4-ylidenemethyl)benzoate hydrochloride (5): To a stirred solution of Boc compound 4 (11 g, 1 eq) in 1,4-dioxane:methanol (4:1, 200 mL) mixture, 4 M HCl in dioxane (120 mL) was added and the reaction was stirred at room temperature for 3 h. After completion, the reaction mixture was concentrated and the resulting residue was triturated with diethyl ether and dried under vacuum to give the title compound 5 as HCl salt.

Step 5: Synthesis of compound 6a: To a solution of compound 5 (0.5 g, 1 eq) in ethanol (10 mL), TEA (0.8 mL, 3 eq) and 2,2-dimethyloxirane (0.203 g, 1.5 eq) were added at room temperature and the reaction mixture was heated at 60° C. for 12 h. The progress of reaction was monitored by TLC. After completion, the reaction mixture was allowed to cool, concentrated to give a crude compound which was purified by silica gel column chromatography.

Step 5: Synthesis of compounds 6b and 6c: To a stirred solution of compound 5 (3.5 g, 1 eq) in DMF (35 mL), cesium carbonate (10.7 g, 2.5 eq) was added and stirred at room temperature for 10 min. To this solution, (bromomethyl)cyclopropane (1.6 mL, 1.2 eq) was added. The resulting reaction mixture was stirred at 70° C. for 16 h. The progress of the reaction was monitored by TLC. After completion, the reaction was quenched with water and extracted with ethyl acetate. The organic layers were separated, washed with water and dried over Na₂SO₄ and concentrated. The crude residue was purified by silica gel column chromatography to provide the desired compound.

| No | Structure |
|---|---|
| 1 | 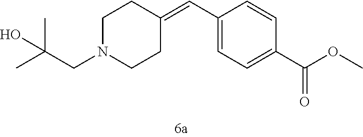 [SMILES: COC(=O)c1ccc(C=C2CCN(CC(C)(C)O)CC2)cc1] |
| 2 | 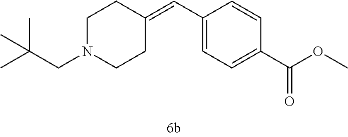 [SMILES: COC(=O)c1ccc(C=C2CCN(CC(C)(C)C)CC2)cc1] |
| 3 | 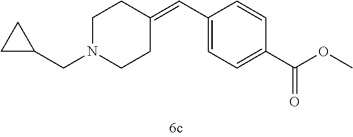 [SMILES: COC(=O)c1ccc(C=C2CCN(CC3CC3)CC2)cc1] |

Step 6: Synthesis of compound 7a-c: To stirred solution of ester compound 6 (1 eq) in Methanol: Water (1:1), NaOH (1.5 eq) was added at room temperature. The above mixture was heated to 70° C. for 12 h. The progress of the reaction was monitored by TLC. After completion of reaction, the reaction mixture was concentrated and the resulting residue was washed with diethyl ether followed by treatment with water. The aqueous layer was neutralized to pH=7 using 1 N HCl at 0° C. The solid obtained was filtered, washed with water and dried under vacuum to provide the desired compound.

| No | Structure |
|---|---|
| 1 | 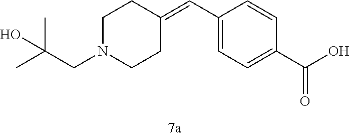 [SMILES: CC(C)(O)CN1CCC(=Cc2ccc(C(=O)O)cc2)CC1] |
| 2 | 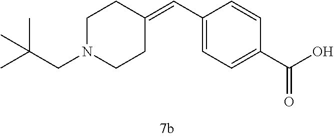 [SMILES: CC(C)(C)CN1CCC(=Cc2ccc(C(=O)O)cc2)CC1] |
| 3 | 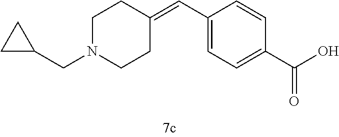 [SMILES: O=C(O)c1ccc(C=C2CCN(CC3CC3)CC2)cc1] |

Step 7: Synthesis of compound 8a-c: To a stirred solution of acid compound 7 (g, 1 eq) and tert-butyl (2-amino-5-fluorophenyl)carbamate (1.1 eq) in ACN, pyridine (5 eq) and HATU (1.5 eq) was added at room temperature. After stirring the reaction mixture at 80° C. for overnight, the reaction progress was monitored by TLC and LCMS. After completion, the reaction mixture was concentrated and resulting residue was portioned between water and ethyl acetate. The organic layers were separated, washed with water and 1% HCl to remove traces of pyridine, dried over Na₂SO₄ and concentrated. The crude residue was purified by silica gel column chromatography to provide the desired compound.

| No | Structure |
|---|---|
| 1 | 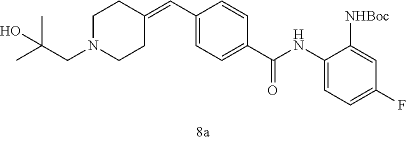 [SMILES: CC(C)(O)CN1CCC(=Cc2ccc(C(=O)Nc3ccc(F)cc3NC(=O)OC(C)(C)C)cc2)CC1] |
| 2 | 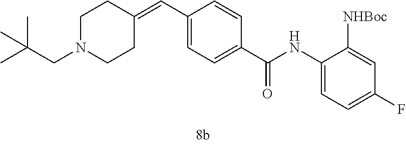 [SMILES: CC(C)(C)CN1CCC(=Cc2ccc(C(=O)Nc3ccc(F)cc3NC(=O)OC(C)(C)C)cc2)CC1] |
| 3 | 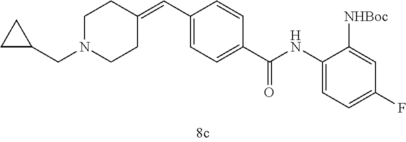 [SMILES: CC(C)(C)OC(=O)Nc1cc(F)ccc1NC(=O)c1ccc(C=C2CCN(CC3CC3)CC2)cc1] |

Step 8: Synthesis of compound 9a-c: To a stirred solution of Boc compound 8 (1 eq) in 1,4-dioxane, 4 M HCl in dioxane was added and the reaction was stirred at room temperature for 1 h. After completion, the reaction mixture was concentrated and the resulting residue was triturated with n-pentane and dried under vacuum to give the desired compound as HCl salt.

| No | Structure |
|---|---|
| 1 | 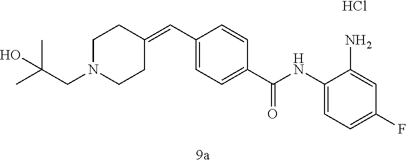 [SMILES: Cl ; CC(C)(O)CN1CCC(=Cc2ccc(C(=O)Nc3ccc(F)cc3N)cc2)CC1] |
| 2 | 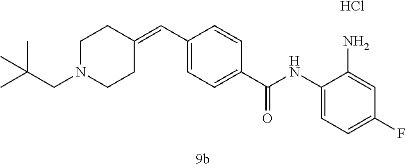 [SMILES: Cl ; CC(C)(C)CN1CCC(=Cc2ccc(C(=O)Nc3ccc(F)cc3N)cc2)CC1] |
| 3 | 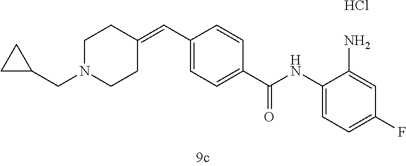 [SMILES: Cl ; Nc1cc(F)ccc1NC(=O)c1ccc(C=C2CCN(CC3CC3)CC2)cc1] |

Step 10: Synthesis of compound B: To a stirred solution of compound A (5 g, 1 eq) in THF (100 mL), DMAP (0.312 g, 0.08 eq) and Boc anhydride (17.4 g, 2.5 eq) dissolved in THF was added. The resulting reaction mixture was stirred at room temperature for 16 h. The progress of the reaction was monitored by TLC. After completion, the reaction was quenched with sat. NaHCO₃ solution and extracted with ethyl acetate. The organic layers were separated, washed with water and dried over Na₂SO₄ and concentrated to provide the desired compound B.

Step 11: Synthesis of tert-butyl (5-fluoro-2-nitrophenyl)carbamate (C): To a stirred solution of compound B (11 g, 1 eq) in DCM (110 mL) at 0° C., TFA (3.5 mL, 1.5 eq) was added. The resulting reaction mixture was stirred at room temperature for 1 h. The progress of the reaction was monitored by TLC. After completion, the reaction was quenched with sat. NaHCO₃ solution and extracted with ethyl acetate. The organic layers were separated, washed with water and dried over Na₂SO₄ and concentrated to provide the desired compound C.

Step 12: Synthesis of tert-butyl (2-amino-5-fluorophenyl)carbamate (D): To a stirred solution of compound C (4 g, 1 eq) in dry THF (100 mL) under argon atmosphere, Raney Ni (2 g) was added and the reaction mixture was stirred under hydrogen atmosphere (balloon pressure) at room temperature for overnight. The progress of the reaction was monitored by TLC. After completion, the reaction mixture was filtered through a pad of celite, the filtrate was evaporated under reduced pressure and the resulting residue was triturated with diethyl ether and n-pentane and then dried under vacuum to afford the title compound D.

Step 9: Synthesis of N-(2-amino-4-fluorophenyl)-4-((1-(2-hydroxy-2-methylpropyl)piperidin-4-yl)methyl)benzamide (Compound 161): To a stirred solution of 9c (0.05 g, 1 eq) in methanol (1 mL), methanolic HCl (1 mL) and 10% Pd/C (5 mg) was added and the reaction mixture was stirred under hydrogen atmosphere (balloon pressure) at room temperature for 4 h. The progress of the reaction was monitored by TLC. After completion, the reaction mixture was filtered through a pad of celite, the filtrate was evaporated under reduced pressure and the resulting residue was triturated with diethyl ether and n-pentane and then dried under vacuum to afford the title compound.

¹H NMR (400 MHz, DMSO-d6) δ 9.84 (s, 1H), 9.18 (bs, 1H), 7.98 (d, J=7.6 Hz, 2H), 7.33 (d, J=7.6 Hz, 2H), 7.22-7.20 (m, 1H), 6.76-6.74 (m, 1H), 6.59-6.57 (m, 1H), 3.57-3.54 (m, 2H), 3.29-3.25 (m, 1H), 3.16-3.14 (m, 2H), 3.05-2.89 (m, 4H), 2.72-2.65 (m, 1H), 2.60 (d, J=5.9 Hz, 1H), 1.78-1.68 (m, 4H), 1.25 (s, 6H); LCMS Calculated for C₂₃H₃₀FN₃O₂ (free base): 399.23; Observed: 399.95 (M+1)⁺.

Step 9: Synthesis of N-(2-amino-4-fluorophenyl)-4-((1-(cyclopropylmethyl) piperidin-4-yl)methyl)benzamide (Compound 163): To a stirred solution of 9b (0.05 g, 1 eq) in methanol (1 mL), 10% Pd/C (5 mg) was added and the reaction mixture was stirred under hydrogen atmosphere (balloon pressure) at room temperature for 4 h. The progress of the reaction was monitored by TLC. After completion, the reaction mixture was filtered through a pad of celite, the filtrate was evaporated under reduced pressure and the resulting residue was triturated with diethyl ether and n-pentane and then dried under vacuum to afford the title compound.

¹H NMR (400 MHz, DMSO-d6) δ 10.19 (s, 1H), 9.80 (s, 1H), 7.97 (d, J=8.0 Hz, 2H), 7.33 (d, J=7.6 Hz, 2H), 7.21 (t, J=7.6 Hz, 1H), 6.74-6.71 (m, 1H), 6.57-6.55 (m, 1H), 3.57-3.44 (m, 2H), 2.92-2.73 (m, 4H), 2.67-2.63 (m, 2H), 1.87-1.70 (m, 3H), 1.64-1.49 (m, 2H), 1.19-1.02 (m, 1H), 0.63-0.61 (m, 2H), 0.45-0.31 (m, 2H); LCMS Calculated for C₂₃H₂₈FN₃O (free base): 381.22; Observed: 381.95 (M+1)⁺.

Step 9: Synthesis of N N-(2-amino-4-fluorophenyl)-4-((1-neopentylpiperidin-4-yl)methyl)benzamide (Compound-162): To a stirred solution of 9 (0.1 g, 1 eq) in methanol (5 mL), 10% Pd/C (10 mg) was added and the reaction mixture was stirred under hydrogen atmosphere (balloon pressure) at room temperature for 4 h. The progress of the reaction was monitored by TLC. After completion, the reaction mixture was filtered through a pad of celite, the filtrate was evaporated under reduced pressure and the resulting residue was purified by combiflash and SFC chromatography to afford the desired compound.

¹H NMR (400 MHz, DMSO-d6) δ 9.51 (s, 1H), 7.89 (d, J=8.0 Hz, 2H), 7.28 (d, J=8.0 Hz, 2H), 7.10 (t, J=8.4 Hz, 1H), 6.55-6.52 (m, 1H), 6.38-6.33 (m, 1H), 5.20 (s, 2H), 2.74-2.70 (m, 2H), 2.58-2.56 (m, 2H), 2.10 (t, J=11.2 Hz, 2H), 1.49-1.46 (m, 3H), 1.24-1.22 (m, 2H), 1.04-1.02 (m, 1H), 0.82 (s, 9H), 1H merged in solvent peak; LCMS Calculated for C₂₄H₃₂FN₃O (free base): 397.25; Observed: 398.37 (M+1)⁺.

### Synthetic Scheme for Compound 146 and Compound 147

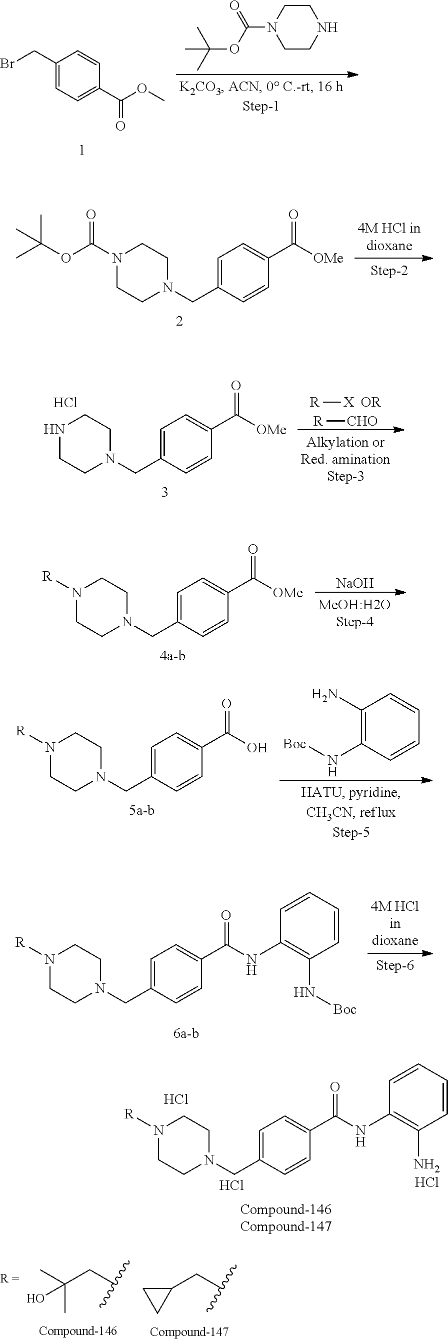

Step 1: Synthesis of tert-butyl 4-(4-(methoxycarbonyl)benzyl)piperazine-1-carboxylate (2): To a stirred solution of tert-butyl piperazine-1-carboxylate (2.92 g, 1.2 eq) and potassium carbonate (3.33 g, 3 eq) in ACN (25 mL), compound 1 (3 g, 1 eq) was added. The reaction mixture was stirred at room temperature for 16 h. The progress of reaction was monitored by TLC. After completion, the reaction mixture was diluted with water and extracted with ethyl acetate. The combined organic extracts were washed with water, brine, dried over anhydrous Na₂SO₄, filtered and concentrated under reduced pressure to provide a crude residue which was purified by silica gel column chromatography to afford compound 2.

Step 2: Synthesis of methyl 4-(piperazin-1-ylmethyl)benzoate hydrochloride (3): To a stirred solution of Boc compound 2 (4 g, 1 eq) in 1,4-dioxane (2 mL), 4 M HCl in dioxane was added and reaction was stirred at room temperature for 1 h. After completion of reaction, the reaction mixture was concentrated and the resulting residue was triturated with n-pentane and dried under vacuum to give the desired compound 3.

Step 3: Synthesis of compound 4a: To a solution of compound 3 (1 eq) in 5 vol of ethanol was added TEA (3 eq) followed by 2,2-dimethyloxirane (2.5 eq) at room temperature and the reaction mixture was heated at 90° C. for 4 h. The progress of reaction was monitored by TLC. After completion, the reaction mixture was allowed to cool, concentrated to give a crude compound which was purified by silica gel column chromatography.

Step 3: Synthesis of compound 4b: To a stirred solution of compound 3 (1 eq) and cesium carbonate (3 eq) in DMF (10 vol), corresponding alkyl halide (1.1 eq) was added. The reaction mixture was heated at 80° C. for 12 h. The progress of reaction was monitored by TLC. After completion, the reaction mixture was poured into ice-water and extracted with ethyl acetate. The combined organic extracts were washed with water, brine, dried over anhydrous Na₂SO₄, filtered and concentrated under reduced pressure to provide a crude residue which was purified by silica gel column chromatography

| No | Structure |
|---|---|
| 1 | 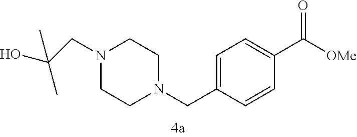 [SMILES: COC(=O)c1ccc(CN2CCN(CC(C)(C)O)CC2)cc1] |
| 2 | 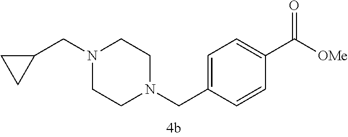 [SMILES: COC(=O)c1ccc(CN2CCN(CC3CC3)CC2)cc1] |

Step 4: Synthesis of compound 5a-b: To stirred solution of ester compound in Methanol: Water (1:1) was added NaOH (1.5 eq) at room temperature. The above mixture was heated to 90 oC for 5h. The progress of the reaction was monitored by TLC. After completion of reaction, the reaction mixture was concentrated and the resulting residue was dissolved in water and washed with diethyl ether. The aqueous layer was neutralized to pH=7 using 1 N HCl at 0° C. The solid obtained was filtered, washed with water and dried under vacuum to provide the desired compound.

| No | Structure |
|---|---|
| 1 | 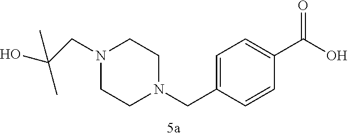 [SMILES: CC(C)(O)CN1CCN(Cc2ccc(C(=O)O)cc2)CC1] |
| 2 | 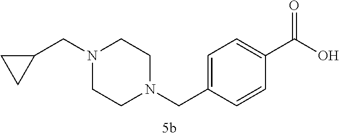 [SMILES: O=C(O)c1ccc(CN2CCN(CC3CC3)CC2)cc1] |

Step 5: Synthesis of compound 6a-b: To a stirred solution of acid compound (1 eq) and amine (1.1 eq) in ACN (10 vol.), pyridine (5eq) and HATU (1.5 eq) was added at room temperature. After stirring the reaction mixture at 80° C. for overnight, the reaction progress was monitored by TLC and LCMS. After completion, the reaction mixture was concentrated and resulting residue was partitioned between water and ethyl acetate. The organic layers were separated, washed with water and 1% HCl to remove traces of pyridine, dried over Na₂SO₄ and concentrated. The crude residue was purified by silica gel column chromatography to provide the desired compound.

| No | Structure |
|---|---|
| 1 | 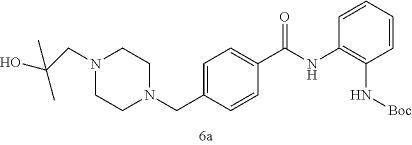 [SMILES: CC(C)(O)CN1CCN(Cc2ccc(C(=O)Nc3ccccc3NC(=O)OC(C)(C)C)cc2)CC1] |
| 2 | 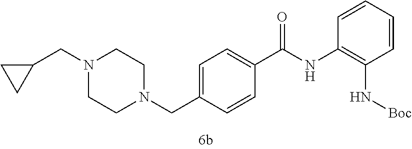 [SMILES: CC(C)(C)OC(=O)Nc1ccccc1NC(=O)c1ccc(CN2CCN(CC3CC3)CC2)cc1] |

Step 6: Synthesis of N-(2-aminophenyl)-4-((4-(2-hydroxy-2-methylpropyl)piperazin-1-yl)methyl)benzamide (Compound 146): To a stirred solution of Boc compound 6a (1 eq) in 1,4-dioxane (5 vol.) 4 M HCl in dioxane (5 vol)at room temperature. After completion of reaction, the reaction mixture was concentrated and the resulting residue was triturated with n-pentane and dried under vacuum to provide the desired compound.

¹H NMR (400 MHz, DMSO-d6): δ 10.65 (s, 1H), 8.22 (d, J=7.8 Hz, 2H), 7.82 (d, J=7.8 Hz, 2H), 7.63-7.56 (m, 1H), 7.48 (d, J=7.5 Hz, 1H), 7.41-7.28 (m, 2H), 4.49 (s, 2H), 3.76-3.66 (m, 4H), 3.62-3.56 (m, 4H), 3.21-3.16 (m, 2H), 1.26 (s, 6H); LCMS Calculated for C₂₂H₃₀N₄O₂ for free base: 382.24; Observed: 382.90 (M+1)⁺.

Step 6: Synthesis of N-(2-aminophenyl)-4-((4-(cyclopropylmethyl)piperazin-1-yl)methyl)benzamide (Compound 147): To a stirred solution of Boc compound 6b (1 eq) in 1,4-dioxane (5 vol.) 4 M HCl in dioxane (5 vol.)at room temperature. After completion of reaction, the reaction mixture was concentrated and the resulting residue was triturated with n-pentane and dried under vacuum to provide the desired compound.

¹H NMR (400 MHz, DMSO-d6): δ 11.72 (s, 1H), 10.53 (s, 1H), 8.18 (d, J=7.8 Hz, 2H), 7.80 (d, J=7.8 Hz, 2H), 7.56-7.49 (m, 1H), 7.42-7.34 (m, 1H), 7.31-7.30 (m, 2H), 4.42 (s, 2H), 3.72-3.70 (m, 2H), 3.55-3.44 (m, 6H), 3.10-3.02 (m, 2H), 1.11-1.09 (m, 1H), 0.63-0.62 (m, 2H), 0.41-0.39 (m, 2H); LCMS Calculated for C₂₂H₂₈N₄O for free base: 364.23; Observed: 365.15 (M+1)⁺.

### Synthetic Scheme for Compound-555

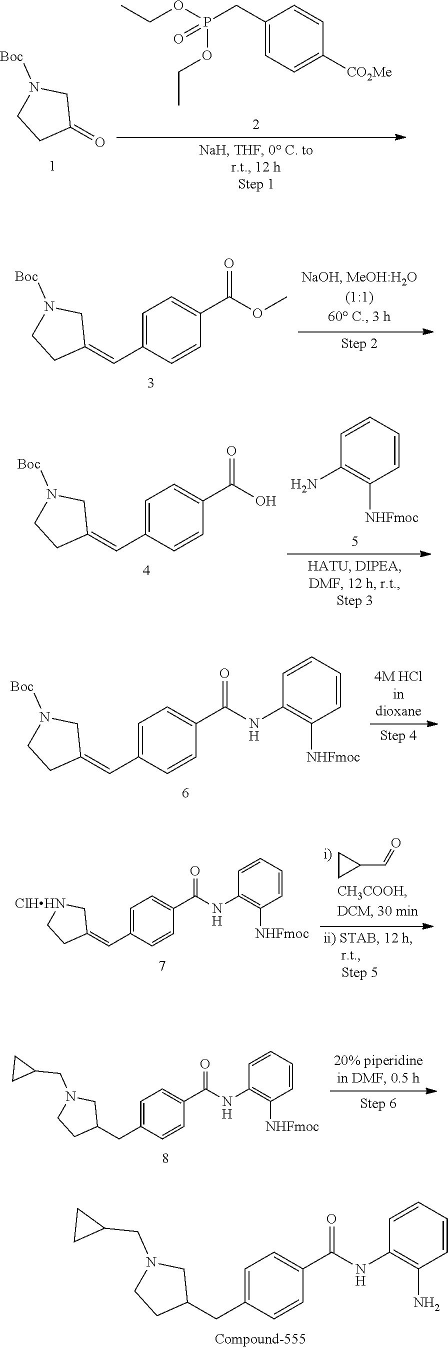

Step 1: Synthesis of tert-butyl (Z)-3-(4-(methoxycarbonyl)benzylidene)pyrrolidine-1-carboxylate (3):

Step 1a: Synthesis of methyl 4-((diethoxyphosphoryl)methyl)benzoate (2): A mixture of methyl 4-(bromomethyl)benzoate (10 g, 43.66 mmol, 1 eq) and triethyl phosphite (10.8 g, 65.50 mmol, 1.5 eq) was heated in seal tube at 130° C. for 12 h. The progress of the reaction was monitored by TLC. After completion, the reaction mixture was concentrated under reduced pressure. The crude residue was purified by silica gel column chromatography to afford the title compound 2.

Step 1b: Synthesis of tert-butyl (Z)-3-(4-(methoxycarbonyl)benzylidene) pyrrolidine-1-carboxylate (3): To a stirred solution of compound 2 (17 g, 59.39 mmol, 1.1 eq) in anhydrous THF (100 mL) at 0° C., NaH (3.88 g, 60% w/w in mineral oil, 80.98 mmol, 1.5 eq) was added under N₂ atmosphere. After stirring the reaction mixture for 30 min, a solution of compound 1 (10 g, 53.99 mmol, 1 eq) in THF was added at 0° C. The reaction mixture was then stirred at room temperature for 12 h. The progress of reaction was monitored by TLC. After completion, the reaction mixture was diluted with water and extracted with ethyl acetate. The combined organic extracts were washed with water, brine, dried over anhydrous Na₂SO₄, filtered, and concentrated under reduced pressure to afford a crude residue which was purified by silica gel column chromatography to afford the title compound 3.

Step 2: Synthesis of (Z)-4-((1-(tert-butoxycarbonyl)pyrrolidin-3-ylidene)methyl) benzoic acid (4): To a stirred solution of compound 3 (4 g, 12.62 mmol, 1 eq) in methanol: water (1:1, 20 mL), NaOH (0.757 g, 18.92 mmol, 1.5 eq) was added and the reaction mixture was stirred at 60° C. for 3 h. The progress of reaction was monitored by TLC. After completion, methanol was removed under reduced pressure and reaction mixture was acidified with 2 N HCl up to pH˜5, during which a solid precipitated. The obtained solid was filtered, washed with water, and dried under reduced pressure to afford the title compound 4.

Step 3: Synthesis of tert-butyl (Z)-3-(4-((2-((((9H-fluoren-9-yl)methoxy)carbonyl) amino)phenyl)carbamoyl)benzylidene)pyrrolidine-1-carboxylate (6):

Step 3a: Synthesis of (9H-fluoren-9-yl)methyl (2-aminophenyl)carbamate (5): To a stirred solution of benzene-1,2-diamine (5 g, 46.29 mmol, 1 eq) in DMF (20 mL), a solution of FmocOSu (15.60 g, 46.29 mmol, 1 eq) in DMF (50 mL) was added slowly. The reaction mixture was stirred at room temperature for 2 h. The progress of the reaction was monitored by TLC. After completion, the reaction mixture was quenched with water. The precipitated solid was collected by filtration and dried under reduced pressure. The crude compound was purified by silica gel column chromatography to afford the title compound 5.

Step 3b: tert-butyl (Z)-3-(4-((2-((((9H-fluoren-9-yl)methoxy)carbonyl)amino) phenyl)carbamoyl)benzylidene)pyrrolidine-1-carboxylate (6): To a stirred solution of compound 4 (3.8 g, 12.5 mmol, 1 eq) and compound 5 (4.96 g, 15.04 mmol, 1.2 eq) in DMF (20 mL), DIPEA (5.39 mL, 31.35 mmol, 2.5 eq) was added and stirred for 10 min. To this solution, HATU (7.15 g, 18.31 mmol, 1.5 eq) was added slowly and the reaction mixture was stirred at room temperature for 12 h. The progress of the reaction was monitored by TLC. After completion, the reaction mixture was diluted with water and extracted with ethyl acetate. The combined organic extracts were washed with water, brine, dried over anhydrous Na₂SO₄, filtered, and concentrated under reduced pressure. The crude product was purified by column chromatography to afford the title compound 6.

Step 4: Synthesis of (9H-fluoren-9-yl)methyl (Z)-(2-(4-(pyrrolidin-3-ylidenemethyl)benzamido)phenyl)carbamate hydrochloride (7): To a stirred solution of compound 6 (2.1 g, 3.41 mmol, 1 eq) in 1,4 dioxane (5 mL) at 0° C., 4 M HCl in dioxane (15 mL) was added and the reaction mixture was stirred at room temperature for 3 h. The progress of the reaction was monitored by TLC. After completion, the reaction mixture was concentrated under reduced pressure. The residue was diluted with saturated NaHCO₃ solution and extracted with ethyl acetate. The combined organic extracts were washed with water, brine, dried over anhydrous Na₂SO₄, filtered, and concentrated under reduced pressure to afford the compound 7 as HCl salt.

Step-5: Synthesis of (9H-fluoren-9-yl)methyl (2-(4-((1-(cyclopropylmethyl) pyrrolidin-3-yl)methyl)benzamido)phenyl)carbamate (8): To a stirred solution of amine compound 7 (0.2 g, 0.362 mmol, 1 eq) and cyclopropanecarbaldehyde (0.03 g, 0.435 mmol, 1.2 eq) in DCM (10 mL), acetic acid (0.065 g, 1.086 mmol, 3 eq) was added and stirred at room temperautre for 30 min. To this solution, sodium triacetoxyborohydride (STAB) (0.115 g, 0.543 mmol, 1.5 eq) was added and stirring was continued at room temperature for 12 h. The reaction progress was monitored by TLC and LCMS. After completion, the reaction mixture was diluted with saturated NaHCO₃ solution and extracted with DCM. The combined organic extracts were washed with water, brine, dried over anhydrous Na₂SO₄, filtered, and concentrated under reduced pressure. The crude product was purified by column chromatography to afford the title compound 8.

Step-6: Synthesis of N-(2-aminophenyl)-4-((1-(cyclopropylmethyl)pyrrolidin-3-yl)methyl)benzamide (Compound-555): A solution of compound 8 (0.1 g, 0.175 mmol, 1 eq) in 20% piperidine in DMF (3 mL) was a stirred at room temperature for 30 min. The progress of the reaction was monitored by TLC. After completion, the reaction mixture was quenched with ice cold water. The precipitated solid was collected by filtration, then the solid was washed with water, pentane, and concentrated under reduced pressure. The residue was purified by silica gel column chromatography followed by preparatory TLC to afford compound Compound-555.

¹H NMR (400 MHz, DMSO-d6) δ 9.60 (s, 1H), 7.91 (d, J=7.6 Hz, 2H), 7.34 (d, J=8.0 Hz, 2H), 7.12 (d, J=7.6 Hz, 1H), 6.94 (t, J=7.2 Hz, 1H), 6.75 (d, J=7.6 Hz, 1H), 6.56 (t, J=7.2 Hz, 1H), 4.86 (s, 2H), 3.53-3.47 (m, 1H), 3.17-3.15 (m, 2H), 2.97-2.95 (m, 2H), 2.80-2.74 (m, 2H), 2.00-1.84 (m, 1H), 1.67-1.50 (m, 1H), 0.88-0.77 (m, 1H), 0.56-0.54 (m, 2H), 0.33-0.32 (m, 2H), 2H merged in solvent peak; LCMS 350.05 (M+1)⁺.

### Synthetic Scheme for Compound 556

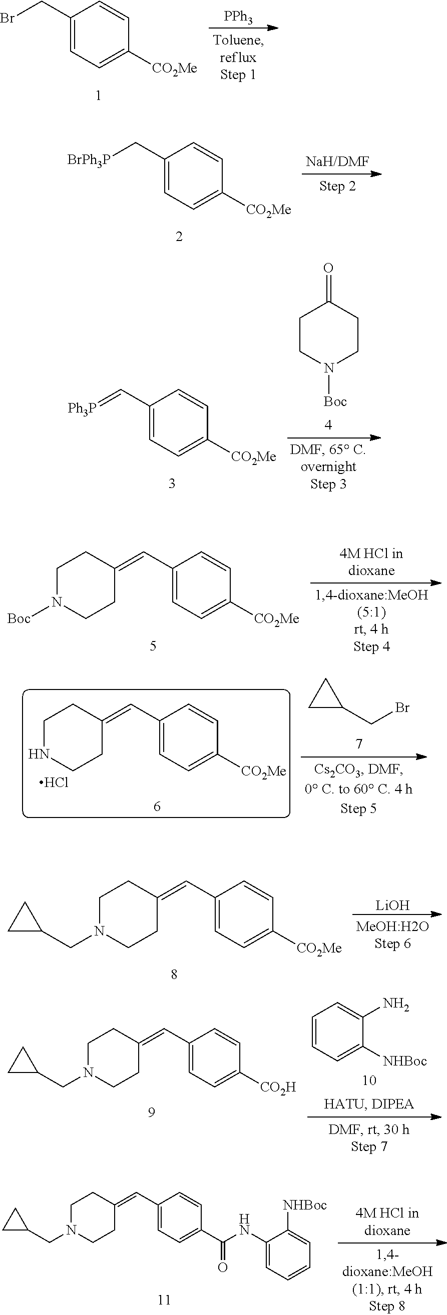

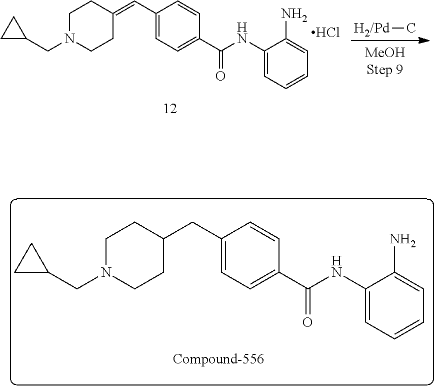

Step 1: Synthesis of methyl 4-((bromotriphenyl-⊐⁵-phosphanyl) methyl)benzoate (2): To a stirred solution of compound 1 (75 g, 326 mmol, 1 eq) in toluene (1 L) triphenyl phosphine (85.5 g, 326 mmol, 1 eq) was added and the reaction mixture was heated at reflux for 7 h. After 7 h, the reaction mixture was allowed to cool to room temperature. The precipitate formed was filtered, washed with toluene followed by hexane, and dried under vacuum to afford compound 2.

Step 2 & 3: Synthesis of tert-butyl 4-(4-(methoxycarbonyl)benzylidene)piperidine-1-carboxylate (5): To a stirred solution of compound 2 (50 g, 102 mmol, 1 eq) in anhydrous DMF (500 mL) at 0° C., NaH (4.9 g, 60% w/w in mineral oil, 122 mmol, 1.2 eq) was added under N₂ atmosphere. After stirring the reaction mixture for 30 min, a solution of compound 4 (24.3 g, 122 mmol, 1.2 eq) in DMF was added and the reaction mixture was then heated at 65° C. for 12 h. The progress of reaction was monitored by TLC. After completion, the reaction mixture was cooled to 0° C., diluted with ice-cold water under stirring and the precipitate formed was filtered. The solid obtained was taken in ethyl acetate, stirred for 10 min and the resulting mixture was filtered. The filtrate was washed with brine and water. The organic layer was separated, dried over anhydrous Na₂SO₄, and evaporated under reduced pressure to obtain the product, which was purified by silica gel column chromatography to afford compound 5.

Step 4: Synthesis of methyl 4-(piperidin-4-ylidenemethyl)benzoate hydrochloride (6): To a stirred solution of compound 5 (30 g, 90.6 mmol, 1 eq) in 1,4 dioxane: MeOH (210 mL: 40 mL) at 0° C., 4 M HCl in dioxane (85 mL) was added and the reaction mixture was stirred at room temperature for 4 h. The progress of the reaction was monitored by TLC. After completion, the reaction mixture was diluted with diethyl ether. The solid obtained was filtered and washed with diethyl ether. The residue was dried under reduced pressure to afford the title compound 6 as a hydrochloride salt.

Step 5: Synthesis of methyl 4-((1-(cyclopropylmethyl)piperidin-4-ylidene)methyl) benzoate (8): To a stirred solution of compound 6 (12 g, 45 mmol) in DMF (250 mL) at 0° C., cyclopropyl methylene bromide 7 (5 mL, 50 mmol) and Cs₂CO₃ (29.3 g, 90 mmol) were added. The reaction mixture was stirred at 60° C. for 4 h. The reaction progress was monitored by TLC and LCMS. After completion, the reaction mixture was diluted with water and extracted with ethyl acetate. The combined organic extracts were washed with water, brine, dried over anhydrous Na₂SO₄, filtered, and concentrated under reduced pressure. The residue was purified by silica gel column chromatography to afford compound 8.

Step 6: Synthesis of 4-((1-(cyclopropylmethyl)piperidin-4-ylidene)methyl)benzoic acid (9): To a stirred solution of compound 8 (18.5 g, 64.91 mmol, 1 eq) in methanol (300 mL), aqueous LiOH (4.1 g, 97.36 mmol in 60 mL water) was added and the reaction mixture was stirred at room temperature for 12 h. The progress of reaction was monitored by TLC. After completion, methanol was removed under reduced pressure and reaction mixture was acidified with 2 N HCl until pH=3-4. The resulting solid obtained was filtered, washed with 2 N HCl (2 L) & dried. The solid was further washed with diethyl ether (1 L), azeotroped with toluene (3×500 mL), and dried under vacuum to afford compound 9.

Step 7: Synthesis of tert-butyl (2-(4-((1-(cyclopropylmethyl)piperidin-4-ylidene) methyl)benzamido)phenyl)carbamate (11):

Step 7a: Synthesis of tert-butyl (2-aminophenyl)Carbamate (10): To a stirred solution of benzene-1,2-diamine (54 g, 500 mmol, 1 eq) in THF (500 mL), (Boc)₂O (109.09 g, 500 mmol) in 150 mL THF was added slowly at 0° C. The reaction mixture was slowly warmed to room temperature and stirred for 12 h. The progress of the reaction was monitored by TLC. After completion, the reaction mixture was concentrated and resulting residue was purified by silica gel column chromatography to afford compound 10.

Step 7b: Synthesis of tert-butyl (2-(4-((1-(cyclopropylmethyl)piperidin-4-ylidene)methyl)benzamido)phenyl)carbamate (11): To a stirred solution compound 9 (8.8 g, 32.47 mmol, 1 eq) and compound 10 (8.1 g, 38.96 mmol, 1.2 eq) in DMF (100 mL), DIPEA (23 mL, 129.8 mmol, 4 eq) was added and stirred for 10 min. To this solution, HATU (18.5 g, 48.70 mmol, 1.5 eq) was added slowly and the reaction mixture was stirred at room temperature for 12 h. The progress of the reaction was monitored by TLC. After completion, the reaction mixture was diluted with water and extracted with ethyl acetate. The combined organic extracts were washed with water, brine, dried over anhydrous Na₂SO₄, filtered and concentrated in vacuo. The residue was purified by column chromatography to afford compound 11.

Step 8: Synthesis of N-(2-aminophenyl)-4-((1-(cyclopropylmethyl)piperidin-4-ylidene)methyl)benzamide (12): To a stirred solution of compound 11 (7 g, 15.18 mmol, 1 eq) in 1,4 dioxane:MeOH (21 mL: 7 mL) at 0° C., 4 M HCl in dioxane (21 mL) was added and the reaction mixture was stirred at room temperature for 4 h. The progress of the reaction was monitored by TLC. After completion, the reaction mixture was diluted with 1,4 dioxane, stirred for 15 min and filtered. The residue was taken in diethyl ether, stirred for 15 min and again filtered. The solid compound was dried under reduced pressure to afford compound 12 as HCl salt.

Step 9: Synthesis of N-(2-aminophenyl)-4-((1-(cyclopropylmethyl)piperidin-4-yl)methyl)benzamide (Compound-556): To a stirred solution of 12 (0.1 g, 0.216 mmol, 1 eq) in methanol (10 mL), 10% Pd/C (50 mg) was added and the reaction mixture was stirred under hydrogen atmosphere (balloon pressure) at room temperature for 2 h. The progress of the reaction was monitored by TLC. After completion, the reaction mixture was filtered through a pad of celite and the filtrate was concentrated under reduced pressure. The crude product was purified by silica gel column chromatography to afford Compound-556.

¹H NMR (400 MHz, DMSO-d6) δ 9.58 (s, 1H), 7.90 (d, J=8.4 Hz, 2H), 7.29 (d, J=8.4 Hz, 2H), 7.15 (d, J=8.0 Hz, 1H), 6.96 (t, J=8.0 Hz, 1H), 6.77 (d, J=8.4 Hz, 1H), 6.59 (t, J=7.6 Hz, 1H), 4.85 (brs, 2H), 3.01-2.93 (m, 2H), 2.59-2.57 (m, 2H), 2.25-2.23 (m, 2H), 2.01-1.95 (m, 2H), 1.58-1.54 (m, 3H), 1.26-1.23 (m, 2H), 0.82-0.81 (m, 1H), 0.48-0.45 (m, 2H), 0.09-0.05 (m, 2H); LCMS: 364.15 (M+1)⁺.

### HDAC Enzyme Inhibition

The HDAC activity inhibition assay is performed as follows to determine the ability of a test compound to inhibit HDAC enzymatic activity. Serial dilutions of HDAC inhibitors are prepared in HDAC assay buffer (25 mM Tris/HCl, pH 8.0, 137 mM NaCl, 2.7 mM KCl, 1 mM MgCl₂, pH 8) in 96-well assay plates (Fisher scientific, #07-200-309) and pre-incubated for 2 hours at room temperature in the presence of 125 μg/ml BSA and purified HDAC1 (BPS Bioscience, San Diego, Calif., #50051), HDAC2 (BPS Bioscience, #50053), or HDAC3/NcoR2 (BPS Bioscience, #50003) at concentrations of 1.25, 1.32, and 0.167 μg/mL, respectively. Following pre-incubation, Fluor-de-Lys™ substrate (Enzo Life Sciences, Plymouth Meeting, Pa., BML-KI104-0050) is added to a final concentration of 10 μM and plates are further incubated for 30 minutes at room temperature. The enzymatic reaction is stopped by addition of Trichostatin A (Sigma-Aldrich, St Louis, Mo., # T8552, final concentration: 100 nM) and trypsin (MP Biomedicals, Solon, Ohio, #02101179) are added to reach a final concentration of 100 μg/mL. After a 15 minute incubation at room temperature, fluorescence is recorded using a Spectramax M2 fluorometer (Molecular Devices, Sunnyvale, Calif.) with excitation at 365 nm and emission at 460 nm. IC₅₀ values are calculated by using a sigmoidal dose-response (variable slope) equation in GraphPad Prism® 5 for Windows (Graph Pad Software, La Jolla, Calif.).

### Acid Stability Determination

A 1000 solution of test compound is prepared by dilution of a 10 mM DMSO stock solution in a 0.01 M solution of HCl in deionized water. Immediately after mixing, an aliquot (100 μL) is sampled and analyzed by HPLC/UV. The area under the compound peak is determined and used as the time zero reference point. The remainder of the acid sample is incubated at 50° C. and samples were taken after 2, 4, and 24 or 30 hours of incubation. These are analyzed by the same HPLC/UV method and the area of the peak corresponding to the test compound is measured. Percent remaining at a given time point is then calculated as the ratio of the area under the peak after incubation to that at time zero times 100. In those embodiments where a 30 hour time point is recorded, the percent remaining at 24 hours is obtained by interpolation of the percent remaining versus time curve assuming a unimolecular process, i.e. a monoexponential decay.

### Brain Penetration Studies

Test compounds are prepared at either 0.5 mg/ml or 5 mg/ml in 30% hydroxypropyl-β-cyclodextrin, 100 mM sodium acetate pH 5.5, 5% DMSO. Rats or C57/BL6/J mice are dosed s.c. at 5 mg/kg or 50 mg/kg, or i.v. at 5 mg/kg. Animals are euthanized at pre-dose, 5, 15, 30 min, 1, 2 and 4 hours post-dose and plasma and brain obtained. Three animals per dose per time points are used. The levels of compound in the plasma and brain are determined by standard LC/MS/MS methods. Brain/plasma ratio (BPR) is calculated as the ratio of the Cₘₐₓ(brain)/Cₘₐₓ(plasma).

### In-cell Deacetylase Inhibition Assay (DAC Assay)

GM 15850(lymphoblastoid cells line) cells are seeded in 96-well plates at an appropriate density (100,000 cells/well) in 90 μL RPM11640 medium containing 10% v/v fetal bovine serum (FBS), 1% v/v penicillin/streptomycin, and 1% v/v L-glutamine. Compound dilutions are made in 100% DMSO followed by parallel dilution in media with 2% DMSO. 10 μl of the compound dilutions are added to the cells to achieve the desired concentrations. The final concentration of DMSO in each well is 0.2%. The cells are incubated for 4 h at 37° C. with 5% CO₂. After incubation, the cells are centrifuged down and the supernatant removed. The cell pellets are washed with 100 μL phosphate-buffered saline (PBS) and then lysed with 45 μL lysis buffer (HDAC assay buffer at pH 8.0 (25 mM Tris/HCl, 137 mM NaCl, 2.7 mM KCl, 1 mM MgCl₂) +1% v/v Igepal CA-630). To initiate the reaction, the HDAC substrate KI-104 (Enzo Life Sciences, Farmingdale, N.Y.) is added to a final concentration of 50 μM. The reaction is stopped after 30 min incubation by addition of 50 μL developer (6 mg/mL trypsin in HDAC assay buffer). The reaction is allowed to develop for 30 min at room temperature and the fluorescence signal is detected using a fluorometer (Spectramax M2, Molecular Devices, Sunnyvale, Calif.) with excitation and emission wavelengths of 360 nm and 470 nm respectively. The data are fitted to a sigmoidal dose response equation with variable slope in GraphPad Prism 5.0 (GraphPad Software, La Jolla, Calif.) to determine IC₅₀. Bottom and top of the curve are fixed to the average fluorescence response of control wells with no cells and cells but no compound respectively.

### Cell Proliferation Assay

HCT116 cells (5000 cells/well) in 80 μL McCoy's 5A medium containing 10% v/v FBS, 1% v/v penicillin/streptomycin and 1% v/v L-glutamine are incubated in 96-well plates with compounds at various concentrations for 72 h at 37° C. in a 5% CO₂ atmosphere. The compound dilutions are made in 100% DMSO followed by parallel dilutions in media. The final concentration of DMSO in each well is 0.05%. After 72 h, 20 μL of Cell titer 96 aqueous one solution (Promega Corporation, Madison, Wis.) are added to the cells and the plate is incubated at 37° C. for another 4 h. The absorbance at 490 nm is then recorded on a 96-well plate reader (Spectramax M2, Molecular Devices, Sunnyvale, Calif.). Data analysis is performed in Microsoft Excel (Microsoft Corp, Redmond, Wash.).((O.D. sample—average O.D. positive control)/(average O.D. negative control—average O.D. positive control))*100, where O.D. is the measured absorbance, O.D. positive control is the absorbance from cells incubated with trichostatin A at 5 μM and O.D. negative control is the absorbance measured from cells incubated without any compound, is plotted against compound concentration and an IC₅₀ is determined by graphical interpolation of the concentration required for 50% inhibition of cell growth.

Effect of HDAC Inhibitors on Frataxin (FXN) mRNA Expression

Method: mRNA quantification of compound-treated iPSC derived neuronal cells Neuronal stem cells were cultured in Neurobasal A medium (Life technologies #10888022) supplemented with N2, B27 (Life technologies #17502-048 and #17504-044), L-glutamine (Life technologies #25030081), supplemented with 20 ng/ml EGF (R&D Systems #236-EG) and 20 ng/ml bFGF (BioPioneer # HRP-0011). Neuronal differentiation was initiated by removing growth factors and culturing cells in Neurobasal A with N2 and B27. Cells were allowed to differentiate for 16 days. HDAC inhibitory compound was then added and incubate for 24 h. RNA isolation was performed using the RNeasy Plus mini kit (QIAgen #74134) using a QIAcube instrument per manufacturer's instructions. qRT-PCR was performed using qScript One-Step SYBR Green qRT-PCR Kit (Quanta Biosciences 170-8893BR) with the following conditions: 20 minutes at 50° C., 5 minutes at 95° C., and then 40 cycles of 20 seconds at 95° C., 20 seconds at 55° C., 30 seconds at 72° C. The primer sequences to detect expression of FXN were: 5′-CAGAGGAAACGCTGGACTCT-3′ and 5′-AGCCAGATTTGCTTGTTTGG-3′.

Data for compounds for iPSC fold inductions and cLogP are shown in Table 3. Data for additional compounds for iPSC fold inductions and cLogP are shown in Table 4 The ranges reported for cLogP refer to the following. A<1, 1<B<2, 2<C<3, 3<D<5, E>5. NA refers to “not available.”

TABLE 3

| | Fold | |
|---|---|---|
| Compound | Induction | cLogP |
| 146 (Tri-HCl) | 1.8 | B |
| 147 (Tri-HCl) | 1.8 | C |
| 171 (Di-HCl) | 0.9 | C |
| 172 (Di-HCl) | 1.4 | D |
| 174 (Di-HCl) | 1.1 | D |
| 354 (Free base) | 1.9 | E |
| 175 (Free base) | 1.5 | A |
| 241 (Tri-TFA) | 1.9 | B |
| 238 | 1.5 | C |
| 176 (Di-TFA) | 1.5 | B |
| 161 | 1.2 | D |
| 163 | 1.2 | D |
| 169 | 1.3 | C |
| 162 | 1.3 | D |
| 356 | 2.0 | D |
| 357 | 1.7 | D |
| 359 (Free base) | 1.9 | D |
| 379 (Di-HCl) | 2.1 | D |
| 181 (Free base) | 1.5 | C |
| 472 (Free base) | 1.5 | D |
| 485 (Tri-HCl) | 2.2 | C |
| 486 | 1.9 | B |
| 488(Free base) | 1.5 | C |
| 477 (racemic) | 1.8 | D |
| 478 (racemic) | 2.1 | C |
| 480 | 2.0 | C |
| 479 (Free base) | 2.2 | D |
| 482 (Free base) | 1.7 | C |
| 481 (Free base) | 1.6 | D |
| 489 (Free base) | 1.5 | D |
| 490 (Free base) | 1.4 | C |
| 491 (Free base) | 1.7 | D |
| 478-isomer I | 1.6 | C |
| (Free base) | | |
| 478-isomer II | 1.5 | C |
| (Free base) | | |
| 484 (Free base) | 1.6 | C |
| 492(Free base) | 1.2 | C |
| 483(Free base) | 1.7 | D |
| 477-isomer II | 1.5 | D |
| 477-isomer I | 1.4 | D |
| 487(Free base) | 1.3 | D |

TABLE 4

| | Fold | |
|---|---|---|
| Compound | Induction | cLogP |
| 555 (Free base) | 1.11 | C |
| 556 (Free base) | NA | D |

### Protocol for Compound Stability in Hepatocytes

To assess the stability and metabolism of RGFP compounds in hepatocytes. This assay was designed to evaluate the metabolism of RGFP compounds, following their incubation with human, monkey, dog and rat hepatocytes by monitoring either parent drug disappearance or metabolite appearance using HPLC.

Equipment: Applied Biosystem Triple Quadrupole LC/MS/MS; Ice bucker, timer; 96 well plates; Falcon, Cat #353072; 96 well plates shaker; Various pipettes: 10 μL, 20 μL, 200 μL, and 1000 μL; Test tubes: Catalog # VWR 47729-572, 13×100 mm

Procedure: Turn on the water-bath heater to 37° C. Take out the KHB buffer and make sure it is at room temp before use. Prepare 2.5 mM concentration of RGFP compound in DMSO stock. Add 10 μL of above DMSO stock to 2490 μL KHB buffer; final concentration of RGFP compound will be 10 μM. Pre-warm 45 ml InVitro HT Medium to 37° C. in a sterile 50 mL conical tube. Add 1.0 mL Torpedo Antibiotic Mix per 45 mL InVitro HT medium. Transfer 13 mL of warm HT medium with Antibiotic Mix into a 15 mL conical tube. Carefully remove the hepatocyte vials from liquid nitrogen (liquid phase). Immediately immerse the vial into a 37° C. water bath. Shake gently until the ice melts entirely. Do not keep the cells in 37° C. water bath longer than necessary. Immediately empty contents of the vial into 13 mL of pre-warmed InVitro HT Medium with antibiotics. Rinse the vial with the HT media that you have just transferred the hepatocytes to, in order to ensure complete transfer. Centrifuge the cell suspension at 600 RPM for 5 minutes at room temperature. Discard the supernatant by either pouring in one motion (do not pour partially and re-invert centrifuge tube) or aspirating using a vacuum pump. Add 1.0 mL of KHB (at room temperature) buffer to the tube of hepatocyte pellet. Loosen the cell pellet by gently swirling the centrifuge tube. Transfer 100 μL of above solution to a different tube and add 900 μL of KHB buffer to count the cells. Determine the total cell count and the number of viable cells using the Trypan Blue exclusion method. Once the cell count is obtained, multiply the number by 10 (attributing to the dilution factor). Now add required volume of KHB buffer to the tube containing hepatocytes such that the final count will be 2 million cells/mL. Dispense 50 μL of 2 million cells/ml to a 96 well plate and then add 50 μL of DMSO stock to respective wells (such that, the concentration of RGFP compounds is 5 μM and number of cells are 100000 in each well). Place the plates on a shaker in a 37° C. incubator with 5% CO₂. Separate plates for each time point are advisable (Time points: 0 h, 1 h, 2h, and 6 h). After each time point, add 100 μL of quenching solution.

Quenching solution is an acetonitrile solution containing RGFP531 (10 μM) internal standard, 0.1% formic acid and phenylglyoxol (400 μM). The formic acid and phenylglyoxal is used for the identification and quantification of OPD as mentioned above. Pipette up and down a few times to ensure a complete stop of reaction. Transfer all the solution into a 1.5 mL tube, vortex thoroughly, and centrifuge at 14000 RPM at 4° C. for 5 minutes to precipitate cell debris. Transfer the 150 μL of supernatant to vials for analysis using HPLC.

### Effect of Compounds on Long Term Memory for Object Recognition

Rats or C57BL/6J male mice are handled 1-2 min for 5 days and habituated to the experimental apparatus 5 min a day for 4 consecutive days in the absence of objects. During the training trial, rats or mice are placed in the experimental apparatus with two identical objects and allowed to explore these objects for 3 min, which does not result in short- or long-term memory (Stefanko, et al., 2009). Immediately following training, rats or mice receive subcutaneous injections of either vehicle (20% glycerol, 20% PEG 400, 20% propylene glycol, and 100 mM sodium acetate, pH 5.4), reference compound 1, RGFP109, class I HDAC inhibitor, (3, 10, 30 mg/kg), reference compound 2, RGFP136 (3, 10, 30 mg/kg), or a test compound disclosed herein (3, 10, 30 mg/kg). 24-h later rats or mice are tested for memory retention (5 min) using the object recognition memory task (ORM), in which a familiar object is replaced with a novel one. All training and testing trials are videotaped and analyzed by individuals blind to the treatment condition and the genotype of subjects. A rat or mouse is scored as exploring an object when its head was oriented toward the object within a distance of 1 cm or when the nose is touching the object. The relative exploration time is recorded and expressed by a discrimination index [DI=(tₙₒᵥₑₗ−t_{familiar})/(tₙₒᵥₑₗ+t_{familiar)×}100].

A number of embodiments have been described. Nevertheless, it will be understood that various modifications may be made without departing from the spirit and scope of the disclosure. Accordingly, other embodiments are within the scope of the following claims.

What is claimed is:

1. A compound having a structure of formula (I), or a pharmaceutically acceptable salt thereof: 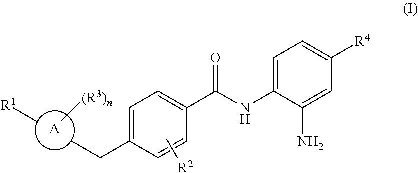
wherein
ring A is a 4-7 membered monocyclic heterocycloalkyl ring or a 7-12 membered spiro heterocycloalkyl ring, wherein ring A contains one nitrogen ring atom and optionally contains one additional ring atom independently selected from O, N, and S;
R¹ is H, C₁₋₆alkyl, C₂₋₆alkenyl, C₁₋₆hydroxyalkyl, C(O)C₁₋₆alkyl, C₀₋₃alkylene-C₃₋₁₀cycloalkyl, or C₀₋₃alkylene-C₂₋₅heterocycloalkyl having 1 or 2 heteroatoms selected from O, S, N, and N(C₁₋₄alkyl);
R² is H, F, Cl, or CH₃;
R³ is C₁₋₃alkyl;
R⁴ is H, F, or Cl; and
n is 0, 1, or 2,
with the proviso that
(a) ring A is not morpholino or thiomorpholino; and
(b) when ring A is piperazinyl, R¹ is C₂₋₆alkenyl, C₁₋₆hydroxyalkyl, C(O)C₁₋₆alkyl, C₀₋₃alkylene-C₃₋₁₀cycloalkyl, or C₀₋₃alkylene-C₂₋₅cycloheteroalkyl having 1 or 2 heteroatoms selected from O, S, N, and N(C₁₋₄alkyl).

2. The compound of claim 1, wherein R¹ is H, C₁₋₆alkyl, C₃₋₆hydroxyalkyl, C₃₋₆alkenyl, or C₁₋₂alkylene-C₃₋₁₀cycloalkyl; R² is H; R³, if present, is CH₃; and R⁴ is H.

3. The compound of claim 1 or 2, wherein ring A is a 7-12 membered spiro heterocycloalkyl ring containing one or two nitrogen ring atoms or one nitrogen ring atom and one oxygen ring atom.

4. The compound of claim 1 or 2, wherein ring A is a 4-7 membered monocyclic heterocycloalkyl ring containing one or two nitrogen ring atoms.

5. The compound of claim 1 or 2, wherein ring A comprises piperidinyl, piperazinyl, azetidinyl, azepanyl, pyrrolidinyl, or diazepanyl.

6. The compound of claim 5, wherein ring A comprises piperidinyl or piperazinyl.

7. The compound of claim 1 or 2, wherein ring A is selected from the group consisting of:

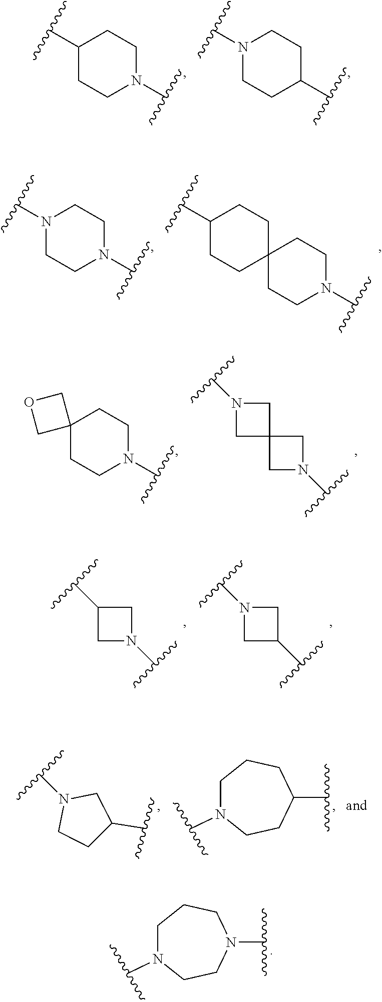

8. The compound of any one of claims 1 to 7, wherein R¹ is H, C₁₋₅alkyl, C₃₋₅alkenyl, C₃₋₆hydroxyalkyl or C₁₋₂alkylene-C₃₋₁₀cycloalkyl.

9. The compound of any one of claims 1 to 7, wherein R¹ is H, CH₃, CH═C(CH₃)₂, CH₂C(CH₃)₂OH, CH₂C(CH₃)₃, CH₂cylopropyl, or CH₂adamantyl.

10. The compound of any one of claims 1 to 7, wherein R¹ is H.

11. The compound of any one of claims 1 to 7, wherein R¹ is a C₁₋₆alkyl.

12. The compound of any one of claims 1 to 7, wherein R¹ is methyl, isopropyl, sec-butyl, or CH₂C(CH₃)₃.

13. The compound of any one of claims 1 to 7, wherein R¹ is C₁₋₆hydroxyalkyl.

14. The compound of claim 13, wherein R¹ is

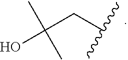 [SMILES: *CC(C)(C)O]

15. The compound of any one of claims 1 to 7, wherein R¹ is C₃₋₁₀cycloalkyl or C₁₋₃alkylene-C₃₋₁₀cycloalkyl.

16. The compound of claim 15, wherein the cycloalkyl group is cyclopropyl.

17. The compound of any one of claims 1 to 7, wherein R¹ is C₀₋₃alkylene-C₁₀cycloalkyl.

18. The compound of claim 1, wherein 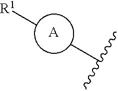
is selected from the group consisting of: 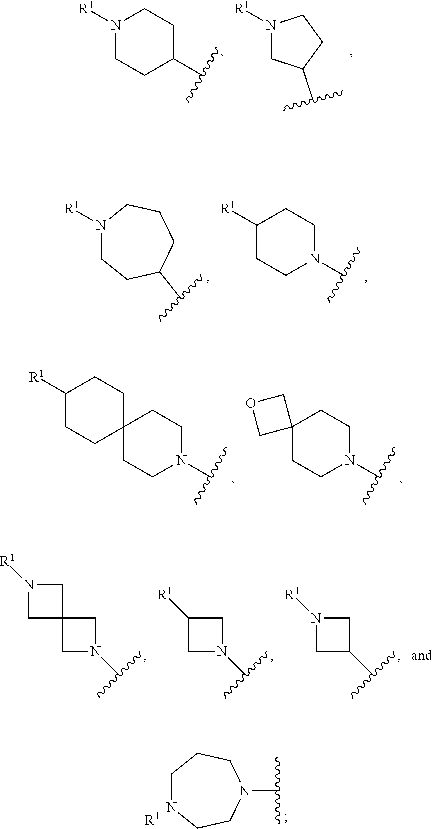
R¹ is selected from the group consisting of H, CH₃, 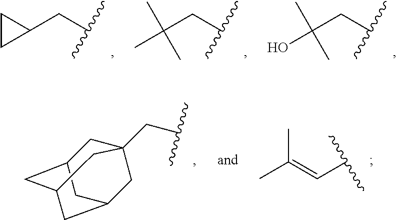
R² is H, F, Cl, or CH₃;
R³ is CH₃,
R⁴ is H or F; and
n is 0, 1, or 2.

19. The compound of claim 1, wherien 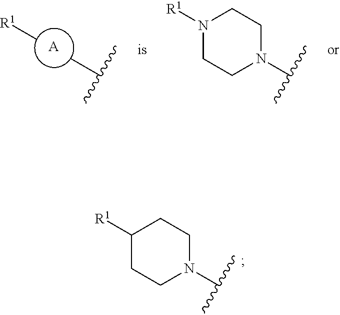
R¹ is selected from the group consisting of H, CH₃, 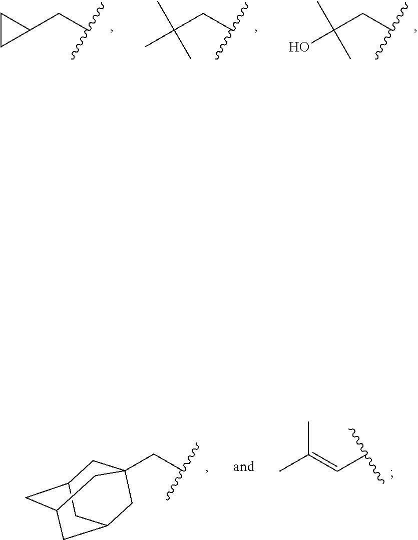
R² is H, F, Cl, or CH₃;
R³ is CH₃,
R⁴ is H or F; and
n is 0, 1, or 2.

20. The compound of claim 1, wherein 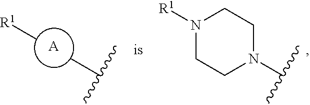
R¹ is selected from the group consisting of 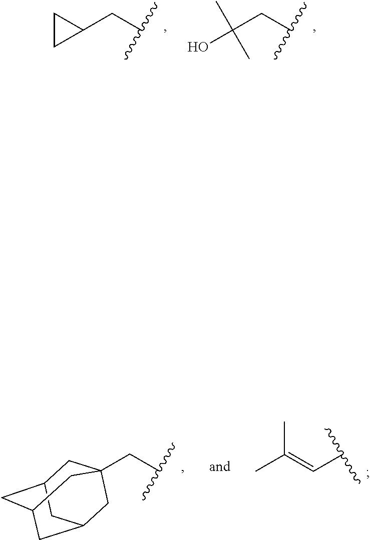
R² is H, F, Cl, or CH₃;
R³ is CH₃;
R⁴ is H or F; and
n is 0, 1, or 2.

21. The compound of any one of claims 1 to 20, wherein R² is H.

22. The compound of any one of claims 1 to 20, wherein R² is F.

23. The compound of any one of claims 1 to 20, wherein R² is CH₃.

24. The compound of any one of claims 1 to 23, wherein R³ is CH₃.

25. The compound of any one of claims 1 to 23, wherein n is 0.

26. The compound of any one of claims 1 to 24, wherein n is 1.

27. The compound of any one of claims 1 to 24, wherein n is 2.

28. The compound of claim 27, wherein each R³ is substituted at the same atom on ring A.

29. The compound of claim 27, wherein each R³ is substituted at different atoms on ring A.

30. The compound of any one of claims 1 to 29, wherein R⁴ is H or F.

31. The compound of any one of claims 1 to 29, wherein R⁴ is H.

32. A compound having a structure as recited in Table 1 or Table 2, or a pharmaceutically acceptable salt thereof.

33. The compound of any one of claims 1 to 32 in the form of a salt.

34. A pharmaceutical composition comprising the compound of any one of claims 1 to 33 and a pharmaceutically acceptable carrier.

35. A method of selectively inhibiting HDAC3 (in vitro or in vivo), the method comprising contacting a cell with an effective amount of the compound of any one of claims 1 to 33, or pharmaceutically acceptable salt thereof, or the composition of claim 34.

36. A method of selectively inhibiting HDAC1 or HDAC2 (in vitro or in vivo), the method comprising contacting a cell with an effective amount of the compound of any one of claims 1 to 33, or pharmaceutically acceptable salt thereof, or the composition of claim 34.

37. A method of selectively inhibiting HDAC1, HDAC2, and HDAC3 (in vitro or in vivo), the method comprising contacting a cell with an effective amount of the compound of any one of claims 1 to 33, or pharmaceutically acceptable salt thereof, or the composition of claim 34.

38. A method of treating a disease or disorder mediated by HDAC1 or HDAC2 in a subject in need thereof, the method comprising administering an effective amount of the compound of any one of claims 1 to 33, or pharmaceutically acceptable salt thereof, or the composition of claim 34, to the subject.

39. A method of treating a disease or disorder mediated by HDAC3 in a subject in need thereof, the method comprising administering an effective amount of the compound of any one of claims 1 to 33, or pharmaceutically acceptable salt thereof, or the composition of claim 34, to the subject.

40. A method of treating a disease or disorder mediated by HDAC1, HDAC2, and HDAC3 in a subject in need thereof, the method comprising administering an effective amount of the compound of any one of claims 1 to 33, or pharmaceutically acceptable salt thereof, or the composition of claim 34, to the subject.

41. A method of treating a neurological disorder such as Friedreich's ataxia, myotonic dystrophy, spinal muscular atrophy, fragile X syndrome, Huntington's disease, spinocerebellar ataxia, Kennedy's disease, amyotrophic lateral sclerosis, Niemann Pick, Pitt Hopkins, spinal and bulbar muscular atrophy, and Alzheimer's disease; an inflammatory disease; a memory impairment condition, frontotemporal dementia, or a drug addiction in a subject in need thereof, the method comprising administering to the subject an effective amount of the compound of any one of claims 1 to 33, or pharmaceutically acceptable salt thereof, or the composition of claim 34.

42. A method of treating Friedreich's ataxia, myotonic dystrophy, spinal muscular atrophy, fragile X syndrome, Huntington's disease, spinocerebellar ataxia, Kennedy's disease, amyotrophic lateral sclerosis, Niemann Pick, Pitt Hopkins, spinal and bulbar muscular atrophy, or Alzheimer's disease in a subject in need thereof, the method comprising administering to the subject an effective amount of the compound of any one of claims 1 to 33, or pharmaceutically acceptable salt thereof, or the composition of claim 34.

43. A method of treating Friedreich's ataxia in a subject in need thereof, the method comprising administering to the subject an effective amount of the compound of any one of claims 1 to 33, or pharmaceutically acceptable salt thereof, or the composition of claim 34.

44. A method of treating an infection in a subject in need thereof, the method comprising administering to the subject an effective amount of the compound of any one of claims 1 to 33, or pharmaceutically acceptable salt thereof, or the composition of claim 34.

45. A compound of any one of claims 1 to 33, or pharmaceutically acceptable salt thereof, or the composition of claim 34 for use as a medicament.

46. A compound of any one of claims 1 to 33, or pharmaceutically acceptable salt thereof, or the composition of claim 34 for use in the treatment of a neurological disorder such as Friedreich's ataxia, myotonic dystrophy, spinal muscular atrophy, fragile X syndrome, Huntington's disease, spinocerebellar ataxia, Kennedy's disease, amyotrophic lateral sclerosis, Niemann Pick, Pitt Hopkins, spinal and bulbar muscular atrophy, and Alzheimer's disease; an inflammatory disease; a memory impairment condition, frontotemporal dementia, or a drug addiction in a subject in need thereof.

47. A compound of any one of claims 1 to 33, or pharmaceutically acceptable salt thereof, or the composition of claim 34 for use in the treatment of Friedreich's ataxia, myotonic dystrophy, spinal muscular atrophy, fragile X syndrome, Huntington's disease, spinocerebellar ataxia, Kennedy's disease, amyotrophic lateral sclerosis, Niemann Pick, Pitt Hopkins, spinal and bulbar muscular atrophy, or Alzheimer's disease in a subject in need thereof.

48. A compound of any one of claims 1 to 33, or pharmaceutically acceptable salt thereof, or the composition of claim 34 for use in the treatment of Friedreich's ataxia in a subject in need thereof.


SEQUENCE LISTING (2 sequences)

SEQ ID NO: 1 - DNA, 20 bp, Artificial Sequence
  Synthetic Polynucleotide
cagaggaaacgctggactct

SEQ ID NO: 2 - DNA, 20 bp, Artificial Sequence
  Synthetic Polynucleotide
agccagatttgcttgtttgg